SEQUENCE LISTING (3 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1284 aa, Severe acute respiratory syndrome-related coronavirus
MFMPSSFSYSSWATCWLLCCLIILAKATMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 3 - protein, 1288 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

(19) United States
(12) Patent Application Publication
(10) Pub. No.: US 2024/0274239 A1
(43) Pub. Date: Aug. 15, 2024

(54) METHODS RELATED TO AN ALTERNATIVE CONFORMATION OF THE SARS-COV-2 SPIKE PROTEIN

(71) Applicants: CZ Biohub SF, LLC, San Francisco, CA (US); The Regents of the University of California, Oakland, CA (US)

(72) Inventors: Susan Marqusee, Oakland, CA (US); Shawn M. Costello, Oakland, CA (US); Sophie R. Shoemaker, Oakland, CA (US)

(73) Assignees: CZ Biohub SF, LLC, San Francisco, CA (US); The Regents of the University of California, Oakland, CA (US)

(21) Appl. No.: 18/569,742

(22) PCT Filed: Jul. 8, 2022
(86) PCT No.: PCT/US2022/036555

§ 371 Date: Dec. 13, 2023

Related U.S. Application Data
(60) Provisional application No. 63/287,278, filed on Dec. 8, 2021.
(60) Provisional application No. 63/220,388, filed on Jul. 9, 2021.

(51) Int. Cl. G16B 40/10; G01N 33/68; G16B 15/30

(52) U.S. Cl. CPC G16B 40/10; G01N 33/6845; G01N 33/6848; G16B 15/30; G01N 2333/165; G01N 2458/15

(57) ABSTRACT

Described are methods related to a newly discovered alternative conformation of the SARS COV 2 Spike protein.

### BACKGROUND

Spike protein from SARS-COV-2 (SARS-COV-2 Spike protein) is the primary target for current vaccines against COVID-19 and the focus of many therapeutic efforts (1-4). This large heavily glycosylated trimeric protein is responsible for cell entry via recognition of the host receptor angiotensin-converting enzyme 2 (ACE2) and membrane fusion (5-7). It is also the principal antigenic determinant of neutralizing antibodies (8). Shortly after release of the viral genome sequence, a version of SARS-COV-2 Spike protein ectodomain (termed “S-2P”) was designed to stabilize the pre-fusion conformation, and the structure was determined by cryo-electron microscopy (cryo-EM) (9, 10).

S-2P comprises the first ˜1200 amino acids of SARS-COV-2 Spike protein with two proline substitutions in the S2 domain designed to stabilize the pre-fusion conformation, mutations that abolish the furin-cleavage site, and the addition of a C-terminal trimerization motif (9). This version of SARS-COV-2 Spike protein, its structure, and others that followed, have been widely used for vaccine development and interpretation of many structure/function and epidemiological studies. To date there are more than 250 structures of SARS-COV-2 Spike protein ectodomains in the Protein Data Bank (11). The structural studies of SARS-COV-2 Spike protein, together with the functional studies, demonstrated that, similarly to other class 1 viral fusion proteins, SARS-COV-2 Spike protein is dynamic, sampling several different conformations during its functional lifecycle (12, 13).

The three individual receptor-binding domains (RBDs) of SARS-COV-2 Spike protein sample a so-called “up” state and a so-called “down” state. The up state exposes the ACE2-binding motif, and is therefore required for infectivity (7, 10, 14, 15). After receptor binding and cleavage between the S1 and S2 domains, SARS-COV-2 Spike protein undergoes a major refolding event that allows for membrane fusion, and adopts the stable post-fusion conformation (6, 7, 16-18).

Despite the wealth of structural information, there are very few experimental studies on the dynamics within the pre-fusion state of SARS-COV-2 Spike protein. The noted RBD up/down conformational transition has been monitored on the membrane via single molecule FRET and occurs on the order of seconds (19). Large computational resources have been devoted to molecular simulations of SARS-COV-2 Spike protein revealing a dynamic pre-fusion state with a range of accessible conformations, including the potential of a further opening of the RBD and N-terminal domain (NTD) away from the trimer interface (20, 21). Experimentally, the conformational landscape of SARS-COV-2 Spike protein has not been well interrogated, and the effects of perturbations, such as ligand binding (which include binding of both receptor and antibodies) or amino acid substitutions found in emerging variants of concern are unknown.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are, among others, the following non-limiting exemplary embodiments. Among the exemplary embodiments are methods of determining a distribution of a SARS CoV 2 Spike protein an aqueous solution between a first conformation and a second conformation, comprising the steps of: providing the aqueous solution of the SARS COV 2 Spike protein; performing hydrogen/deuterium exchange mass spectrometry (HDX-MS) analysis of the aqueous solution of the SARS COV 2 Spike protein, thereby generating HDX-MS analysis data; using a computer, calculating deuterium incorporation data from the HDX-MS analysis data; and, using a computer, determining, from the deuterium incorporation data, the distribution of the SARS COV 2 Spike protein in the aqueous solution between the first conformation and the second conformation. The SARS COV 2 Spike protein comprises one or more peptides having first deuterium incorporation data in the first conformation of the SARS COV 2 Spike protein and second deuterium incorporation data in the second conformation of the SARS COV 2 Spike protein.

Also included among the exemplary embodiments are methods of determining if a ligand is capable of stabilizing a first conformation and/or a second conformation of a SARS CoV 2 Spike protein, comprising the steps of: providing an aqueous solution comprising the SARS COV 2 Spike protein and the ligand; performing hydrogen/deuterium exchange mass spectrometry (HDX-MS) analysis of the aqueous solution, thereby generating HDX-MS analysis data; using a computer, calculating deuterium incorporation data from the HDX-MS analysis data; and, using a computer, determining, from the deuterium incorporation data, a distribution of the SARS COV 2 Spike protein in the aqueous solution between the first conformation and the second conformation wherein the ligand is capable of stabilizing the first conformation of the SARS COV 2 Spike protein when a proportion of the SARS COV 2 Spike protein found in the first conformation is increased in presence of the ligand as compared to absence of the ligand, and wherein the ligand is capable of stabilizing the second conformation of the SARS COV 2 Spike protein when a proportion of the SARS COV 2 Spike protein found in the second conformation is increased in the presence of the ligand as compared to the absence of the ligand. Also among the exemplary embodiments are methods of detecting binding of a ligand to a second conformation of a SARS-COV-2 Spike protein, comprising the steps of: providing an aqueous solution comprising the SARS-COV-2 Spike protein in the second conformation; contacting the ligand with the aqueous solution comprising the SARS-COV-2 Spike protein in the second conformation; and after the contacting, performing an in vitro analytical method to detect binding of the ligand to SARS-COV-2 Spike protein. Also among the embodiments of the present invention are methods of identifying a ligand capable of binding to a second conformation of a SARS-COV-2 Spike protein, comprising the steps of: providing an aqueous solution comprising the SARS-COV-2 Spike protein in the second conformation; contacting the ligand with the aqueous solution comprising the SARS-COV-2 Spike protein in the second conformation; and after the contacting, performing hydrogen/deuterium exchange mass spectrometry (HDX-MS) analysis to detect if one or more peptides of the SARS-COV-2 Spike protein that are more solvent exposed in the second conformation of the SARS-COV-2 Spike protein than in a first conformation of the SARS-COV-2 Spike protein become less solvent after the contacting with the ligand, thereby identifying the ligand as capable of binding to the second conformation of the SARS-COV-2 Spike protein.

Also included among the exemplary embodiments are methods of identifying a ligand capable of binding to SARS-COV-2 Spike protein, comprising the steps of: screening in silico a ligand library for candidate ligands capable of binding to a first conformation of the SARS-COV-2 Spike protein, a second conformation of the SARS-COV-2 Spike protein, or both to the first and the second conformation of the SARS-COV-2 Spike protein wherein three-dimensional models of the first conformation and the second conformation of the SARS-COV-2 Spike protein are computationally derived and incorporate solvent accessibility information based on deuterium incorporation data obtained by hydrogen/deuterium exchange mass spectrometry (HDX-MS) analysis; and, evaluating the candidate ligands identified in the screening steps through one or more in vitro analytical method for their ability to bind to the SARS-COV-2 Spike protein. Also included among the exemplary embodiments are methods of identifying a ligand capable of binding to a first conformation and/or a second conformation of a SARS-COV-2 Spike protein, comprising the steps of: identifying in silico a test ligand capable of interacting with the first conformation of the SARS-COV-2 Spike protein, the second conformation of the SARS-COV-2 Spike protein, or both to the first and the second conformation of the SARS-COV-2 Spike protein wherein three-dimensional models of the first conformation and the second conformation of the SARS-COV-2 Spike protein are computationally derived and incorporate solvent accessibility information based on deuterium incorporation obtained by hydrogen/deuterium exchange mass spectrometry (HDX-MS) analysis; and, evaluating the identified test ligand through one or more in vitro analytical method for its ability to bind to the SARS-COV-2 Spike protein.

These and other embodiments of the disclosure are described in detail below. For example, some other embodiments are directed to systems, devices, and computer readable media associated with methods described herein.

### BRIEF DESCRIPTION OF THE DRAWINGS

The present disclosure includes the following figures. The figures are intended to illustrate certain embodiments and/or features of the embodiments of the present invention, and to supplement any description(s) of the embodiments of the present invention. The figures do not limit the scope of the embodiments of the present invention, unless the written description expressly indicates that such is the case.

FIG. 1A is a schematic illustration of the pre-fusion-stabilized SARS-COV-2 Spike protein and a model of the trimeric pre-fusion conformation.

FIG. 1B is a schematic illustration of an Hydrogen-Deuterium Exchange Monitored by Mass Spectrometry (HDX-MS) experiment and the resulting mass distributions for a peptide that exists in either a one (left) or two (right) separable conformations. In order for the two conformations to result in this bimodal mass distribution, they must not interconvert during the timescale of the HDX experiment (hours). Rapid interconversion would result in a single mass distribution with the ensemble averaged mass profile.

FIG. 1C is a schematic representation of the deuterium uptake across the entire SARS-COV-2 Spike protein displayed on the full trimer (left) or a single protomer (right) after 1 minute of exchange.

FIG. 2A shows: on the left, a schematic illustration of SARS-COV-2 Spike monomer with all regions that have peptides with bimodal mass distributions indicated in darker shade; on the right, example mass spectra from two peptides (top: amino acid residues 982-1001, bottom: amino acid residues 878-903) with overlaid fitted Gaussian distributions that describe each protein conformation (the less exchanged A state and the more exchanged state B), as indicated.

FIG. 2B shows plots illustrating conformational preference for S-2P at 25° C., 4° ° C. and 37° C. At 25° C. S-2P converts from primarily state A to 50:50 A:B after ˜5 days. At 4° C. S-2P prefers state B, while at 37° C. S-2P prefers state A.

FIG. 2C shows line plots illustrating the kinetics of interconversion between the A and B conformation for different constructs of SARS-COV-2 Spike protein (S-2P, HexaPro, and UK S1 HexaPro, as indicated). Starting from an initial pre-fusion conformation (state A, 37° C.), samples were rapidly transferred to 4° C. and then assayed at 25° C. for conversion to state B over time. To estimate the fraction of conformation (state) A in each sample (“fraction state A,” plotted on the Y axis), peptides from two different regions (amino acid residues 982-1001 (circles) and amino acid residues 878-903 (triangles)) were fit to two Gaussians—for state A and state B, and the fraction of state A was calculated by taking the area under the fitted Gaussian for state A and dividing it by the sum of the areas under Gaussians for state A and state B. The data from both regions of each SARS-COV-2 Spike protein construct were used to determine the rate of interconversion.

FIG. 3A shows a diagram of the Spike structure with regions of interest highlighted.

FIG. 3B shows: on the left, a heat map showing the difference in peptide deuteration in the presence and absence of ACE2 (isolated RBD); in the middle, the deuterium uptake plots for three peptides of interest: amino acids 400-421 for the top plot, amino acids 453-470 for the middle plot, and amino acids 487-510 for the bottom; on the right, a schematic representation of the heat map on the structure of the RBD.ACE2 complex (PDB 6MOJ) (the structure of the RBD is shaded based on the maximum change shown in the heat map for that amino acid residue in any peptide).

FIG. 3C illustrates changes to two peptides of interest from HexaPro (amino acid residues 982-1001—region II peptide; amino acid residues 878-903—region III peptide) upon binding of ACE2 outside of the RBD. In the left two panels, the two peptides of interest are shown in the corresponding regions of spike structure in darker shade. In the schematic structure of region II, one N-terminal domain has been removed to visualize the peptide of interest. On the right are deuterium uptake plots for the peptides (top two plots—region II peptide; bottom two plots—region III peptide). The deuterium update plots for each peptide are show for state A (left two plots) and state B (right two plots). Since both peptides of interest are bimodal, deuterium uptake for each state can be quantified independently. When ACE2 bound to the canonical pre-fusion structure, State A, peptide 982-1001 (region II peptide) became more solvent-exposed and thus exchanged more. When ACE2 bound to state B, region II peptide did not become more exchanged (presumably because it was already maximally solvent-exposed). For peptide 878-903 (region III peptide), there was no effect on solvent exposure upon ACE2 binding in either state A or state B, indicating that region III peptide was not affected by ACE2 binding.

FIG. 3D shows the plots illustrating time course of interconversion in the presence of ACE2. Top plots are example mass spectra of S-2P peptide of amino acid residues 878-902 with and without ACE2, as labeled, before and after 24 hours of incubation at 25° C. The Gaussian for state A is shown in light grey; the Gaussian for State B is shown in dark grey.

The bottom plot is a dot plot of time (X-axis) versus fraction state A (Y-axis) for peptide of amino acid residues 878-902 in S-2P with ACE2 (“ACE2”) and without ACE2 (“apo”) over 24 hours. The plot illustrates that, after 24 hours, S-2P bound to ACE2 preferred state B.

FIG. 4A shows, on the right, example mass spectra for two bimodal HexaPro peptides with (“3A3 HexaPro”) and without (“Apo HexaPro”) 3A3 antibody (top two plots—peptide of amino acid residues 982-1001; bottom two plots—peptide of amino acid residues 878-903). The peptide of amino acid residues 878-903 showed no change in the presence of 3A3 antibody, which indicated that the amount of state A and state B did not significantly change at the time the HDX-MS data were taken (13 minutes after adding 3A3 antibody). The peptide of amino acid residues 982-1001 showed significant protection in the presence of 3A3 antibody, shifting the distribution belonging to state B to a deuteration amount indistinguishable from state A. On the left is a schematic representation of HexaPro structure indicating the location of the two bimodal peptides in a darker shade.

FIG. 4B illustrates the kinetics of interconversion of S-2P in the presence of 3A3 antibody. The addition of 3A3 antibody accelerated the rate of conversion to state B at 4° C. The binding of 3A3 antibody prevented the return to state A at 37° C.

FIG. 5 shows a schematic of the energy landscape for the SARS-COV-2 Spike ectodomain. Three different conformational states are schematically depicted: the canonical pre-fusion (“Pre-fusion Ensemble”), the expanded open trimer (“Expanded Open Trimer”), and the post-fusion conformation (“Postfusion”). The pre-fusion conformation contains all four RBD states (0, 1, 2, or 3 up). The relative energies and barrier heights and the placement of the open trimer along the reaction coordinate are shown for illustration only.

FIG. 6 shows peptide coverage maps illustrating peptide coverage and redundancy at each amino acid residue for all HDX-MS experiments.

FIG. 7 shows a plot illustrating exemplary results of back-exchange control experiments. The plot is a cumulative histogram of the fractional deuterium maintained during workup of a fully deuterated sample. Fraction Max exchange plotted on the X-axis is corrected for the 90% D20 experimental conditions. Plotted on the Y-axis is the number of peptides that have a given level of back exchange (plotted on the X-axis) or less.

FIG. 8A schematically illustrates HDX-MS results as a function of time, showing deuterium uptake for each S-2P experimental time point mapped to the structure of the pre-fusion trimer and a single protomer (model from (24)). Per residue deuteration was calculated from all peptide data by HDExaminer 3.

FIG. 8B schematically illustrates HDX-MS results as a function of time, showing deuterium uptake for each Apo-RBD experimental time point mapped to the structure of the RBD (single RBD from a full-length spike trimer model from (24)).

FIG. 9A shows S2P bimodal peptide spectra observed in continuous labeling HDX-MS experiments for all peptides with observed bimodal behavior for S-2P.

FIG. 9B shows S2P bimodal peptide spectra observed in continuous labeling HDX-MS experiments for all peptides with observed bimodal behavior for HexaPro.

FIG. 9C shows bimodal peptide spectra obsereved in pulse-labeling HDX-MS experiments for the bimodal peptides used to quantify the relative populations of state A and B. The spectra are shown with the resulting Gaussian fits overlaid.

FIG. 10 schematically illustrates comparison of HDX conducted of isolated RBD of SARS-COV-2 Spike protein and RBD in S-2P. The left panel is a heat map showing the difference in peptide deuteration for isolated RBD compared to the RBD in S-2P. The middle panels show exemplary uptake plots of isolated RBD and S-2P RBD. The right panel shows a structure of the RBD (model of a single RBD taken from a full-length spike trimer model from (24)) rendered based on the maximum change shown in the heat map for that amino acid residue in any peptide. For reference, spheres are shown denoting the beginning and end of the peptides displayed in the uptake plots.

FIG. 11A shows, in the top panel, Superose 6 increase 3.2/300 (SEC) traces from S-2P after incubation at 37° C. and 4° C. FIG. 11A shows, in the bottom panel, MS spectra of a bimodal peptide (amino acid residues 878-902) from each sample taken immediately before SEC experiment.

FIG. 11B shows, in the top panel, schematic structure of the T4 Fibritin trimerization domain (PDB 1RFO) with the peptide followed by HDX-MS indicated in darker shade, and, in the bottom panel, peptide deuterium uptake at one minute as a function of fraction state B.

### TERMS AND CONCEPTS

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

As used in the present disclosure, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.”

When a numerical range is provided in the present disclosure, the numerical range includes the range endpoints unless otherwise indicated. Unless otherwise indicated, numerical ranges include all values and subranges tin the present disclosure, as if explicitly written out.

The terms “about” and “approximately,” as used in the present disclosure, shall

generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” or “approximately X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.01X. In another example, the terms “about” or “approximately” in relation to a reference numerical value can include a range of values plus or minus 10%, 9%, 8%, 7%, 6%, 5%, 4%, 3%, 2%, or 1% from that value. Thus, expressions “about X” or “approximately X” are intended to describe a claim limitation of, for example, “0.98X.” Numerical quantities given in the present disclosure are approximate unless stated otherwise, meaning that the term “about” or “approximately” can be inferred when not expressly stated. When the terms “about” or “approximately” are applied to the beginning of a numerical range, they apply to both ends of the range. Where a series of values is prefaced with the terms “about” or “approximately,” these terms are intended to modify each value included in the series.

“Virus” and the related terms and expressions are used in both the plural and singular senses. “Virion” refers to a single virus. For example, the expression “coronavirus virion” refers to a coronavirus particle.

The terms “peptide,” “polypeptide” or “protein” are used to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology.

The terms “oligonucleotide,” “polynucleotide” or “nucleic acid” encompass DNA or RNA molecules, including the molecules produced synthetically or by recombinant technology. Oligonucleotides, polynucleotides or nucleic acids may be single-stranded or double-stranded.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile), leucine (Leu), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). In the broadest sense, the naturally occurring amino acids can be divided into groups based upon the chemical characteristic of the side chain of the respective amino acids.

By “hydrophobic” amino acid is meant either His, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His. This grouping of amino acids can be further sub-classed as follows: by “uncharged hydrophilic” amino acid is meant either Ser, Thr, Asn or Gln. By “acidic” amino acid is meant either Glu or Asp. By “basic” amino acid is meant either Lys, Arg or His.

The term “variant,” when used in the present disclosure in reference to a protein or a polypeptide, encompasses homologues, variants, isoforms, fragments, mutants, modified forms and other variations of the protein, polypeptide or amino acid sequences described in this document. The term “homologous,” “homologues” and other related terms used in this document in reference to various amino acid, are intended to describe a degree of sequence similarity among amino acid sequences, calculated according to an accepted procedure. Homologous sequences may be at least 60%, 65%, 70%, 75%, 80%, 85%, 90%, 91%, 92%, 93%, 94%, 95%, 96%, 97%, 98% 99% or 100% homologous (or also described as having at least 60%, 65%, 70%, 75%, 80%, 85%, 90%, 91%, 92%, 93%, 94%, 95%, 96%, 97%, 98% 99% or 100% “sequence identity” or “sequence similarity.” As used herein, “percent homology” (or “sequence identity,” or “sequence similarity”) of two amino acid sequences is determined using the algorithm of Karlin and Altschul, which is incorporated into the NBLAST and XBLAST programs, available for public use through the website of the National Institutes of Health (U.S.A.). To obtain gapped alignments for comparison purposes, Gapped BLAST is utilized. When utilizing BLAST and Gapped BLAST programs, the default parameters of the respective programs (e.g., XBLAST and NBLAST) are used. “Percent homology” may be used in this document to describe fragments, variants or isoforms of amino acids sequences, but other ways of describing fragments, variants or isoforms may be employed alternatively to or in conjunction with homology.

The expression “conservatively modified variant” and related expression may apply to amino acid sequences, as well to nucleic acid sequences encoding amino acid sequence. Substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

A “domain” of a protein or a polypeptide refers to a region of the protein or polypeptide defined by structural and/or functional properties. Exemplary function properties include enzymatic activity and/or the ability to bind to or be bound by another protein or non-protein entity. For example, coronavirus Spike protein contains S1 and S2 domains.

The term “binding site” and related terms and expression refer to an area on the protein wherein a ligand can interact with such as a region, which can be located on the surface or interior of the protein molecule. Binding site can have a concave surface presenting amino acid residues in a suitable configuration for binding ligands, such as, but not limited to, low molecular weight compounds (which can be referred to as “small molecules”). The mobility of a protein molecule can permit opening, closing, and adaptation of binding site to regulate binding processes. The influence of protein flexibility on binding sites can vary from small changes to an already existent sites to the formation of a completely new binding site.

The term “oligomer” and related terms, when used in reference to polypeptides or proteins, refer to complexes formed by two or more polypeptide or protein monomers, which can also be referred to as “subunits” or “chains.” For example, a trimer is an oligomer formed by three polypeptide subunits.

The term “conformation,” “conformer” and related terms and expressions, when used in reference to polypeptides or proteins, refer to a distinct three-dimensional arrangement of the atoms that make up a protein, or a set of three-dimensional arrangements of atoms that make up a protein that is kinetically distinct from another set.

The term “ligand” and the related terms are used in the present disclosure refer to a compound or compounds that form a complex with SARS-COV-2 Spike protein. The term “ligand” encompasses all compounds, regardless of their size or origin. For example, inorganic molecules, organic molecules, small molecules, biological molecules, non-biological molecules are all encompassed by the term “ligand.”

The term “antibody” and the related terms, in the broadest sense, are used in the present disclosure to denote any product, composition or molecule that contains at least one epitope binding site, meaning a molecule capable of specifically binding an “epitope” - a region or structure within an antigen. The term “antibody” encompasses whole immunoglobulin (i.e., an intact antibody) of any class, including natural, nature-based, modified, and non-natural (engineered) antibodies, as well as their fragments. The term “antibody” encompasses “polyclonal antibodies,” which react against the same antigen, but may bind to different epitopes within the antigen, as well as “monoclonal antibodies” (“mAbs”), meaning a substantially homogenous population of antibodies or an antibody obtained from a substantially homogeneous population of antibodies. The antigen binding sites of the individual antibodies comprising the population of mAbs are comprised of polypeptide regions similar (although not necessarily identical) in sequence. The term “antibody” also encompasses fragments, variants, modified and engineered antibodies, such as those artificially produced (“engineered), for example, by recombinant techniques. For instance, the term “antibody” encompasses, but is not limited to, chimeric antibodies and hybrid antibodies, antibodies with dual or multiple antigen or epitope specificities, and fragments, such as F(ab')2, Fab′, Fab, hybrid fragment, single chain variable fragments (scFv), “third generation” (3G) fragments, fusion proteins, single domain and “miniaturized” antibody molecules, and “nanobodies.”

As used herein, the terms “small molecule,” “small organic molecule” and “small inorganic molecule” includes molecules (either organic, organometallic, or inorganic), organic molecules, and inorganic molecules, respectively, which have a molecular weight of more than about 50 Da and less than about 2500 Da. Small organic (for example) molecules may be less than about 2000 Da, between about 100 Da to about 1000 Da, or between about 100 Da to about 600 Da, or between about 200 Da to about 500 Da.

The term “interaction” and the related terms refer to a type of physical or chemical interaction of one or more molecular subsets with itself (intramolecular) or other molecular subsets (intermolecular) or with components of an environment (environmental). Interaction types may be either enthalpic or entropic in nature and may reflect either nonbonded or bonded interactions. The forces that mediate the interactions between atoms and molecules may be referred to as “binding forces.” Examples of nonbonded interaction types include, but are not limited to, electrostatic interactions, van der Waals (or dispersion) interactions between time-varying dipole moments (often related to steric complementarity), short range repulsion between overlapping atomic orbitals, hydrogen bonding, interactions involved with metal ion coordination, or interactions with one or more ordered or structural waters. Other examples of nonbonded interaction types may also include one or more solvation effects such as electrostatic desolvation (including self-reaction field polarization effects, solvent screening in a dielectric medium or interactions with a solvent-based ionic atmosphere), the hydrophobic effect, cavitation energy, and surface tension. Examples of bonded interactions include, but are not limited to, the intramolecular strain associated with distortions of equilibrium bond lengths, angles, torsions, etc., or the energy gap between cis-trans modes or the energy differential associated with changes in chirality of one or more chiral center. Examples of entropic-based interactions include the loss of conformational entropy of molecular subsets (including loss of rotameric entropy for protein side chains) upon binding or the favorable entropy gain obtained by the release of one or more ordered waters. Other more exotic interaction types may include x-x stacking, charge transfer, or other quantum mechanical phenomena.

The term “hydrogen-bonding,” “hydrogen bonds,” and related terms relate to a partially electrostatic attraction between a hydrogen (H) which is bound to a more electronegative atom such as nitrogen (N) or oxygen (O) and another adjacent atom bearing a lone pair of electrons. For example, when it is stated that the nitrogen acts as a “hydrogen bond donor” it means that a hydrogen (H) bound to a nitrogen (N) is donated by the nitrogen as it electrostatically attracted to or accepted by an adjacent atom bearing a lone pair of electrons such as an oxygen. Similarly, when it is stated that an oxygen acts as a “hydrogen bond acceptor,” it means that a hydrogen (H) bound to a more electronegative atom such as nitrogen (N) is electrostatically attracted to or “accepted by” an adjacent atom such as oxygen bearing a lone pair of electrons. Sometimes the hydrogen bonded atoms are called out without explicitly stating the origin and presence of an intermediate hydrogen atom. The term “hydrogen bonding” is used wherever LigPlot Plus software predicts a hydrogen bonding interaction using its algorithm and applied parameters of 3.35 Å for maximum distance between hydrogen bond donor and acceptor. Not all hydrogen bonds may actually be in place simultaneously; this is evident for atoms that are shown to form 4 putative hydrogen bonds, where however, at any given time only 3 hydrogen bonds are chemically possible. In general, although crystal structures such as the co-crystal structural information herein does not directly show or detect hydrogen bonding, the software used to describe the co-crystal does predict such H-bonding exists. Therefore, throughout the disclosure when a H-bond is present and described, it may be said to be “predicted” by software to be present.

The term “ionic bonding” and related terms, such as “ionic interaction,” include a type of chemical bond that involves the electrostatic attraction between oppositely charged ions, and is the primary interaction occurring in ionic compounds.

The term “van der Waals interaction” and related terms include weak, short-range electrostatic attractive forces between uncharged molecules, arising from the interaction of permanent or transient electric dipole moments.

The term “π-π interaction or π-π stacking” and related terms include attractive, noncovalent interactions between aromatic rings that are oriented either roughly parallel or roughly perpendicular (such as in “edge-face” interactions) to each other, since they contain x bonds.

The term “steric interactions,” “steric effects” and the related terms describe molecular and/or atomic interactions that may arise in a number of ways. For example, steric effects may result from repulsions between valence electrons or nonbonded atoms, leading to in an increase in the energy of the system. In the formation of a ligand-receptor complex, any group of atoms that is in van der Waals contact with the receptor or the biomolecule can be or is involved in the binding event. If a ligand binding pocket can adjust to any ligand, then no steric effect will be observed. If, however, the binding pocket has limited conformational flexibility, and this flexibility is not equivalent in all directions, then a steric effect will be observed. The steric effect will be dependent on conformational states, and the minimal steric interaction principle will probably be observed. This principle states that a substituent whose steric effect is conformationally variable will prefer a conformation that minimizes steric repulsions and will give rise to the smallest steric strain.

The term “affinity formulation” and the related term refer to the energy model used to calculate approximate quantitative values for a given interaction type for a configuration associated with a molecular combination. Typically, there may be many different affinity formulations for a given interaction type from which to choose. The choice of affinity formulation may affect the amount of error associated with the quantitative approximation of a given interaction type. The choice of affinity formulation may also involve very different levels of modeling sophistication and hence computational complexity. A given affinity formulation may require one or more molecular descriptors for evaluation. Two different affinity formulations for a given interaction type may require a very different set of molecular descriptors, while others may share multiple molecular descriptors in common. For example, electrostatic interactions may be modeled according to an affinity formulation involving the use of a modified form of Coulomb's law with distance-dependent dielectric function as applied to a set of partial charges assigned to atomic centers in each molecular subset via use of a suitable force field. In another example, both electrostatic and electrostatic desolvation interactions may be modeled according to an affinity formulation involving a solution of the Poisson-Boltzmann equation (linear or nonlinear) along with an assumption of point charges embedded in solute spherical cavities with size defined by van der Waal radius of each atom and the solute spheres placed in a homogeneous dielectric medium representing water with and possibly containing an ionic atmosphere. Alternatively, electrostatic interactions may be modeled based on quantum-mechanical solution of electronic ground states for each molecular subset. In most scenarios the modified Coulomb with distance-dependent dielectric formulation will be cheaper to compute but less accurate than a Poisson-Boltzmann-based formulation let alone a full quantum-mechanical solution. As further examples, van der Waals interactions may be modeled according to an affinity formulation based on use of a generalized Lennard-Jones potential or alternatively based on a steric complementarity. Hydrogen-bonding interactions may be modeled according to an affinity formulation based on use of a 12-10 Lennard-Jones potential with an angular weighting function or by rescaling of partial charges and van der Waals radii of hydrogen bond donor and acceptor atoms such as that found in the Amber force field. The hydrophobic effect may be modeled according to an affinity formulation based on the fragmental volume approach or the solvent accessible surface area-based formalism. Intramolecular strain associated with dihedral changes may be modeled according to an affinity formulation based on use of Pitzer potentials or by inverse Gaussian torsional constraints. As yet another example, instead of using a Poisson Boltzmann-based formulation, electrostatic desolvation for a configuration may be modeled via an affinity formulation based on use of a variant of the Generalized Born approximation.

The term “computation strategy” herein refers to the computational technique used to quantitatively evaluate a given affinity formulation for one or more interaction types. The choice of computation strategy may be influenced by the available computational systems, apparatus, means and/or methods, the available memory capacity, and/or computing time constraints. As an example of different computational strategies for the same affinity formulation, consider the electrostatic interaction for target-ligand combination, for which a modified Coulombic affinity formulation with distance-dependent dielectric may be computed according to a computation strategy involving direct summation of pair-wise calculation between all possible pairs of partial charges across the protein and ligand. For a ligand with 100 atoms and a protein with 3000 atoms, this would entail the calculation of 300 K intermolecular distances let alone the number of distinct intramolecular pairs. An alternative computation strategy is to instead utilize a probe grid map approximation, whereby an electrostatic potential function associated with source charges on the protein is evaluated and stored on 3-D grid for coordinate locations enclosing the protein. Then for each ligand charge a corresponding electrostatic potential value is accessed from memory (or other storage) and the direct product of the charge and the potential is then accumulated over all charges in the ligand. This may significantly reduce computational effort especially in the context of screening a molecule library where many molecular combinations may feature the same target protein but different ligands. Of course, the probe grid map approximation may require significant storage in order to reduce numerical errors related to variation of the potential function. Moreover, such an approximation is only suitable when the source charges of the protein do not change positions between different configurations. An alternative for a target protein featuring a flexible binding pocket, may be to use a hybrid computation strategy involving the use of the pair-wise strategy for the portion of the protein containing mobile source charges and the probe grid map strategy for the remainder of the protein. In general, various different computation strategies may be applied to other affinity formulations for other interaction types. On the other hand, the choice of computation strategy may be limited by the nature of the affinity formulation or interaction type in question. For example, it is unlikely that one would a strategy appropriate for evaluation of intermolecular electrostatics interactions to instead compute intramolecular strain components involving bonded interactions. Other types of computational strategies exist than those based on pair-wise (e.g.,. interactions between pairs of atoms) or map or potential field (e.g., interactions of an atom with a potential field) calculations. For example, the evaluation of a Generalized Born solvation model based on the calculation of either volume integrals over the solvent excluded volume or on the calculation of surface integrals on the solvent accessible surface area. As yet another example, various formulations of bonded interactions may be evaluated according to a computation strategy featuring traversal of an appropriate data structure containing relevant coordinate and bond descriptors.

An “affinity function” is a composition of affinity components each of which corresponds to a combination of an interaction type, an affinity formulation, and a computation strategy. An affinity component may represent interactions for the whole or parts of one or more molecular subsets. An affinity function may contain multiple affinity components relating to the same interaction type. For example, two affinity components may represent the same interaction type but differ in either their affinity formulation and/or their computation strategy. Each distinct molecular configuration for a given molecular combination may produce different quantitative results for an affinity component and hence for the corresponding affinity function. In one embodiment, the analysis of a molecular combination may be based on determination of the configuration with the best value for the affinity function. In other embodiments, multiple favorable values for the affinity function corresponding to molecular configurations associated with one or more potential binding modes may be considered. In yet another embodiment, multiple affinity functions may be computed on one or more configurations of a molecular combination and some decision or action based on their joint consideration, such as for example the scenario of consensus scoring of a small finite number of configurations for each molecular combination explored in the course of screening a molecule library against a target molecule.

### DETAILED DESCRIPTION

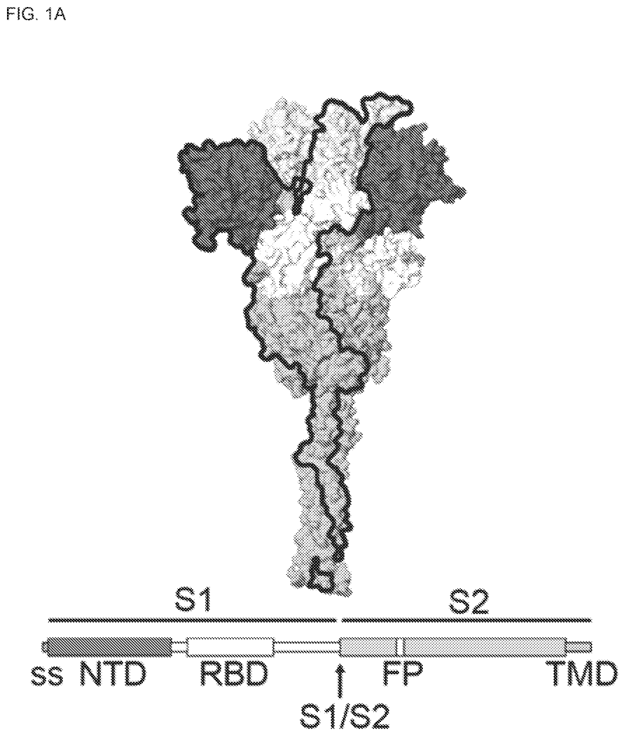

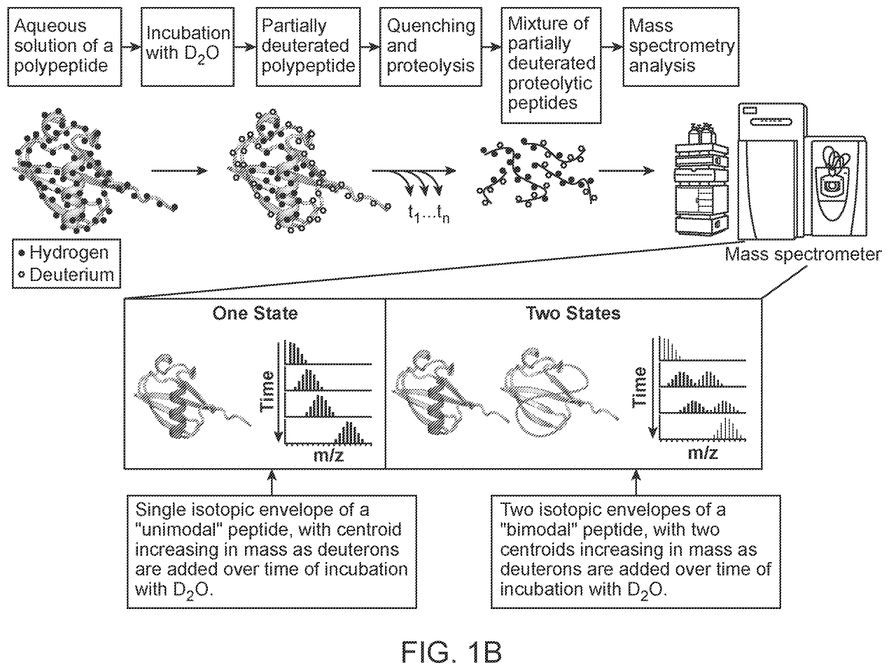

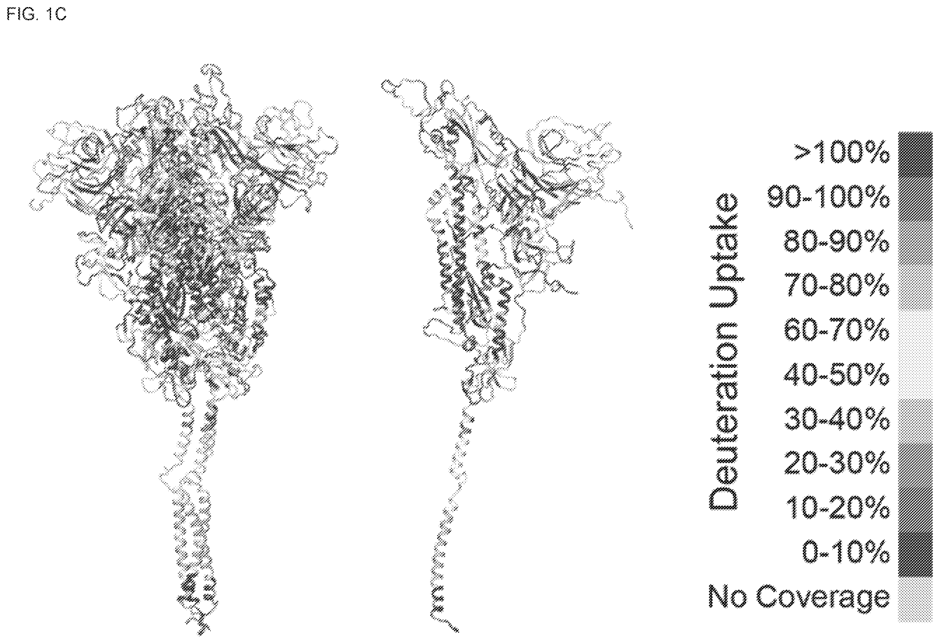

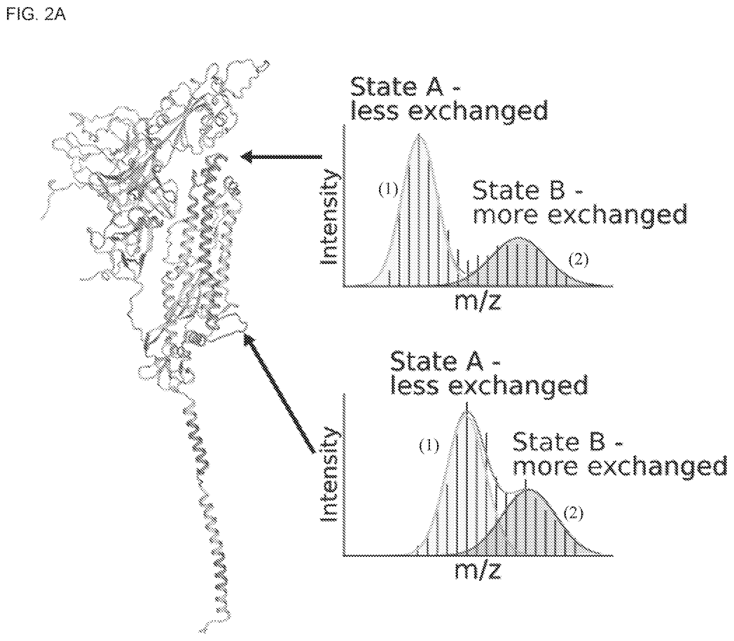

Described in the present disclosure are methods related to a newly discovered alternative conformation of SARS-COV-2 Spike protein. The inventors discovered, and the present disclosure describes, that SARS-COV-2 Spike ectodomain reversibly samples an alternative conformation, in addition to the previously known canonical, resolved by cryo-EM, pre-fusion conformation (which may also be referred to in the present disclosure as “state A,” “conformation A,” “conformer A,” “first state,” “first conformation,” or by other related terms or expressions). The inventors used hydrogen deuterium exchange paired with mass spectrometry (HDX-MS) to probe the energy landscape of the soluble pre-fusion ectodomain of SARS-COV-2 Spike protein, as well as the effects of ligand binding and sequence variation on the conformational landscape of SARS-COV-2 Spike protein. HDX-MS offers an ideal complement to the ever-growing number of structural studies on the SARS-COV-2 Spike protein, providing information on its conformational ensemble and dynamics. HDX-MS monitors the time course of exchange of amide hydrogens on the peptide backbone with the hydrogens in the solvent (see the description further in this document and FIG. 1B). An individual amide's ability to undergo exchange is directly related to its structure and stability (22, 23).

Using HDX-MS, the inventors found that, in addition to the conformation known from the pre-fusion structure determined by cryo-EM, SARS-COV-2 Spike protein adopts an alternative conformation that interconverts slowly with the canonical pre-fusion conformation (“state A”). This new conformation (which may also be referred to in the present disclosure as “state B,” “conformation B,” “second state,” “second conformation,” “alternative conformation,” or by other related terms or expressions) contains easily accessible receptor-binding domains (RBDs) and a large and unique solvent accessible surface area that is buried in the canonical pre-fusion conformation. For example, conformation B contains an exposed conserved trimer interface, which is buried in the canonical pre-fusion conformation of SARS-COV-2 Spike protein. Based on this finding, the inventors described conformation B of SARS-COV-2 Spike protein, which is trimeric, as “an open trimer.” The inventors realized that conformation B exposes potential surfaces of SARS-COV-2 Spike protein that may be important for antibody and ligand recognition. As further described in the present disclosure, population of state B SARS-COV-2 Spike protein and kinetics of interconversion between states A and B are modulated by receptor binding, antibody binding, and sequence variants observed in the natural population. Knowledge concerning various aspects of B state of SARS-COV-2 Spike protein is useful for improving SARS-COV-2 diagnostics, therapeutics, and vaccines.

An increase in the formation of state B upon binding of ACE2 by SARS-COV-2 Spike protein observed by the inventors suggested to them that state B may be a functional intermediate. For example, state B may be an intermediate along the pathway to S1 shedding during the transition of SARS-COV-2 Spike protein from the irreversible transition from pre-fusion conformation to the post-fusion conformation. This irreversible transition is not possible in the soluble ectodomain version of SARS-COV-2 Spike protein, which does not contain the proteolytic cleavage site. If state B is an “on-pathway” intermediate, ligands, including, but not limited to, antibodies, that trap (stabilize) SARS-COV-2 Spike protein in state B may block the protein along the pathway to fusion. Also, the ligands that act on the transition state and increase the rate of formation of state B may promote premature formation of the post-fusion conformation of SARS-COV-2 Spike protein, and thus aid in its neutralization during SARS-COV-2 infection. Alternatively, if formation of state B is “off-pathway,” ligands that favor state B may essentially trap SARS-COV-2 Spike protein in an inactive conformation (38), again, aid in its neutralization during SARS-COV-2 infection. The inventors conceived, that, in either situation, state B of SARS-COV-2 Spike protein is an important target for therapeutic applications.

The newly discovered conformation of SARS-COV-2 Spike protein presents new druggable sites. Since state B of SARS-COV-2 Spike protein contains solvent accessible surface area that is buried in the canonical pre-fusion conformation, state B exposes new binding sites for recognition by ligands, such as, but not limited to, polypeptides, antibodies or fragments thereof, and small molecules. Some of the newly discovered solvent accessible regions of SARS-COV-2 Spike protein are located in its most highly conserved part, the S2 trimer interface. Accordingly, ligands binding to the newly discovered binding sites of state B may be broadly efficacious against a range of coronaviruses, including, but not limited to, variants of concern of SARS-COV-2. The inventors discovered that antibody 3A3 represents one such potential ligand. The newly discovered solvent accessible regions in state B of SARS-COV-2 Spike protein present an attractive target for neutralizing antibodies that would provide protection across a range coronaviruses. Accordingly, amino acid sequences of the newly discovered solvent accessible regions of SARS-COV-2 Spike protein may be useful as antigens to be incorporated into anti-coronavirus vaccines.

Detection of newly discovered state B is useful in a variety of contexts, one of which is measurement of ligand binding affinities of SARS-COV-2 Spike protein in both research and diagnostic applications. The inventors discovered that state B is ubiquitous among in vitro preparations of the SARS-COV-2 Spike protein. The inventors found evidence of state B conformation in samples of every variant of SARS-COV-2 examined, excluding the disulfide-locked variant discussed further in these disclosures. Many biochemical and diagnostic assays use isolated SARS-COV-2 Spike protein, and many laboratories store its solutions at 4° C., the conditions under which state B is favored. Given that states A and B are expected to have differing affinities for at least some ligands, the temperature and time-dependent changes in the distribution of SARS-COV-2 Spike protein molecules between state A and state B in any given sample affects quantitative analysis of binding affinities. Accordingly, evaluating the conformational state of SARS-COV-2 Spike protein in solution with respect to states A and B is important for accurate measurements of ligand binding affinities of SARS-COV-2 Spike protein, which is, in turn, important for improving the accuracy of both research and diagnostic assays that measure binding of ligands to SARS-COV-2 Spike protein.

In sum, the inventors have found that SARS-COV-2 Spike protein ectodomain reversibly samples a newly discovered open-trimer conformation (state B). State B is similar in energy to the well-characterized canonical pre-fusion conformation determined by cryo-EM (state A), but has a different structure that exposes a highly conserved region of SARS-COV-2 Spike protein. The fraction of SARS-COV-2 Spike protein found in each of the two conformations in solution depends on various factors, including, but not limited, temperature, ligands, and amino sequence of SARS-COV-2 Spike protein. The inventors observed that mutations in SARS-COV-2 Spike protein sequence, ACE2 binding, and antibodies all affect the kinetics and energetics of the conformational state of SARS-COV-2 Spike protein. Thus, quantitative measurements characterizing that involve SARS-COV-2 Spike protein, such as, but not limited to, in vitro binding assays of SARS-COV-2 Spike protein ligands, need to be evaluated for possible effects of SARS-COV-2 Spike protein conformation state. The inventors also found that an antibody specific for state B of SARS-COV-2 Spike protein and can bind and neutralize SARS-COV-2 in in vitro binds specifically to state B, which identifies state B and SARS-COV-2 Spike protein peptides exposed in state B as important targets for both therapeutics and vaccine development.

Based on their discoveries described in the present disclosure the inventors conceived various methods utilizing the newly discovered alternative confrontation of the SARS-COV-2 Spike protein. For example, the present disclosure describes, among other things, methods of determining a distribution of the SARS-COV-2 Spike protein an aqueous solution between a first conformation and a second conformation, methods of determining if a ligand is capable of stabilizing a first or a second conformation of a SARS-COV-2 Spike protein, methods of detecting binding of a ligand to a first conformation or a second conformation of a SARS-COV-2 Spike protein, methods of identifying a ligand capable of binding to a first conformation or a second conformation of a SARS-COV-2 Spike protein.

The above and other methods described in the present disclosure may utilize a SARS-COV-2 Spike protein (or its fragment) found in the second conformation (state B), for example, in an aqueous solution, although other types of preparations, such are frozen or crystallized preparations are also envisioned. Some embodiments of the methods described in the present disclosure may utilize a SARS-COV-2 Spike protein (or its fragment) found predominantly in the second conformation (state B), meaning that >50%, at least 60%, at least 70%, at least 75%, at least 80%, at least 85%, at least 90%, at least 95%, or at least 99% of a SARS-COV-2 Spike protein in a particular preparation is in the second conformation. Some embodiments of the methods described in the present disclosure may utilize a computation model of SARS-COV-2 Spike protein or its fragment in the second conformation (state B).

The inventors also discovered that certain conditions, such as exposure to temperatures from above freezing and up to approximately 25° C., or binding to certain ligagnts, stimulated conversion of a SARS-COV-2 Spike protein from the first conformation to the second conformation. The inventors envisioned that various methods, such as, but not limited to, exposure to one or more ligands, various temperatures, pressures, pH, ionic strengths, surfactants, amino acid mutations (for example, substitutions), posttranslational modifications, etc., may be used to effect or stimulate such conversion. Accordingly, the inventors envisioned methods of stabilizing and/or producing the second conformation (state B) of a SARS-COV-2 Spike protein. Such methods are included among the embodiments of the present invention.

The present disclosure also described methods that involve computational (in silico) screening of a ligand library for candidate ligands capable of binding to a second conformation of the SARS-COV-2 Spike protein, as well as methods of computationally (in silico) identifying a test ligand capable of interacting with a second conformation of the SARS-COV-2 Spike protein. Such methods utilize the three-dimensional model of the second conformation of the SARS-COV-2 Spike protein that is computationally derived and incorporates solvent accessibility information based on deuterium incorporation data obtained by hydrogen/deuterium exchange mass spectrometry (HDX-MS) analysis.

The methods described in the present disclosure may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

### I. CORONAVIRUS SPIKE PROTEIN

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. Some examples of human coronaviruses are: Human coronavirus 229E (HCoV-229E); Human coronavirus OC43 (HCoV-OC43); Severe acute respiratory syndrome coronavirus (SARS-COV); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-COV), also known as novel coronavirus 2012 and HCoV-EMC; and Severe acute respiratory syndrome coronavirus 2 (SARS-COV-2), also known as 2019-nCOV or “novel coronavirus 2019.” In human, SARS-COV-2 causes coronavirus disease termed COVID-19, which can cause severe symptoms and death.

Spike protein (which can also be referred to as “Spike” or “S protein”) is a coronavirus surface protein that is able to mediate receptor binding and membrane fusion between a coronavirus virion and its host cell. Characteristic spikes on the surface of coronavirus virions are formed by ectodomains of homotrimers of Spike protein. Coronavirus Spike protein is highly glycosylated, with different versions containing 21 to 35 N-glycosylation sites. In comparison to trimeric glycoproteins found on other human-pathogenic enveloped RNA viruses, coronavirus Spike protein is considerably larger, and totals nearly 700 kDa per trimer. Ectodomains of coronavirus Spike proteins contain an N-terminal domain named S1, which is responsible for binding of receptors on the host cell surface, and a C-terminal S2 domain responsible for fusion. S1 domain of SARS-COV-2 Spike protein is able to bind to ACE2 of host cells. The region of SARS-COV-2 Spike protein S1 domain that recognizes ACE2 is a 25 kDa domain called the receptor binding domain (RBD). When expressed as a stand-alone polypeptide, the RBD can form a functionally folded domain capable of binding ACE2.

In different coronaviruses, Spike proteins may or may not be cleaved during assembly and exocytosis of virions. In most alphacoronaviruses, and in betacoronavirus SARS-COV, the virions harbor uncleaved Spike protein, whereas in virions of some betacoronaviruses, including SARS-COV-2, and in known gammacoronaviruses, Spike protein is found cleaved between the S1 and S2 domains. In these virions, Spike protein is typically cleaved by furin, a Golgi-resident host protease. Accordingly, naturally occurring or “wild-type” amino acid sequence of Spike protein of SARS-COV-2 (which is considered to be the sequence of the first virus SARS-COV-2 isolate, Wuhan-Hu-1), contains a furin cleavage site between S1 and S2 domains. S2 domain of coronavirus Spike proteins contain two heptad repeats, HR1 and HR2, which contain a repetitive heptapeptide characteristic of the formation of coiled-coil that participate in the fusion process. Analysis of sera from COVID-19 patients demonstrates that antibodies are elicited against the Spike protein and can inhibit viral entry into the host cell. The first Cryo-EM structure of SARS-COV-2 Spike protein is described in (9).

A.
“Wild-type” amino acid sequence of
Spike protein of SARS-COV-2-
SEQ ID NO: 1
MFVFLVLLPLVSSQCVNLTTRTQLPPAYTNSFTRGVYYPDKVFRS
SVLHSTQDLFLPFFSNVTWFHAIHVSGTNGTKRFDNPVLPENDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKE
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

An amino acid sequence of a coronavirus Spike protein according to embodiments of the present invention can be a Spike protein sequence from any coronavirus, such as an alphacoronavirus, a betacoronoviurs, a gammacoronovirus, or a deltacoronavirus. Some embodiments described in the present disclosure may refer to a Spike protein of a coronavirus capable of infecting humans (“human coronaviruses”), including, but not limited to, human betacoronaviruses, for example, SARS-COV, MERS-COV, and SARS-COV-2. Some embodiments described in the present disclosure may refer to Spike protein of a coronavirus capable of infecting non-human animals including, but not limited to, BatCoV RaTG13, Bat SARSr-COV ZXC21, Bat SARSr-COV ZC45, BatSARSr-COV WIV1, or other coronaviruses. It is to be understood that a coronavirus Spike protein sequence may be a full or a partial amino acid sequence of a Spike protein, an amino acid sequence of a fragment of a Spike protein, or an amino acid sequence of a variant of a Spike protein, including naturally occurring and artificially generated variants. Some of exemplary variants of Spike protein amino acid sequences are variants found in naturally circulating SARS-COV-2 variants, such as, but not limited to, variants D614G, B.1.1.7 (also known as “UK variant”), B.1.429 (also known as “LA variant”), P1, and B.1.351.

Some embodiments of a coronavirus Spike protein may contain a naturally occurring (or “wild-type”) amino acid sequence of coronavirus Spike proteins or a portion thereof. Some non-limiting examples of such wild-type sequences are: a wild-type amino acid sequence of SI domain of a coronavirus Spike protein; a wild-type amino acid sequence of an RBD domain of a coronavirus Spike protein; or a wild-type amino acid sequence of a coronavirus Spike protein with one or more C-terminal, N-terminal, or middle portions deleted. Some examples of wild-type amino acid sequences of SARS-COV-2 Spike protein are the sequences that contain mutations, in comparison to SEQ ID NO:1, found in naturally occurring SARS-COV-2 strains, which can also be referred to as “variants.”

One such example is a wild-type amino acid sequence of a coronavirus Spike protein having a deletion (in reference to SEQ ID NO:1) of amino acid residues 69-70 and amino acid residue 144, as found in strain SARS-COV-2 VUI 202012/01 in SARS-COV-2 variant lineage B.1.1.7. One more example is a wild-type amino acid sequence of a coronavirus Spike protein having a D to G substitution at amino acid residue 614, (in reference to SEQ ID NO:1), as found in SARS-COV-2 variant D614G. One more example is a wild-type amino acid sequence of a coronavirus Spike protein having the substitutions (in reference to SEQ ID NO:1) S13I, W152C, L452R, and D614G, as found in SARS-COV-2 variant B.1.429. Another example is a wild-type amino acid sequence of a coronavirus Spike protein having substitutions (in reference to SEQ ID NO:1) L18F, T20N, P26S, D138Y, R190S, K417T, E484K, N501Y, D614G, H655Y, T1027I, as found in SARS-COV-2 variant P1. Yet another example is a wild-type amino acid sequence of a coronavirus Spike protein having substitutions (in reference to SEQ ID NO:1) L18F, D80A, D215G, 242-244 del, R246I, K417N, E484K, N501Y, D614G, A701V, as found in SARS-COV-2 variant B.1.351. One more example is a wild-type amino acid sequence of a coronavirus Spike protein having a deletion (in reference to SEQ ID NO:1) of amino acid residues 69-70 and amino acid residue 144, and substitutions (in reference to SEQ ID NO:1) N501Y, A570D, D614G, P681H, T716I, S982A, D1118H, as found in SARS-COV-2 variant B.1.1.7. One more example is a wild-type amino acid sequence of a coronavirus Spike protein having a deletion (in reference to SEQ ID NO:1) of amino acid residues 156-157, and substitutions (in reference to SEQ ID NO:1) T19R, G142D, R158G, L452R, T478K, D614G, P681R, and D950N, as found in SARS-COV-2 variant B.1.617.2.

Some embodiments of coronavirus Spike proteins may contain artificially modified amino acid sequences of coronavirus Spike proteins or portion thereof. In some non-limiting examples, artificially modified amino acid sequences may contain one or more features of the wild-type amino acid sequences of a coronavirus Spike protein sequences, such as, but not limited to, those discussed in the present disclosure. In some exemplary embodiments, the features of the wild-type amino acid sequences of a coronavirus Spike protein sequences may be combined in ways that are not found naturally occurring sequence. For example, an artificially modified amino acid sequence of SARS-COV-2 Spike protein or a portion thereof may include one or more features from each of two or more naturally circulating SARS-COV-2 variants, such as, but not limited to, variants D614G, B.1.1.7, B.1.429, and B.1.351.

Some other non-limiting examples of such artificially modified sequences are: an artificially modified amino acid sequence of SI domain of a coronavirus Spike protein; an artificially modified amino acid sequence of an RBD domain of a coronavirus Spike protein; or an artificially modified amino acid sequence of a coronavirus Spike protein with one or more C-terminal, N-terminal, or middle portions deleted, such as an artificially modified amino acid sequence of a coronavirus Spike protein with a C-terminal deletion encompassing the HR2 amino acid sequence. Artificially modified amino acid sequences of coronavirus Spike proteins may contain various amino acid modifications, as compared wild-type sequences. For example, an artificially modified amino acid sequence of a coronavirus Spike protein may contain mutations removing or adding glycosylation sites. In another example, an artificially modified amino acid sequence of a coronavirus Spike protein may contain one or more mutations eliminating a protease recognition site, such as furin recognition site.

In another example, an artificially modified amino acid sequence of a coronavirus Spike protein may contain one or more mutations affecting a conformation of a Spike domain, such as mutations stabilizing a Spike domain in a pre-fusion conformation. SEQ ID NO:2, described in (47), is an artificially modified SARS-COV-2 Spike protein sequence termed “S-2P” with a furin cleavage site PRAR sequence mutated to alanine (amino acid residue 667 in SEQ ID NOs 1 and 2) and proline substitutions at amino acid residues 968 and 969 of SEQ ID NO:1. S-2P is stabilized in a pre-fusion conformation.

FIG. 1A schematically illustrates pre-fusion-stabilized SARS-COV-2 Spike protein and a model of the trimeric pre-fusion conformation. SEQ ID NO:3, described in (30), is an artificially modified SARS-COV-2 Spike protein sequence (“HexaPro”) with six proline substitutions: F817P, A892P, A899P, A942P (all denoted with respect to SEQ ID NO:1), and proline substitutions at amino acid residues 968 and 969 of SEQ ID NO:1.

B.
Artificially modified SARS-COV-2
Spike protein sequence mutation
of PRAR furin cleavage site to alanine
and proline substitutions are shown in bold
SEQ ID NO: 2
MFMPSSFSYSSWATCWLLCCLIILAKATMFVFLVLLPLVSSQCVN
LTTRTQLPPAYTNSFTRGVYYPDKVFRSSVLHSTQDLFLPFFSNV
TWFHAIHVSGINGTKRFDNPVLPENDGVYFASTEKSNIIRGWIFG
TTLDSKTQSLLIVNNATNVVIKVCEFQFCNDPFLGVYYHKNNKSW
MESEFRVYSSANNCTFEYVSQPFLMDLEGKQGNEKNLREFVFKNI
DGYFKIYSKHTPINLVRDLPQGFSALEPLVDLPIGINITRFQTLL
ALHRSYLTPGDSSSGWTAGAAAYYVGYLQPRTFLLKYNENGTITD
AVDCALDPLSETKCTLKSFTVEKGIYQTSNERVQPTESIVRFPNI
TNLCPFGEVENATRFASVYAWNRKRISNCVADYSVLYNSASFSTF
KCYGVSPTKLNDLCFTNVYADSFVIRGDEVRQIAPGQTGKIADYN
YKLPDDFTGCVIAWNSNNLDSKVGGNYNYLYRLFRKSNLKPFERD
ISTEIYQAGSTPCNGVEGENCYFPLQSYGFQPTNGVGYQPYRVVV
LSFELLHAPATVCGPKKSTNLVKNKCVNFNFNGLTGTGVLTESNK
KELPFQQFGRDIADTTDAVRDPQTLEILDITPCSFGGVSVITPGT
NTSNQVAVLYQDVNCTEVPVAIHADQLTPTWRVYSTGSNVFQTRA
GCLIGAEHVNNSYECDIPIGAGICASYQTQTNSPASVASQSIIAY
TMSLGAENSVAYSNNSIAIPTNFTISVTTEILPVSMTKTSVDCTM
YICGDSTECSNLLLQYGSFCTQLNRALTGIAVEQDKNTQEVFAQV
KQIYKTPPIKDFGGFNFSQILPDPSKPSKRSFIEDLLENKVTLAD
AGFIKQYGDCLGDIAARDLICAQKFNGLTVLPPLLTDEMIAQYTS
ALLAGTITSGWTFGAGAALQIPFAMQMAYRFNGIGVTQNVLYENQ
KLIANQFNSAIGKIQDSLSSTASALGKLQDVVNQNAQALNTLVKQ
LSSNFGAISSVLNDILSRLDPPEAEVQIDRLITGRLQSLQTYVTQ
QLIRAAEIRASANLAATKMSECVLGQSKRVDFCGKGYHLMSFPQS
APHGVVFLHVTYVPAQEKNFTTAPAICHDGKAHFPREGVFVSNGT
HWFVTQRNFYEPQIITTDNTFVSGNCDVVIGIVNNTVYDPLQPEL
DSFKEELDKYFKNHTSPDVDLGDISGINASVVNIQKEIDRLNEVA
KNLNESLIDLQELGKYEQYIKWPSGRLVPRGSPGSGYIPEAPRDG
QAYVRKDGEWVLLSTFLGHHHHHH;
C.
Artificially modified SARS-COV-2
Spike protein sequence “HexaPro”-
proline substitutions are shown in bold
SEQ ID NO: 3
MFVFLVLLPLVSSQCVNLTTRTQLPPAYTNSFTRGVYYPDKVERS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTELLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLENKVTLADAGFIKQYGDCLGDIAARDLICAQKE
NGLTVLPPLLTDEMIAQYTSALLAGTITSGWTFGAGPALQIPFPM
QMAYRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTELGRSLEVLFQGPGHHHHHHHHSAW
SHPQFEKGGGSGGGGSGGSAWSHPQFEK;

In some embodiments, the amino acid sequence of a Spike protein of a coronavirus is an amino acid sequence with at least 80%, at least 85%, at least 90%, at least 95%, at least 96%, at least 97%, at least 98%, or at least 99% sequence identity to a wild-type or artificially modified amino acid sequence of SARS-COV-2 Spike protein amino acid sequence. In some embodiments, the amino acid sequence of a Spike protein of a coronavirus included in a fusion protein as provided herein is an amino acid sequence with at least 80%, at least 85%, at least 90%, at least 95%, at least 96%, at least 97%, at least 98%, or at least 99% sequence identity to a portion of the amino acid sequence of wild-type or artificially modified SARS-COV-2 Spike protein amino acid sequence. In some instances, the Spike protein of a coronavirus is a conservatively modified variant Spike protein comprising one or more amino acid residue substitutions. In some instances, the Spike protein of a coronavirus included in a fusion protein as provided herein comprises a deletion of one or more amino acid residues at the C-terminal, N-terminal, and/or middle portion of the protein. In some instances, the deletion may comprise a one or more consecutive amino acid residues. In some instances, the deletion may comprise a one or more non-consecutive amino acid residues. In some instances, the Spike protein may comprise a deletion of 1, 2, 3, 4, 5, 6, 7, 8, 9, or 10 amino acid residues. In some instances, the Spike protein may comprise a deletion of 10, 15, 20, 25, 30, 35, 40, 45, 50, 55, 60, 65, 70, 75, 80, 85, 90, 95, or 100 amino acid residues, such as deletions of 10-15, 15-30, 25-50, 10-50, or 50-100 amino acid residues.

### II. HYDROGEN DEUTERIUM EXCHANGE (HDX)

Hydrogen deuterium exchange (HDX) is discussed, for example, in (23). HDX is a solution-based technique for analyzing conformational flexibility of polypeptide or protein molecules. HDX experiments involve solutions of polypeptides or proteins in buffered D20, in which cations of deuterium exchange with labile protons in a polypeptide chain in a time-dependent fashion. Deuterium exchange is measured by various techniques, and the resulting data is processed by computer-based methods to infer information about the dynamics of the polypeptide molecule. HDX coupled with mass spectrometry (HDX-MS) is used for structural and dynamic studies of protein molecules and their interactions with ligands.

In HDX-MS, protein molecules are incubated with deuterated solvent at various time points, usually under native conditions, followed by quenching with a cold acid solution, which may also contain denaturants to aid in subsequent proteolysis. Quenched samples are then subjected to proteolysis, usually by passing them over an immobilized protease column. The resulting proteolytic fragments are captured, separated, and subjected to mass spectrometry detection to quantitate the levels of deuterium incorporation (encompassed by the expression “deuterium incorporation data”) into each proteolytic peptide. HDX can also be performed in reverse, on a deuterated sample measuring the incorporation of protons.

Mass spectrometry data for each peptide provides spectrum of mass-to-charge (m/z) values, which are distributed on a Gaussian, reflecting a range of deuterated species of the same peptide. The distribution of masses for a given exchange time point is a convolution of both the natural isotopic abundance of the atoms in the peptide (e.g. carbon 12 and 13, or nitrogen 14 and 15), and the number of hydrogen atoms that have exchanged for deuterium. This observed distribution of masses is often called the “mass envelope” or the “isotopic envelope”. HDX-MS data may be followed for each peptide across various time points under different conditions (an approach termed “differential HDX”). A maximally deuterated sample, termed Dmax control, is used for correction of back exchange. In a typical hydrogen exchange experiment, the sample is quenched into an H₂O solution. During the resulting sample injection, digestion, and separation, the deuterium atoms that have incorporated into the protein can exchange with the protons in the quench solution, albeit at a slow rate. This results signal attenuation, which is frequently called “back exchange.” To correct for back exchange, a sample with maximal deuteration uptake is created and subjected to the same experimental conditions. This experiment allows for the quantification of the deuterium loss due to back exchange.

HDX-MS data are analyzed using automated computer software, and the data from all overlapping peptides are consolidated to individual amino acid values using a residue averaging approach. HDX-MS data can be visualized in various ways, such as deuterium uptake plots and sequence coverage heat maps. HDX-MS data can be used together from the data obtained by other structural analysis techniques, for example, by coupling the solution-phase solvent exchange measurements from HDX-MS with static structures derived by X-ray crystallography, cryoEM and NMR spectroscopy, to inform the understanding of protein structure.

A labile hydrogen that is bonded to nitrogen, oxygen, or sulfur atom in a polypeptide molecule found in an aqueous solution can exchange with deuterium from the solvent. The exchange reaction is either acid or base-catalyzed and proceeds with the rate constant kₑₓ=k_{H}[H⁺]+k_{OH}[OH^{-]+k}_{H2O}[H₂O], wherein k_{H}, k_{OH} and k_{H2O} are the rate constants for the acid, base-catalyzed, and water-catalyzed exchange, respectively, as shown in the equation. The upper limit of kₑₓ—the intrinsic exchange rate—of the amide hydrogen atoms in a polypeptide molecule in an aqueous solution has been defined; it represents the exchange rate at which the amide hydrogen atoms can readily exchange with solvent deuterium, if they are accessible to the solvent and free from intra-molecular or inter-molecular hydrogen bonding, as occurs in a-helices, b-sheets, and protein-protein interactions (23). As stated in (23), “as backbone amides form hydrogen bonds with backbone carbonyl oxygen in secondary structural elements, the structure of a folded protein may protect amides from undergoing HDX at the intrinsic rate by as great as eight orders of magnitude. The fold change reduction in amide hydrogen exchange is termed the “protection factor” (PF). As a protein experiences structural fluctuations, local energy barriers are overcome, which results in the transient exposure of the labile hydrogen. At this point, labile hydrogen can exchange with solvent hydrogen with a rate constant kₑₓ=k_{ch}, where k_{ch} is the rate constant of intrinsic HDX exchange rate of an amide proton in the open or unfolded state.”

In a folded polypeptide molecule, an amide proton exchanges with solvent deuterium through structural changes termed “opening and closing events,” which proceed with exchange rate constants kop and kcl, respectively. The measured rates of HDX for a folded polypeptide molecules depend by the rate of the structural changes and intrinsic HDX kinetics. The established kinetic model for hydrogen exchange during HDX has two regimes termed EX2 and EX1. Hydrogen exchange follows EX2 regime when kcl>>kch, with the refolding rate much faster than the intrinsic exchange rate of the amide hydrogens, resulting in the observation of one isotopic or mass envelope (unimodal Gaussian distribution of m/z values) throughout the labeling time of an HDX-MS experiment.

The HDX kinetics for native proteins usually follows EX2 kinetics at near-physiological conditions (base-catalyzed, pH 5-8) and in the absence of chaotropes. EX1 regime occurs when kch>>kcl, with a refolding event occurring sufficiently slowly to allow complete deuterium exchange of backbone amide hydrogens within the unfolding region (in other words, when the rates of hydrogen bond closing are much slower than the intrinsic chemistry of the exchange process). Under EXI conditions, if an opening or unfolding event of a polypeptide molecule involves more than one slowly refolding amide, then deuterium exchange occurs simultaneously at these amides, and bimodal distribution of m/z values is observed throughout the labeling time of an HDX-MS experiment. In this case, the lower mass envelope correspond to molecules that have not yet exchanged (not yet unfolded) and the higher mass envelope corresponds to molecules that have undergone exchange (molecules that have unfolded).

In EXI scenario, the heavier mass distribution will increase in intensity at the expense of the lighter one over the observed time period. EX1 pattern is usually observed when a protein is exposed to strongly denaturing conditions. In some instances, bimodal distribution of m/z values can also be observed in an HDX-MS experiment under physiological conditions due to conformational heterogeneity of the polypeptide molecule. In this scenario, bimodal distribution indicates the presence of two different polypeptide conformations that interconvert slowly on the timescale of an HDX experiment experiment: one where the amides are more accessible to exchange compared to the other. In such a situation, the two peaks of the bimodal mass distribution retain their relative intensities, increasing in average mass over time.

### III. HDX-MS ANALYSIS TO DETECT CONFORMATIONS OF SARS-COV-2 SPIKE PROTEIN

### A. HDX-MS-Based Methods of Detecting Conformations of SARS-Cov-2 Spike Protein

Included among the embodiments of the present invention are methods that use HDX-MS analysis to detect conformations of SARS-COV-2 Spike protein. When aqueous solutions of SARS-COV-2 Spike protein were analyzed by HDX-MS, a number of peptides of SARS-COV-2 Spike protein exhibited bimodal behavior with both isotopic envelopes increasing in mass over time, thus indicating the presence of two different conformations. The peptides SARS-COV-2 Spike protein that exhibited the above bimodal behavior in HDX-MS studies can be referred to as “bimodal peptides” in the present disclosure. In the bimodal peptides, the isotopic envelope corresponding to lower degree of the deuterium exchange (“less-exchanged”) was consistent with the known, cryoEM-determined, structure of SARS-COV-2 Spike protein trimer (“first conformation,” “conformation A,” or “A state”), while the isotopic envelope corresponding to higher degree of deuterium exchange (“more-exchanged”) indicated a presence of a second, previously unknown conformation of SARS-COV-2 Spike protein trimer (“second conformation,” “conformation B,” or “B state”). It can be said that the HDX-MS analysis of the bimodal peptides showed two different sets of deuterium incorporation data in the first conformation and in the second conformation of the SARS-COV-2 Spike protein. The newly discovered second conformation of the SARS-COV-2 Spike protein included solvent-accessible regions (detectable by HDX-MS as bimodal peptides) that were not present in the first conformation.

The present disclosure describes methods of detecting a second conformation of SARS-COV-2 Spike protein by HDX-MS analysis. In an exemplary embodiment, the HDX-MS analysis involves incubating an aqueous solution of the SARS-COV-2 Spike protein with D20, thereby generating a sample of partially deuterated SARS-COV-2 Spike protein, quenching the sample of the partially deuterated SARS-COV-2 Spike protein, for example, by adding a solution of cold acid, subjecting the quenched sample of the partially deuterated SARS-COV-2 Spike protein to protease digestion (for example, by passing it through a chromatography column with an immobilized protease), thereby generating a mixture of partially deuterated proteolytic peptides of the SARS-COV-2 Spike protein, and analyzing the mixture by MS, thus generating HDX-MS data.

The HDX-MS data for each partially deuterated proteolytic peptide, including bimodal peptides, can then be computationally converted into a spectrum of mass-to-charge (m/z) ratios (such spectra are encompassed by the expression “deuterium incorporation data”). The m/z spectra for one or more of the bimodal peptides can be computationally analyzed to determine the conformation (that is, a first or a second conformation) of the SARS-COV-2 Spike protein in the aqueous solution subjected to HDX-MS analysis. For example, the computational analysis of the m/z spectra for the bimodal peptides can indicate a distribution of the SARS-COV-2 Spike protein in the aqueous solution between the first conformation and the second conformation.

Exemplary computational analysis may involve fitting a sum of two distribution functions (e.g., Gaussian distributions, multinomial (e.g., binomial) distributions, exponential distributions, Poisson distributions, etc.) to the mass spectrum for a particular bimodal peptide and calculating an area under each of the two distribution functions to determine proportions of the first conformation and the second conformation the SARS-COV-2 Spike protein in the aqueous solution subjected to HDX-MS analysis. Gaussian functions will be used as an example. Each Gaussian function can correspond to a different conformation. For example, the Gaussian with a maximum at higher m/z of the mass spectrum can correspond to the second conformation (conformation B) of the bimodal peptide (the second conformation being more solvent exposed, thus having higher average hydrogen solvent exchange at that time point, and, consequently, deuterium incorporation, resulting in higher m/z peak of the mass spectrum) while the Gaussian with a maximum at lower m/z of the mass spectrum can correspond to the first conformation (conformation A) of the bimodal peptide (the first conformation being less solvent exposed, thus having lower average hydrogen solvent exchange, and, consequently, deuterium incorporation, resulting in lower m/z peak of the mass spectrum). The peaks and widths of the two Gaussians can be varied (e.g., as a mixture ratio of the two Gaussians) until a best fit to the bimodal spectrum is determined. Then, an amount of each conformation can be determined from the properties of the two Gaussians.

The area under each Gaussian (or some scalar multiple of such area) can provide the relative amount for the respective conformation. In other words, exemplary computational analysis involves creating a mixture model (“mixture modeling”) with two Gaussian distributions, each representing a subpopulation of the first and the second conformations in an overall population of a bimodal peptide of a SARS-COV-2 Spike protein. Mixture modelling allows one to calculate proportions of conformations A and B in in an overall population of a bimodal peptide of a SARS-COV-2 Spike protein. The process of fitting two Gaussians has six parameters: the height of each Gaussian, the width of each Gaussian, and the relative position of each Gaussian. When a single hydrogen exchange time point is fit, then all six parameters can be optimized to best fit the data. When fitting incubation kinetics (a pulsed-labeling HDX experiment at different temperature incubation times) then it is assumed that each distribution would have the same width and position at all time points, with only the heights changing. This results in four parameters (both widths and positions) that are globally fit to all data, and two parameters (both heights) fit individually for each distribution.

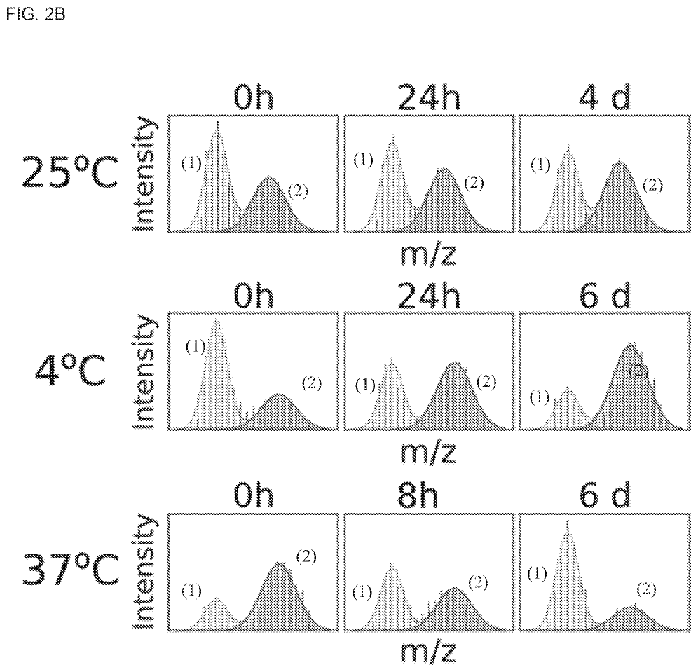

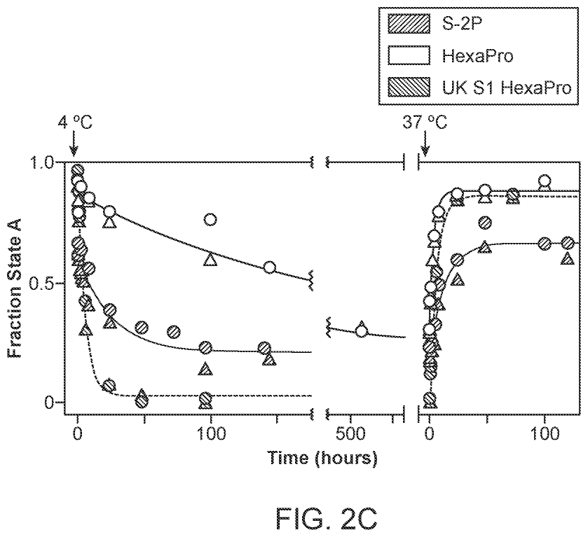

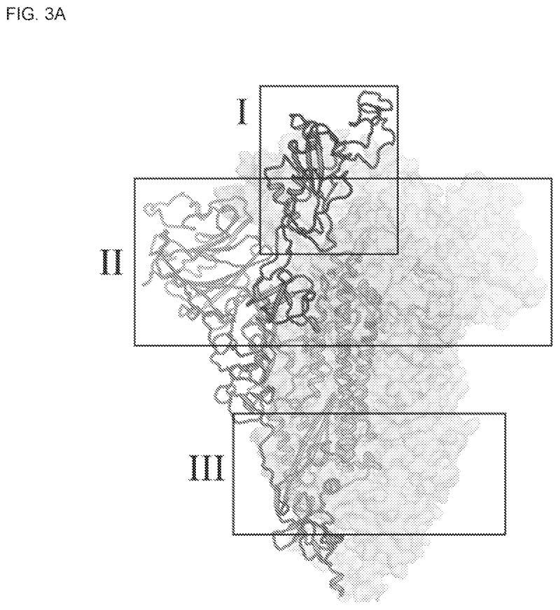

FIG. 2A illustrates mixture modeling by showing two Gaussians fit to the mass spectra of two different bimodal peptides of a SARS-COV-2 Spike protein. The two Gaussians are shown underneath state A (1) and state B (2).

### B. Bimodal Peptides of SARS-Cov-2 Spike Protein

The bimodal peptides of a SARS-COV-2 Spike protein, meaning the peptides for which bimodal m/z spectra were detected by HDX-MS analysis, include (in reference to SEQ ID NO:1, although other SARS-COV-2 Spike protein sequences can be used for reference), the peptides with amino acid sequences corresponding to (or homologous to, for example, having at least 90% homology) to amino acid residues 291-300 of SEQ ID NO:1, amino acid residues 291-303 of SEQ ID NO:1, amino acid residues 553-568 of SEQ ID NO:1, amino acid residues 626-636 of SEQ ID NO:1, amino acid residues 662-673 of SEQ ID NO:1, amino acid residues 878-901 of SEQ ID NO: 1, amino acid residues 878-902 of SEQ ID NO:1, amino acid residues 904-916 of SEQ ID NO: 1, amino acid residues 962-967 of SEQ ID NO:1, C, amino acid residues 982-1001 of SEQ ID NO:1, amino acid residues 978-1001 of SEQ ID NO: 1, amino acid residues 982-1001 of SEQ ID NO: 1, amino acid residues 1002-1024 of SEQ ID NO: 1, amino acid residues 1146-1166 of SEQ ID NO:1, amino acid residues 1179-1186 of SEQ ID NO:1, amino acid residues 1187-1197 of SEQ ID NO:1. It is noted that the above peptides are proteolytic fragments detected during HDX-MS analysis. Since partial protease digestion was used to generate proteolytic peptide, slightly different proteolytic peptides were detected for different SARS-COV-2 Spike proteins (which is not uncommon).

For the bimodal peptides detected by HDX-MS analysis, the difference between the first conformation and the second conformation of the SARS-COV-2 Spike protein can be characterized as having one or more of the above peptides more solvent-exposed (and thus exhibiting a higher degree of the deuterium exchange, or heavier mass envelope) in the second conformation and less solvent-exposed (or more buried, and thus exhibiting a lower degree of the deuterium exchange, or lighter mass envelope) in the first conformation. For example, the second conformation of the SARS-COV-2 Spike protein comprises solvent-exposed amino acid residues in the regions corresponding an inter-promoter interface of a trimer in the first conformation of the SARS-COV-2 Spike protein, the solvent-exposed amino acid residues located in SARS-COV-2 Spike protein acid sequence in regions corresponding to (or homologous to, for example, with at least 90% homology) amino acid residues 870-916, 553-574, 662-673,962-1024, 1146-1166, 1187-1196, 962-1024, 1146-1166, and 1187-1196 of SEQ ID NO:1.

In another example, the second conformation of the SARS-COV-2 Spike protein comprises solvent-exposed amino acid residues in the regions corresponding to an interface between N-terminal domain and second S1 subdomain (SD2) in the first conformation of the SARS-COV-2 Spike protein, the solvent-exposed amino acid residues located in SARS-COV-2 Spike protein acid sequence in regions corresponding to (or homologous to, for example, with at least 90% homology) amino acid residues 291-305 and 626-636 of SEQ ID NO:1. The second conformation of the SARS-COV-2 Spike protein can also be described as having a binding site for 3A3 antibody in a region corresponding to (or homologous to, for example, with at least 90% homology) amino acid residues 978-1001 of SEQ ID NO:1. The HDX-MS analysis found that the binding site for 3A3 antibody was occluded from solvent in a complex of the second conformation of SARS-COV-2 Spike protein and 3A3 antibody.

### C. Methods of Detecting Ligands Capable of Stabilizing a First or a Second Conformation of a SARS-Cov-2 Spike Protein

Also included among the embodiments of the present invention are methods of determining if a ligand is capable of stabilizing a first or a second conformation of a SARS-COV-2 Spike protein. It is envisioned that such methods may be useful in various contexts, including, but not limited to, laboratory research and drug design. For instance, ligands (including, but not limited to, small molecules and antibodies) that are capable of stabilizing a second conformation of SARS-COV-2 Spike protein, may “trap” the protein in the second conformation, thereby diminishing the ability of SARS-COV-2 Spike protein to facilitate SARS-COV-2 infection. Accordingly, ligands that are capable of stabilizing a second conformation of SARS-COV-2 Spike protein may be drug candidates. Exemplary embodiments of methods of determining if a ligand is capable of stabilizing a first or a second conformation of a SARS-COV-2 Spike protein involve performing HDX-MS analysis of the aqueous solution of the SARS-COV-2 Spike protein and a ligand. The HDX-MS analysis, performed as described elsewhere in the present disclosure, generates HDX-MS data that is computationally converted into deuterium incorporation data for one or more of the bimodal peptides. The deuterium incorporation data for one or more bimodal peptides can be, in turn, computationally analyzed (e.g., using a mixture model of the resulting spectrum) to determine a distribution (e.g., mixture ratio of the two Gaussians) between the first conformation and a second conformation of the SARS-COV-2 Spike protein in the aqueous solution in the presence of the ligand. The distribution in the presence of the ligand can be compared to the distribution in the absence of the ligand (the latter can be determined in by HDX-MS analysis as a control or derived from already available data). The ligand is considered to be capable of stabilizing the first conformation of the SARS-COV-2 Spike protein when a proportion of the SARS-COV-2 Spike found in the first conformation is increased in presence of the ligand, as compared to absence of the ligand. The ligand is considered to be capable of stabilizing the second conformation of the SARS-COV-2 Spike protein when a proportion of the SARS-COV-2 Spike found in the second conformation is increased in the presence of the ligand, as compared to the absence of the ligand. It is to be understood that methods of identifying ligand is capable of stabilizing a first or a second conformation of a SARS-COV-2 Spike protein need not be conducted using laboratory techniques. Such methods may be also conducted in silico using computational methods and systems described elsewhere in the present disclosure.

### D. Methods of Detecting Binding of a Ligand to a Second Conformation of a SARS-COV-2 Spike Protein

Methods of identifying if a ligand is capable of binding to a second conformation of a SARS-COV-2 Spike protein, which can also be described as methods of detecting binding of a ligand to a second conformation of SARS-COV-2 Spike protein, are also envisioned and included among the embodiments of the present invention. Such methods may be useful in various contexts, including, but not limited to, laboratory research and drug design. For instance, ligands (including, but not limited to, small molecules and antibodies) that are capable of binding to a second conformation of a SARS-COV-2 Spike protein may be potential drug candidates against SARS-COV-2 infection. Methods of identifying a ligand capable of binding to a second conformation of a SARS-COV-2 Spike protein may involve performing HDX-MS analysis of the aqueous solution of the SARS-COV-2 Spike protein and a ligand. The HDX-MS analysis, performed as described elsewhere in the present disclosure, generates HDX-MS data that is computationally converted into deuterium incorporation data for one or more of the bimodal peptides. The deuterium incorporation data for one or more bimodal peptides can be, in turn, computationally analyzed (e.g., using a mixture model of the resulting spectrum) to determine a distribution (e.g., mixture ratio of the two Gaussians) between a first conformation and a second conformation of the SARS-COV-2 Spike protein in the aqueous solution in the presence of the ligand. The distribution in the presence of the ligand can be compared to the distribution in the absence of the ligand (the latter can be determined in by HDX-MS analysis as a control or derived from already available data) to detect if any of bimodal peptides of the SARS-COV-2 Spike protein became less solvent exposed after exposure to the ligand. If decreased solvent exposure of one or more of the bimodal peptides is observed, it means that the ligand is capable of binding to the second conformation of the SARS-COV-2 Spike protein and shielding the previously solvent-exposed bimodal peptide from solvent exposure. It is to be understood that methods of identifying if a ligand capable of binding to a second conformation of SARS-COV-2 Spike protein need not be conducted using laboratory techniques. Such methods may be also conducted in silico using computational methods and systems described elsewhere in the present disclosure.

### E. Methods of Producing and or Stabilizing a Second Conformation of a SARS-Cov-2 Spike Protein

Methods of producing and/or stabilizing a second conformation of a SARS-COV-2 Spike protein are also envisioned and included among the embodiments of the present invention. Some embodiments of methods of producing and/or stabilizing a second conformation of a SARS-COV-2 Spike protein may involve contacting a starting population of SARS-COV-2 Spike protein molecules with one or more ligands capable of stabilizing a second conformation of SARS-COV-2 Spike protein and/or effecting a conversion of SARS-COV-2 Spike protein molecules from a first conformation into a second conformation. Bringing SARS-COV-2 Spike protein molecules in contact with the one or more ligands may occur in aqueous solution. In some examples, upon binding to SARS-COV-2 Spike protein molecule, the one or more ligands stabilize SARS-COV-2 Spike protein molecule in a second conformation, thereby resulting in a population of SARS-COV-2 Spike protein molecules that has a larger proportion of SARS-COV-2 Spike protein molecules in a second conformation than the starting population of SARS-COV-2 Spike protein molecules. Non-limiting examples of ligands that are capable of stabilizing SARS-COV-2 Spike protein molecule in a second conformation are ACE2 and 3A3 antibody. Some embodiments of methods of producing and/or stabilizing a second conformation of a SARS-COV-2 Spike protein may involve exposing a SARS-COV-2 Spike protein, for example, in an aqueous solution, to various conditions, such as temperatures, pressures, pH, ionic strengths, surfactants, etc., that stabilize a second conformation of a SARS-COV-2 Spike protein and/or effect conversion of a first conformation of a SARS-COV-2 Spike protein into a second conformation. Some embodiments of methods of producing and/or stabilizing a second conformation of a SARS-COV-2 Spike protein may involve using various modifications of SARS-COV-2 Spike protein molecule, such as introduction of amino acid mutations (for example, substitutions) or posttranslational modifications, into a SARS-COV-2 Spike protein molecule.

Methods of producing and/or stabilizing a second conformation of a SARS-COV-2 Spike protein may be useful in various contexts, including, but not limited to, scientific research or therapeutic applications. For instance, ligands (including, but not limited to, small molecules and antibodies) that are capable of stabilizing a second conformation of a SARS-COV-2 Spike protein and/or converting SARS-COV-2 Spike protein from first conformation into second conformation may be used to treat and/or prevent SARS-COV-2 infection. Methods of producing and/or stabilizing a second conformation of a SARS-COV-2 Spike protein may involve performing HDX-MS analysis as described elsewhere in the present disclosure. The resulting HDX-MS data that is computationally converted into deuterium incorporation data for one or more of the bimodal peptides. The deuterium incorporation data for one or more bimodal peptides can be, in turn, computationally analyzed (e.g., using a mixture model of the resulting spectrum) to determine a distribution (e.g., mixture ratio of the two Gaussians) between the first conformation and the second conformation of the SARS-COV-2 Spike protein in the aqueous solution. In one example, the distribution in the presence of the ligand can be compared to the distribution in the absence of the ligand (control distribution). In another example, the distribution after exposure to certain conditions can be compared to the distribution in the absence of the ligand (control distribution). In one more example, the distribution of a SARS-COV-2 Spike protein having one or more mutations or posttranslational modifications can be compared to the distribution of a SARS-COV-2 Spike protein without such one or more mutations or posttranslational modifications (control distribution). In the above examples or other situations, control distribution can be determined in by HDX-MS analysis as a control experiments, or derived from already available data. If, upon comparison of the distributions, an increase in the second conformation of one or more of the bimodal peptides is observed, it means that a method produced and/or stabilized the second conformation of SARS-COV-2 Spike protein. It is to be understood that identification of ligands, conditions, or modifications a ligand capable of binding to a second conformation of SARS-COV-2 Spike protein may be identified using in silico using computational methods and systems described elsewhere in the present disclosure.

### F. Methods Utilizing Second Conformation SARS-Cov-2 Spike Protein

Also included among the embodiments of the present invention are methods that utilize aqueous solutions comprising SARS-COV-2 Spike protein in the second conformation (state B). Such methods may be useful in various contexts, including, but not limited to, laboratory research and drug design, such as for identification of ligands (including, but not limited to, small molecules and antibodies) that are capable of binding to a second conformation of a SARS-COV-2 Spike protein and may serve as potential drug candidates against SARS-COV-2 infection. Some embodiments of such methods may utilize aqueous solutions of SARS COV 2 Spike protein found in the second conformation or predominantly or substantially in the second conformation may utilize a SARS COV 2 Spike protein found predominantly (at >50%, at least 60%, at least 70%, at least 75%, at least 80%, at least 85%, at least 90%, at least 95%, or at least 99%) in the second conformation. Some other embodiments of such methods may utilize aqueous solutions of SARS-COV-2 Spike protein that have less than 50% SARS-COV-2 Spike protein in a second conformation (for example, but not limited to, 0.01%-49.99%, 0.01%-40%, 0.01%-30%, 0.01%-20%, 0.01%-10%, or 0.01%-1%,). Aqueous solutions comprising SARS-COV-2 Spike protein in can be prepared, for example, using various methods and conditions, such as, but not limited to, incubation at one or more of a particular temperature (for example, approximately 4° C.) and/or for a particular time, that stabilize the second conformation of SARS-COV-2 Spike protein and/or lead to conversion of the SARS-COV-2 Spike protein into the second conformation. In another example, amino acid sequence of the SARS-COV-2 Spike protein can be modified to incorporate amino acid changes that lead to the shift of SARS-COV-2 Spike protein molecules to the second conformation. In one exemplary embodiment, provided is a method of detecting binding of a ligand to a second conformation of a SARS-COV-2 Spike protein. Such a method may involve contacting the ligand with the aqueous solution comprising the SARS-COV-2 Spike protein in the second conformation, and, after the contacting, performing a suitable in vitro analytical method to detect binding of the ligand to SARS-COV-2 Spike protein.

### IV. COMPUTATIONAL METHODS

Envisioned and included among the embodiments of the present invention are methods that involve computational (in silico) selection and/or identification of ligands capable of binding to a second conformation of SARS-COV-2 Spike protein. Instead of actually combining potential ligands with SARS-COV-2 Spike protein in a second conformation in a laboratory setting and measuring experimental results, computational methods use computers to simulate (model in silico) or characterize molecular interactions between at least one ligand and SARS-COV-2 Spike protein molecule, or a portion of SARS-COV-2 Spike protein (for example, a potential binding site for a ligand). The use of computational methods to assess molecular combinations and interactions may be performed as one or more stages of rational drug design, or in other contexts.

### A. Rational Drug Design

Rational drug design may incorporate the use of any of a number of computational components ranging from computational modeling of target-ligand molecular interactions and combinations to lead optimization to computational prediction of desired drug-like biological properties. Rational drug design may incorporate the use of any of a number of computational components ranging from computational modeling of target-ligand molecular interactions and combinations to lead optimization to computational prediction of desired drug-like biological properties. The use of computational modeling in the context of rational drug design has been largely motivated by a desire both to reduce the required time and to improve the focus and efficiency of drug research and development, by avoiding often time consuming and costly efforts in biological “wet” lab testing and the like. In the context of the present disclosure, SARS-COV-2 Spike protein molecule, or its portion, such as a potential binding site for a drug, can serve as a drug target in the drug design process. Structure-based rational drug design can utilize a three-dimensional model of the structure for the target. For target proteins or nucleic acids, such structures may be the result of cryoEM, X-ray crystallography, NMR or other measurement procedures or may result from homology modeling, analysis of protein motifs and conserved domains, and/or computational modeling of protein folding or the nucleic acid equivalent. For example, in the context of the present disclosure, a structure of SARS-COV-2 Spike protein molecule or its portion, such as a potential ligand binding site, determined by cryoEM (as well as structures determined by other methods, or fully modeled) and supplemented by solved accessibility data derived from HDX-MS analysis can be used as a structure of SARS-COV-2 Spike protein molecule.

B. Using Data Derived from HDX-MS Analysis in Computational Modeling

Solvent accessibility data derived from HDX-MS analysis as described elsewhere in the present disclosure can be incorporated into computational modeling of a structure of SARS-COV-2 Spike protein molecule or its fragments. In exemplary approach, computation model of a full or partial structure of SARS-COV-2 Spike protein molecule in a second confirmation would model as solvent-exposed the trimeric interface in the regions observed bimodal peptides. Computational models of these regions may then be used in computational drug design and computational docking approaches. Until the discoveries by the inventors described in the present disclosure, trimeric interfaces of SARS-COV-2 Spike protein molecule were treated as solvent-protected and thus not targeted by computational drug design and docking approaches. The unexpected findings described in the present disclosure advantageously provide new potential binding sites for computation drug design and computational docking. Solvent accessibility data derived from HDX-MS analysis may also be used in integrative structural biology modeling approaches that computationally generate an ensemble of conformations of SARS-COV-2 Spike protein molecule or its fragments, and then attempt to choose the set and populations of those conformations that best explain the observed experimental data. In additional to HDX-MS data, various computational modeling approaches may use experimental data obtained by one or more other structural biology methods, such as, but not limited to, X-ray crystallography, small angle X-ray scattering (SAXS), or cryoEM.

### C. In Silico Screening of Ligand Libraries and Other Ligand Discovery Methods

Computational modeling of target-ligand molecular combinations, in the context of rational drug design or other contexts, may involve the large-scale in silico screening of ligand libraries, such as small-molecule libraries, whether the libraries are virtually generated and stored as one or more compound structural databases or constructed via combinatorial chemistry and organic synthesis, using computational methods to rank a selected subset of ligands based on computational prediction of bioactivity (or an equivalent measure) with respect to the intended target molecule. Fragment-based drug discovery (FBDD), discussed is another tool for discovering ligands, including leads for drug development. FBDD first identifies starting points: low-molecular-weight ligands (˜150 Da) (fragments) that bind to a target. The fragments may bind to the target with the very low affinity. The identified fragments may be them grown or combined to produce leads with higher affinity. The three-dimensional binding mode of the fragments may be determined in silico and/or experimentally, using X-ray crystallography or NMR spectroscopy, and is used to facilitate their optimization into leads with higher activity. FBDD can be combined with screening.

In the context of the present disclosure, the target molecule may be SARS-COV-2 Spike protein molecule, or a portion of SARS-COV-2 Spike protein molecule, that incorporates that data generated by HDX-MS and described in the present disclosure. For example, a target molecule may be a SARS-COV-2 Spike protein trimer in an “open trimer” conformation described elsewhere in the present disclosure, or a portion of the SARS-COV-2 Spike protein trimer in the “open trimer” conformation. In another example, a target molecule may be a SARS-COV-2 Spike protein monomer (protomer) incorporating solvent accessibility data generated by HDX-MS analysis for the bimodal peptides. In yet another example, a portion of a SARS-COV-2 Spike protein trimer or monomer comprising one or more of the bimodal peptides (such as, but not limited, to those located on the trimer interface) and incorporating solvent accessibility data generated by HDX-MS these one or more bimodal peptides. An exemplary model of SARS-COV-2 Spike protein molecule or a portion of SARS-COV-2 Spike protein molecule used in the computational modelling envisioned by the present disclosure in a second conformation includes one or more bimodal peptides in a solvent accessible state (as found in the second conformation), as detected by HDX-MS and described elsewhere in the present disclosure.

In one exemplary embodiment, a method of identifying a ligand capable of binding to SARS-COV-2 Spike protein may involve screening in silico a ligand library for candidate ligands capable of binding to a first conformation of the SARS-COV-2 Spike protein, a second conformation of the SARS-COV-2 Spike protein, or both to the first and the second conformation of the SARS-COV-2 Spike protein, wherein three-dimensional models of the first conformation and the second conformation of the SARS-COV-2 Spike protein are computationally derived and incorporate solvent accessibility information based on deuterium incorporation obtained by HDX-MS analysis. In another example, a method of identifying a ligand capable of binding to SARS-COV-2 Spike protein may involve identifying in silico a test ligand capable of interacting with a first conformation of the SARS-COV-2 Spike protein, a second conformation of the SARS-COV-2 Spike protein, or both to the first and the second conformation of the SARS-COV-2 Spike protein, wherein three-dimensional models of the first conformation and the second conformation of the SARS-COV-2 Spike protein are computationally derived and incorporate solvent accessibility information based on deuterium incorporation obtained HDX-MS analysis. The solvent accessibility information may include hydrogen exchange rates calculated based on the deuterium incorporation data obtained by the HDX-MS analysis. In some instances, hydrogen exchange rates may be used as constraints in three dimensional models of SARS-COV-2 Spike protein or its fragments created by other structural biology methods, such as, but not limited to, X-ray crystallography, SAXS, or cryoEM. The three-dimensional model used in computational docking may be a three-dimensional model of a monomer of a

SARS-COV-2 Spike protein, or a fragment of the monomer of a SARS-COV-2 Spike protein, which includes amino acid residues that are solvent-exposed in the second conformation but not in the first conformation of the of the SARS-COV-2 Spike protein. The three-dimensional model may be a three-dimensional a trimer of the SARS-COV-2 Spike protein or a fragment of the trimer of the SARS-COV-2 Spike protein comprising amino acid residues that are solvent-exposed in the second conformation but not in the first conformation of the of the SARS-COV-2 Spike protein. The above and other computational methods according to the embodiments of the present invention may use computational docking between a test ligand and SARS-COV-2 Spike protein. Computationally identified candidate ligands identified may be further tested by one more in vitro assays for their ability to bind to the SARS-COV-2 Spike protein.

### D. Molecular Modeling

Various terms and concepts are employed in computational modeling of molecules. Typically, a set of appropriate molecular descriptors describing each distinct configuration of include will be used to distinguish one configuration from another. Molecular descriptors may include, but are not limited to, a) chemical descriptors (e.g., element, atom type, chemical group, residue, bond type, hybridization state, ionization state, tautomeric state, chirality, stereochemistry, protonation, hydrogen bond donor or acceptor capacity, aromaticity, etc.); b) physical descriptors (e.g., charge, both formal and partial, mass, polarizability, ionization energy, characteristic size parameters, such as van der Waals [vdW] radii, vdW well depths, hydrophobicity, hydrogen bonding potential parameters, solubility, equilibrium bond parameters relating bond energies to bond geometries, etc.); c) geometrical descriptors (e.g., atomic coordinates, bond vectors, bond lengths, bond angles, bond torsions, suitable structural descriptors for rings, descriptors for molecular surfaces and volumes, such as solvent accessible surfaces and solvent-excluded volumes, etc.); and d) environmental descriptors (e.g., temperature, pH, ionic strength, pressure, etc.). Chemical descriptors may be assigned based on application of one or more rules or concepts of organic (or inorganic, if appropriate) chemistry to represent chemical structures that must at least stipulate basic structural information such as element type and bond connectivity (i.e., minimally which nonhydrogen atoms are connected to one another) but may also contain some form of coordinate information. Such chemical structures may be stored and received in a number of different data representations. One common example of data representation, though many others are also possible, is that of a pdb file. Examples of currently available software programs that can be used to assign chemical descriptors include SYBYL™ from Tripos, Chimera™ from UCSF, and WhatIf™ (for proteins), etc. Correct assignment of chemical descriptors may also include additional input regarding chiral centers and stereochemistry or even environmental factors, such as expected pH as related to assignment of ionization states.

“Binding mode” and the related terms and expression may refer to the 3-D molecular structure of a potential molecular complex in a bound state at or near a minimum of the binding energy (i.e., maximum of the binding affinity), where the term “binding energy” (sometimes interchanged with “binding free energy” or with its conceptually antipodal counterpart “binding affinity”) refers to the change in free energy of a molecular system upon formation of a potential molecular complex, i.e., the transition from an unbound to a (potential) bound state for the ligand and target. The term “system pose” is also sometimes used to refer to the binding mode. Here the term free energy generally refers to both enthalpic and entropic effects as the result of physical interactions between the constituent atoms and bonds of the molecules between themselves (i.e., both intermolecular and intramolecular interactions) and with their surrounding environment. Examples of the free energy are the Gibbs free energy encountered in the canonical or grand canonical ensembles of equilibrium statistical mechanics.

In general, the optimal binding free energy of a given target-ligand pair directly correlates to the likelihood of combination or formation of a potential molecular complex between the two molecules in chemical equilibrium, though, in truth, the binding free energy describes an ensemble of (putative) complexed structures and not one single binding mode. However, in computational modeling, it is usually assumed that the change in free energy is dominated by a single structure corresponding to a minimal energy. This is certainly true for tight binders (pK˜0.1 to 10 nanomolar) but questionable for weak ones (pK˜10 to 100 micromolar). The dominating structure is usually taken to be the binding mode. In some cases, it may be necessary to consider more than one alternative binding mode when the associated system states are nearly degenerate in terms of energy.

Binding affinity is of direct interest to drug discovery and rational drug design because the interaction of two molecules, such as a protein that is part of a biological process or pathway and a drug candidate sought for targeting a modification of the biological process or pathway, often helps indicate how well the drug candidate will serve its purpose. Furthermore, where the binding mode is determinable, the action of the drug on the target can be better understood. Such understanding may be useful when, for example, it is desirable to further modify one or more characteristics of the ligand so as to improve its potency (with respect to the target), binding specificity (with respect to other target biopolymers), or other chemical and metabolic properties.

When computationally modeling the nature and/or likelihood of a potential molecular combination for a given target-ligand pair, the actual computational prediction of binding mode and affinity is customarily accomplished in two parts: (a) “docking”, in which the computational system attempts to predict the optimal binding mode for the ligand and the target and (b) “scoring”, in which the computational system attempts to refine the estimate of the binding affinity associated with the computed binding mode. During library screening, scoring may also be used to predict a relative binding affinity for one ligand vs. another ligand with respect to the target molecule and thereby rank prioritize the ligands or assign a probability for binding. Scoring may include determining, for complexes of a particular target-pair, one or more of binding forces, configurational entropy, local minima in a Gibbs free energy landscape, or energy barriers between the local minima in the Gibbs free energy landscape. In this context, configurational entropy is the portion of a complex's entropy that is related to discrete representative positions of its constituent subparts. Gibbs free energy landscape is a representation (such as a graph) Gibbs free energy levels across different configurations of the complex. Scoring involves determining a docking score for a plurality of docked orientations of a three-dimensional model of one or more ligands relative to a three-dimensional model of a target. The docking score corresponds to a computational result for a particular computational program and energy function, and that can predict binding free energy and binding affinity, or to at least rank different complexes according to those parameters.

Docking may involve a search or function optimization algorithm, whether deterministic or stochastic in nature, with the intent to find one or more system poses that have favorable affinity. Scoring may involve a more refined estimation of an affinity function, where the affinity is represented in terms of a combination of one or more empirical, molecular-mechanics-based, quantum mechanics-based, or knowledge-based expressions, i.e., a scoring function. Individuals scoring functions may themselves be combined to form a more robust consensus-scoring scheme using a variety of formulations. In practice, there are many different docking strategies and scoring schemes employed in the context of today's computational drug design.

Whatever the choice of computational method there are inherent trade-offs between the computational complexity of both the underlying molecular models and the intrinsic numerical algorithms, and the amount of computing resources (time, number of CPUs, number of simulations) that must be allocated to process each molecular combination. For example, while highly sophisticated molecular dynamics simulations (MD) of the two molecules surrounded by explicit water molecules and evolved over trillions of time steps may lead to higher accuracy in modeling the potential molecular combination, the resultant computational cost (i.e., time and computing power) is so enormous that such simulations are intractable for use with more than just a few molecular combinations. On the other hand, the use of more primitive models for representing molecular interactions, in conjunction with multiple, and often error-prone, modeling shortcuts and approximations, may result in more acceptable computational cost, but will decrease modeling accuracy and predictive power.

Methods and concepts related to computational aspects of drug discovery and drug design are described in the publications summarized below. The process of high throughput docking and scoring and its applications are discussed in (43) and (44). A general approach to the design, docking, and virtual screening of multiple combinatorial libraries against a family of proteins is described in (45). The use of multiple computers to accelerate virtual screening of a large ligand library against a specific target by assigning groups of ligands to specific computers is described in (46). A number of examples of software tools are used to perform docking simulations. These methods involve a wide range of computational techniques, including use of a) rigid-body pattern-matching algorithms, either based on surface correlations, use of geometric hashing, pose clustering, or graph pattern-matching; b) fragmental-based methods, including incremental construction or ‘place and join’ operators; c) stochastic optimization methods including use of Monte Carlo, simulated annealing, or genetic (or memetic) algorithms; d) molecular dynamics simulations or e) hybrids strategies derived thereof. Computational docking may involve one or more of molecular dynamic simulations, kinetic Monte Carlo (KMC) simulations, direct simulations Monte Carlo (DSMC), or density functional theory (DFT) simulations to determine if a ligand binds to a particular target.

The earliest docking software tool was a graph-based rigid-body pattern-matching algorithm called DOCK, developed at UCSF back in 1982 (v1.0), with more recent versions including extensions to include incremental construction. Other examples of graph-based pattern-matching algorithms are described in include CLIX (which in turn uses GRID), FLOG and LIGIN. Other rigid-body pattern-matching docking software tools exist and include the shape-based correlation methods of FTDOCK and HEX, the geometric hashing and the pose clustering. In general, rigid-body pattern-matching algorithms assume that both the target and ligand are rigid (i.e., not flexible) and hence may be appropriate for docking small, rigid molecules (or molecular fragments) to a simple protein with a well-defined, nearly rigid active site. Thus, this class of docking tools may be suitable for de novo ligand design, combinatorial library design, or straightforward rigid-body screening of a molecule library containing multiple conformers per ligand. Incremental construction based docking software tools include FlexX from Tripos (licensed from EMBL), Hammerhead, DOCK v4.0 (as an option), and the nongreedy, backtracking algorithm. Programs using incremental construction in the context of de novo ligand design include LUDI (from Accelrys) and GrowMol. Docking software tools also include the tools based on ‘place and join’ strategies.

Incremental construction algorithms may be used to model docking of flexible ligands to a rigid target molecule with a well-characterized active site. They may be used when screening a library of flexible ligands against one or more targets. They are often comparatively less compute intensive, yet consequently less accurate, than many of their stochastic optimization based competitors. Incremental construction algorithms often employ one or more scoring functions to evaluate and rank different system poses encountered during computations. For example, FlexX was extended to FlexE to attempt to account for partial flexibility of the target molecule's active site via use of user-defined ensembles of certain active site rotamers.

Computational docking software tools based on stochastic methods include ICM (from MolSoft), GLIDE (from Schrodinger), and LigandFit (from Accelrys), all based on modified Monte Carlo techniques, as well as AutoDock v.2.5 (from Scripps Institute) based on simulated annealing. Other software tools are based on genetic or memetic algorithms and include GOLD, DARWIN, and AutoDock v.3.0 (also from Scripps).

Stochastic optimization-based methods may be used to model docking of flexible ligands to a target molecule. They generally use a molecular-mechanics-based formulation of the affinity function and employ various strategies to search for one or more favorable system energy minima. They are often more computer intensive, yet also more robust, than their incremental construction competitors. As they are stochastic in nature, different runs or simulations may often result in different predictions. Traditionally most docking software tools using stochastic optimization assume the target to be nearly rigid (i.e., hydrogen bond donor and acceptor groups in the active site may rotate), since otherwise the combinatorial complexity increases rapidly making the problem difficult to robustly solve in reasonable time.

Molecular dynamics simulations have also been used in the context of computational modeling of target-ligand combinations. In principle, molecular dynamics simulations may be able to model protein flexibility to an arbitrary degree. On the other hand, they may also require evaluation of many fine-grained, time steps and are thus often very time-consuming (one order of hours or even days per target-ligand combination). They also often require user interaction for selection of valid trajectories. Use of molecular dynamics simulations in lead discovery can be more suited to local minimization of predicted complexes featuring a small number of promising lead candidates. Hybrid methods may involve use of rigid-body pattern-matching techniques for fast screening of selected low-energy ligand conformations, followed by Monte Carlo torsional optimization of surviving poses, and finally even molecular dynamics refinement of a few choice ligand structures in combination with a (potentially) flexible protein active site.

There are a number of examples of scoring functions implemented in software and used to estimate target-ligand affinity, rank prioritize different ligands as per a library screen, or rank intermediate docking poses in order to predict binding modes. Scoring functions traditionally fall into three distinct categories: a) empirical scoring functions, b) molecular-mechanics-based expressions, or knowledge-based scoring functions or hybrid schemes derived thereof. Empirically derived scoring functions (as applied to target-ligand combinations) were first inspired by the linear free-energy relationships often utilized in QSAR studies. Empirical scoring functions include SCORE (used in FlexX), ChemScore, PLP, Fresno, and GlideScore v.2.0+(modified form of ChemScore, used by GLIDE).

In general, empirical scoring functions comprise the bulk of scoring functions used today, especially in the context of large compound library screening. The basic premise is to calibrate a linear combination of empirical energy models, each multiplied by an associated numerical weight and each representing one of a set of interaction components represented in a (so-called) ‘master scoring equation’, where said equation attempts to well approximate the binding free energy of a molecular combination. The numerical weight factors may be obtained by fitting to experimental binding free energy data composed for a training set of target-ligand complexes. Molecular-mechanics-based scoring functions were first developed for use in molecular modeling in the context of molecular mechanics force fields like AMBER, OPLS, MMFF, and CHARMM. Examples of molecular-mechanics-based scoring functions include both the chemical and energy-based scoring functions of DOCK v.4.0 (based on AMBER), the objective functions used in GOLD, AutoDock v.3.0 (with empirical weights), and FLOG. In general, molecular-mechanics-based scoring functions may closely resemble the objective functions utilized by many stochastic optimization-based docking programs. Such functions typically require atomic (or chemical group) level parameterization of various attributes (e.g., charge, mass, van der Waals radii, bond equilibrium constants, etc.) based on one or more molecular mechanics force fields (e.g., AMBER, MMFF, OPLS, etc.). In some cases, the relevant parameters for the ligand may also be assigned based on usage of other molecular modeling software packages, e.g., ligand partial charges assigned via use of MOPAC, AMPAC or AMSOL. They may also include intramolecular interactions (i.e., self-energy of molecules), as well as long range interactions such as electrostatics. In some cases, the combination of energy terms may again be accomplished via numerical weights optimized for reproduction of test ligand-target complexes.

Knowledge-based scoring functions were first inspired by the potential of mean

force statistical mechanics methods for modeling liquids. Examples include DrugScore, PMF and BLEEP. In general, knowledge-based scoring functions do not require partitioning of the affinity function. However, they do require usage of a large database of 3-D structures of relevant molecular complexes. There is also usually no need for regression against a data set of molecular complexes with known experimental binding affinities. These methods are based on the underlying assumption that the more favorable an interaction is between two atoms, at a given distance, the more frequent its occurrence relative to expectations in a bulk, disordered medium. These schemes are sometimes referred to as ‘inverse Boltzmann’ schemes, but in fact the presence of local, optimized structures in macromolecules and protein folds means that distance-dependent pair-wise preference distributions need not be strictly Boltzmann. It is also possible to introduce the concept of singlet preferences based on other molecular descriptors, e.g., solvent accessible surface area for approximation of solvation effects. Hybrid scoring functions may be a mixture of one or more scoring functions of distinct type. One example is VALIDATE, which is a molecular-mechanics/empirical hybrid function. Other combinations of scoring functions may include the concept of consensus scoring in which multiple functions may be evaluated for each molecular combination and some form of ‘consensus’ decision is made based on a set of rules or statistical criteria, e.g., states that occur in the top 10% rank list of each scoring function (intersection-based), states that have a high mean rank (average-based), etc. A useful review discussion of consensus scoring can be found in. Various file formats exist for the digital representation of structural and chemical information for both target proteins and compounds as related to structural databases. Examples include the pdb, mol2 (from Tripos), and the SMILES formats.

Computational solutions of electrostatic potentials in the classical regime range from simpler formulations, like those involving distance-dependent dielectric functions, to more complex formulations, like those involving solution of the Poisson-Boltzmann equation, a second order, generally nonlinear, elliptic partial differential equation. Other classical formalisms that attempt to model electrostatic desolvation include those based on the Generalized Born solvation model, methods that involve representation of reaction field effects via additional solvent accessible or fragmental volume terms, or explicit representation of solvent in the context of molecular dynamics simulations.

An exemplary modeling system for the analysis of molecular combinations according to embodiments of the present disclosure may operate as follows. A configuration modeler receives one or more input configuration records, including both the identities of and molecular descriptors for input structures for one or more molecular subsets from an input molecular combination database. The configuration modeler comprises a configuration data transformation engine, an affinity calculator, and descriptor data storage. Results from the configuration modeler are output as configuration results records to a results database (DB). Modeling system may be used to determine or characterize one or more molecular combinations. In some embodiments, this may include, but is not limited to, prediction of likelihood of formation of a potential molecular complex, or a proxy thereof, the estimation of the binding affinity or binding energy between molecular subsets in an environment, the prediction of the binding mode (or even additional alternative modes) for the molecular combination, or the rank prioritization of a collection of molecular subsets (e.g., ligands) based on predicted bioactivity with a target molecular subset, and would therefore also include usage associated with computational target-ligand docking and scoring.

In a typical operation, many molecular combinations, each featuring many different molecular configurations, may be modeled. Since the total possible number of configurations may be enormous, the modeling system may sample a subset of configurations during the modeling procedure, though the sampling subset may still be very large (e.g., millions or billions of configurations per combination) and the selection strategy for configuration sampling is specified by one or more search and/or optimization techniques (e.g., steepest descent, conjugate gradient, modified Newton's methods, Monte Carlo, simulated annealing, genetic or memetic algorithms, brute force sampling, pattern matching, incremental construction, fragment place-and-join, etc.). An affinity function is evaluated for each visited configuration and the results for one or more configurations recorded in a storage medium.

The molecular combination may then be assessed by examination of the set of configuration results including the corresponding computed affinity function values. Once the cycle of computation is complete for one molecular combination, modeling of the next molecular combination may ensue. Alternatively, in some embodiments of the modeling system, multiple molecular combinations may be modeled in parallel as opposed to in sequence. Likewise, in some embodiments, during modeling of a molecular combination, more than one configuration may be processed in parallel as opposed to in sequence.

In one embodiment, modeling system may be implemented on a dedicated microprocessor, ASIC, or FPGA. In another embodiment, modeling system may be implemented on an electronic or system board featuring multiple microprocessors, ASICs, or FPGAs. In yet another embodiment, modeling system may be implemented on or across multiple boards housed in one or more electronic devices. In yet another embodiment, modeling system may be implemented across multiple devices containing one or more microprocessors, ASICs, or FPGAs on one or more electronic boards and the devices connected across a network.

In some embodiments, modeling system may also include one or more storage media devices for the storage of various, required data elements used in or produced by the analysis. Alternatively, in some other embodiments, some or all of the storage media devices may be externally located but networked or otherwise connected to the modeling system. Examples of external storage media devices may include one or more database servers or file systems. In some embodiments involving implementations featuring one or more boards, the modeling system may also include one or more software processing components in order to assist the computational process. Alternatively, in some other embodiments, some or all of the software processing components may be externally located but networked or otherwise connected to the modeling system.

In some embodiments, results records from database may be further subjected to a configuration selector during which one or more molecular configurations may be selected based on various selection criteria and then resubmitted to the configuration modeler (possibly under different operational conditions) for further scrutiny (i.e., a feedback cycle). In such embodiments, the molecular configurations are transmitted as inputs to the configuration modeler in the form of selected configuration records. In another embodiment, the configuration selector may also send instructions to the configuration data transformation engine on how to construct one or more new configurations to be subsequently modeled by configuration modeler. For example, if the configuration modeler modeled ten target-ligand configurations for a given target-ligand pair, and two of the configurations had substantially higher estimated affinity than the other eight, then the configuration selector may generate instructions for the configuration data transformation engine on how to construct further additional configurations (i.e., both target and ligand poses) that are structurally similar to the top two high-scoring configurations, which are then subsequently processed by the remainder of the configuration modeler. In some embodiments, the transmitted instructions may relate to construction from the resubmitted configurations whereas in other cases they relate to construction from the original input reference configuration(s).

In some embodiments, once analysis of a molecular combination is completed (i.e., all desired configurations assessed) a combination postprocessor may be used to select one or more configuration results records from database in order to generate one or more qualitative or quantitative measures for the combination, such as a combination score, a combination summary, a combination grade, etc., and the resultant combination measures are then stored in a combination results database. In one embodiment, the combination measure may reflect the configuration record stored in database with the best observed affinity. In another embodiment, multiple high affinity configurations are submitted to the combination postprocessor and a set of combination measures written to the combination results database. In another embodiment, the selection of multiple configurations for use by the combination postprocessor may involve one or more thresholds or other decision-based criteria.

In a further embodiment, the selected configurations are also chosen based on criteria involving structural diversity or, alternatively, structural similarity (e.g., consideration of mutual rmsd of configurations, use of structure-based clustering or niching strategies, etc.). In yet another embodiment, the combination measures output to the combination results database are based on various statistical analysis of a sampling of possibly a large number of configuration results records stored in database. In other embodiment the selection sampling itself may be based on statistical methods (e.g., principal component analysis, multidimensional clustering, multivariate regression, etc.) or on pattern-matching methods (e.g., neural networks, support vector machines, etc.)

In yet another embodiment, the combination results records stored in database may not only include the relevant combination measures, but may also include some or all of the various configuration records selected by the combination postprocessor in order to construct a given combination measure. For example, combination results records stored in database may include representations of the predicted binding mode or of other alternative, high affinity (possibly structurally diverse) modes for the molecular combination. In another embodiment, the combination postprocessor may be applied dynamically (i.e., on-the-fly) to the configuration results database in conjunction with the analysis of the molecular combination as configuration results records become available. In yet another embodiment, the combination postprocessor may be used to rank different configurations in order to store a sorted list of either all or a subset of the configurations stored in database that are associated with the combination in question. In yet other embodiments, once the final combination results records, reflecting the complete analysis of the molecular combination by the configuration modeler, have been stored in database, some or all of the configuration records in database may be removed or deleted in order to conserve storage in the context of a library screen involving possibly many different molecular combinations. Alternatively, some form of garbage collection or equivalent may be used in other embodiments to dynamically remove poor affinity configuration records from database .

In one embodiment, the molecular combination record database may comprise one or more molecule records databases (e.g., flat file, relational, object oriented, etc.) or file systems and the configuration modeler receives an input molecule record corresponding to an input structure for each molecular subset of the combination, and possibly a set of environmental descriptors for an associated environment. In another embodiment, when modeling target protein-ligand molecular combinations, the molecular combination record database is replaced by an input target record database and an input ligand (or drug candidate) record database. In a further embodiment, the input target molecular records may be based on that are experimentally derived (e.g., X-ray crystallography, NMR, etc.), energy minimized, and/or model-built structures. In another embodiment, the input ligand molecular records may reflect energy minimized or randomized 3-D structures or other 3-D structures converted from a 2-D chemical representation, or even a sampling of low energy conformers of the ligand in isolation. In yet another embodiment, the input ligand molecular records may correspond to naturally existing compounds or even to virtually generated compounds, which may or may not be synthesizable.

In one embodiment the configuration data transformation engine may transform one or more input molecular configurations into one or more other new configurations by application of various geometrical operators characterized by sets of geometrical descriptors. Transformation of molecular configurations into newer variants may be accomplished by one or more unary operations (i.e., acting on one input configuration, such as the mutation operator in a genetic algorithm), binary operations (i.e., acting on two input configurations, such as a binary crossover in a genetic algorithm), other n-ary operations (i.e., acting on a plurality of input configurations, such as a transform operator based on a population of configurations), or a combination thereof. In another embodiment, the transformation of molecular configurations into newer variants may result in multiple new configurations from one configuration, such as, for example, the construction of a suitable (often randomized) initial population for use in a genetic algorithm. In some embodiments, the configuration data transformation engine may be able to construct ab initio one or more entirely new configurations without the requirement of input geometrical descriptors from an input molecular combination database, though other types of molecular descriptors may still be needed.

As already discussed, in some embodiments, the set of configurations generated via transformation during the course of an analysis of a molecular combination may be determined according to a schedule or sampling scheme specified by one or more search and/or optimization techniques used to drive the modeling processes of the configuration modeler. In some embodiments, the search strategy or optimization technique may be an iterative process whereby one or more configurations are generated from one or more input configurations, then affinities are calculated for each configuration, decisions are made based on affinity and/or structure, and all or part of the new set of configurations are used as input seeds for the next iteration; the process continuing until a specified number of iterations are completed configuration modeler 102 or some other convergence criteria satisfied. In such embodiments, the input configuration records 106 obtained or derived from data in the input molecular combination database, may serve only to initiate (or also possibly reset) the iterative process (i.e., prime the pump).

In some embodiments, the search strategy or optimization technique may be stochastic in nature meaning that the set of configurations visited during analysis of a molecular combination may involve some random component and thus be possibly different between different runs of the configuration modeler as applied to the same molecular combination. Here the term run refers to two different initiations of (possibly iterative) cycles of computation for analysis of the same molecular combination. In some embodiments, the combination postprocessor may then base its results or decisions on configuration results records stored in database but obtained from different runs. In some embodiments, the configuration data transformation engine may produce new configurations sequentially, such as a new possible state associated with a given iteration of a Monte Carlo-based technique, and feed them to the affinity calculator in a sequential manner. In other embodiments, the configuration data transformation engine may produce multiple new configurations in parallel, such as a population associated with a given iteration of a genetic algorithm, and submit them in parallel to the affinity calculator. In other embodiments, the configuration data transformation engine may not generate additional configurations and instead the configuration modeler may operate solely on one or more input configuration records from the input molecular combination database, such as for example in some usages of modeling system related to scoring of a set of known molecular configurations. In such embodiments, the configuration data modeler may not include a search or optimization strategy and instead be used to perform affinity calculations on an enumerated set of input configuration records.

In some embodiments, various descriptor data related to the configurations of a given molecular combination may be stored or cached in one or more components of a descriptor data storage via one or more storage (or memory) allocation means, structure or apparatus for efficient access and storage during the cycle of computations performed by the configuration modeler. In one embodiment, the descriptor data storage may contain chemical or physical descriptors assigned to atoms, bonds, groups, residues, etc. in each of the molecular subsets or may even also contain environmental descriptors. In another embodiment, the descriptor data common to all configurations for a given molecular combination is compactly represented via a storage allocation means in one or more lookup tables. For example, often many physical and chemical descriptors may be identical for different configurations of a combination whereas one or more geometric descriptors are not. In yet another embodiment, the descriptor data storage may also contain relevant geometric descriptors for the configurations arranged in one or more storage formats via a prescribed storage allocation means. As examples, such formats may involve, but are not limited to, records analogous to pdb or mol2 file formats. Additional examples include various data structures such as those associated with the molecular representation partitioning shown in Ahuja I. As a further example, perhaps stored descriptors for atoms and bonds may represent individual nodes in one or more lists or arrays, or may alternatively be attached, respectively, to nodes and edges of a tree or directed graph.

The whole or parts of the input configuration records, and, if applicable, selected configuration records chosen by configuration selector, may be converted to data representations used in the storage allocation means of the descriptor data storage. Data constructs contained in the descriptor data storage may be either read (i.e., accessed) for use by the configuration data transformation engine or the affinity calculator and may be written either at the inception of or during the execution of a cycle of computation by the configuration modeler. The layout and access patterns for the associated descriptor data storage will likely depend on the needs of the affinity calculator as well as the configuration data transformation engine.

The affinity calculator may comprise one or more processing (i.e., affinity) engines, where each affinity engine may be dedicated to performing calculations related to one or more affinity components as defined previously in regard to interaction types, affinity formulations, and computation strategies. In some embodiments, different affinity engines are assigned to each unique affinity component. In other embodiments, one or more affinity engines may compute multiple affinity components according to similarity of processing requirements. In yet other embodiments, different affinity engines may be grouped or otherwise arranged together to take advantage of common subsets of required input data in order to improve any caching scheme and/or to reduce the number of, the bandwidth requirements for, or the routing requirements for various associated data paths.

For example, in one embodiment, affinity components for both the electrostatic and van der Waals interactions involving field-based computation strategies utilizing stored pregenerated probe grid maps, may be computed on the same affinity engine, where said engine requires access to both types of probe grid maps in storage and to various numerical parameters used in evaluating the affinity formulation for the two different interactions. As another example, affinity components for both the hydrogen bonding and van der Waals interactions using affinity formulations featuring generalized Lennard-Jones potentials computed according to a pair-based computation strategy may be computed on the same affinity engine. In an alternative embodiment, the same two affinity components may be computed using two different affinity engines but grouped together in order to share common input data such as that relating to spatial coordinates and a subset of relevant chemical or physical descriptors.

### V. COMPUTER SYSTEMS

Any of the computer systems mentioned in the present disclosure may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. The subsystems can be interconnected via a system bus. Additional subsystems such as a printer, keyboard, storage device(s), monitor, which is coupled to display adapter, and others are shown. Peripherals and input/output (I/O) devices, which couple to I/O controller, can be connected to the computer system by any number of means known in the art, such as serial port. For example, serial port or external interface (e.g. Ethernet, Wi-Fi, etc.) can be used to connect computer system to a wide area network such as the Internet, a mouse input device, or a scanner. The interconnection via system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g. an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### VI. IN VITRO ANALYTICAL METHODS

The methods described in the present disclosure can include determining binding properties and/or biological activity (including presence, absence or amount of biological activity, which can be also referred of “efficacy,” of a ligand in an in vitro biological assay or in vivo in a subject (such as a model animal, for example, a wild-type animal, a laboratory-bred animal, or a transgenic animal model). Some of the methods described in the present disclosure can include validating or confirming in silico predicted activities of a ligand, for example, in silico binding of the ligand to the target protein, with the results of an in vitro biological assay, and/or with the results of an in vivo study in an animal model. One exemplary assay in vitro suitable for evaluation of the the ability of candidate

ligands to bind to SARS-COV-2 Spike protein is an enzyme-linked immunosorbent assays (ELISA). For instance, a ligand can be coated on suitable ELISA plates, which are subsequently washed, blocked, and incubated under suitable conditions with an aquesous solution (such as serial dilutions) of SARS-COV-2 Spike protein. After incubation, the plates are washed and exposed to an anti-SARS-COV-2 Spike protein antibody that is linked directly, or via a secondary antibody, to a reporter enzyme. After subsequent washing, the activity of the reporter enzyme (and, hence, the bound SARS-COV-2 Spike protein) is detected by incubating the plates with an appropriate substrate to produce a measurable product. Another exemplary assay is bio-layer interferometry (BLI). BLI can be performed, for example, on the Octet® system (Sartorius, Göttingen, Germany). Some other exemples of suitable in vitro assays are surface-plasmon resonance (SPR) gel-shift assays, fluorescence polarization assay, fluorescence anisotropy assay, and isothermal calorimetry (ITC).

### EXAMPLES

The following examples are offered to illustrate, but not to limit the claimed invention.

### VII. EXAMPLE 1: METHODS

### A. Protein Expression and Purification.

SARS-COV-2 Spike (2P) and RBD were expressed and purified from stably transformed Expi293 cells, following the methods substantially as described in (39). HexaPro, HexaPro S383C/D985, and UK HexaPro were expressed and purified from transiently transfected ExpiCHO cells substantially as described in (30). ACE2-Fc is described in (40). 3A3 IgG (“3A3 antibody”) was expressed and purified from ExpiCHO cells substantially as described in (27).

### B. Continuous Hydrogen Exchange Labeling.

For all continuous hydrogen exchange experiments, deuterated buffer was prepared by lyophilizing PBS (pH 7.4, Sigma-Aldrich P4417) and resuspending in D₂O (Sigma-Aldrich 151882). To initiate the continuous labeling experiment, samples were diluted 10-fold (final spike trimer concentration of 0.167 μM) into temperature equilibrated deuterated PBS buffer (pH_{read} 7, pD 7.4). Samples were quenched, at the time points outlined below, by mixing 30 μL of the partially exchanged protein with 30 μL of 2× quench buffer (3.6 M GdmCl, 500 mM TCEP, 200 mM Glycine pH 2.4) on ice. Samples were incubated on ice for 1 minute to allow for partial unfolding to assist with proteolytic degradation and then flash frozen in liquid nitrogen and stored at −80° C.

For studies comparing HexaPro +ACE2 and the RBD in isolation vs in S-2P, purified SARS-COV-2 Spike protein (1.67 μM SARS-COV-2 Spike protein trimer or 5 μM RBD) was incubated in PBS at 25° C. overnight (12-16 hours) before the initiation of hydrogen exchange. For experiments done in the presence of ACE2-Fc, the ligand was added during this incubation at a 1.25:1 molar ratio of ligand to SARS-COV-2 Spike protein monomer (6.25 μM ligand) to ensure saturation. Based on the reported affinity (K_{D}˜15 nM) for ACE2-Fc, fraction bound was assumed to be greater than 97%. The hydrogen exchange time points for these experiments were 15 seconds, 60 seconds, 180 seconds, 600 seconds, 1800 seconds, 5400 seconds, and 14400 seconds.

For the comparison of HexaPro±, HexaPro was incubated overnight at 37° C. (12-16 hours). After incubation the protein was moved to 25° C. and diluted to 1.67 μM spike trimer. In the 3A3 bound condition, 6.25 μM antibody was added and allowed to bind for 10 minutes at 25° C. Given the affinity of 3A3 antibody for HexaPro (12 nM, fraction bound can be assumed to be greater than 97%. The quench time points for this experiment were 15 seconds, 180 seconds, 1800 seconds and 14400 seconds.

### C. Back Exchange Control Preparation.

S-2P was diluted to 1.67 μM trimer in PBS pH 7.4. To initiate hydrogen exchange,

the sample was diluted 10-fold (final trimer concentration of 0.167 μM) into deuterated PBS buffer (pH_{read} 7, pD 7.4) that was supplemented with 3.6 M GdmCl, and then incubated at 37° C. The addition of denaturant and increased temperature both promote hydrogen exchange by destabilizing folded structures and increasing the intrinsic rate of hydrogen exchange, respectively. Following two weeks of exchange, 30 μL of deuterated protein was mixed with 30 μL of 2× quench buffer lacking denaturant (500 mM TCEP, 200 mM Glycine pH 2.4) and kept on ice for one minute prior to flash freezing in liquid nitrogen and storage at −80° C. The results of this control experiment were used to characterize the back exchange of the system and were not used to adjust deuteration values of continuous-labeling experiments.

### D. Incubation Kinetics and Pulse Labeling.

For evaluating the temperature dependent kinetics of interconversion, frozen protein samples were thawed, diluted to 5 μM SARS-COV-2 Spike protein monomer, and incubated at 37° C. overnight. The samples were then moved to a temperature-controlled chamber at 4° C. and the population of each state was evaluated at the specified time points as described below. After the final 4° C. sample was taken (96-526 hours, depending on the construct), the sample was returned to a 37° heat block for further incubation, and the population of each state was again evaluated at the specified time points, as described below. To evaluate the relative population of the A and B state at each time point, 3 μL of the sample was removed from the incubation tube and mixed with 27 μL of room temperature deuterated buffer. After a 1-minute labeling pulse, 30 μL of quench buffer kept on ice was mixed with the 30 μL of labeled protein. Quenched samples were kept on ice for 1 minute to allow for partial unfolding, and then flash frozen in liquid nitrogen.

For kinetics carried out in the presence of ACE2 or 3A3 antibody, after the initial 37° C. incubation, the sample was brought to 25° C. and ligand was added (6.25 μM). To monitor the population of state A and B as a function of time at 25° C. in the presence of ACE2, the sample was kept in a temperature-controlled chamber at 25° C., and aliquots were removed for pulse labeling, as described above, at 0 hours, 30 minutes, 3 hours, 6 hours and 24 hours.

### E. Protease Digestion and LC MS.

All samples were thawed immediately before injection into a cooled valve system (Trajan LEAP) coupled to a LC (Thermo UltiMate 3000). Sample time points were injected in random order. The temperature of the valve chamber, trap column, and analytical column were maintained at 2° C. The temperature of the protease column was maintained at 10° C. The quenched sample was subjected to inline digestion by two immobilized acid proteases, aspergillopepsin (Sigma-Aldrich P2143) and porcine pepsin (Sigma-Aldrich P6887) (in that order), at a flow rate of 200 μL/min of buffer A (0.1% formic acid). Protease columns were prepared in house by coupling protease to beads (Thermo Scientific POROS 20 Al aldehyde activated resin 1602906) and packed by hand into a column (2 mm ID×2 cm−IDEX C-130B). Following digestion, peptides were desalted for 4 minutes on a hand-packed trap column (Thermo Scientific POROS R2 reversed-phase resin 1112906, 1 mm ID×2 cm−IDEX C-128). Peptides were then separated with a C8 analytical column (Thermo Scientific BioBasic-8 5 μm particle size 0.5 mm ID×50 mm 72205-050565) and a gradient of 5-40% buffer B (100% Acetonitrile, 0.1% Formic Acid) at a flow rate of 40 μL/min over 14 minutes, and then of 40-90% buffer B over 30 seconds. The analytical and trap columns were then subjected to a sawtooth wash and equilibrated at 5% buffer B prior to the next injection. Protease columns were washed with two injections of 100 μL 1.6 M GdmCl, 0.1% formic acid prior to the next injection. Peptides were eluted directly into a Q Exactive Orbitrap Mass Spectrometer operating in positive mode (resolution 70000, AGC target 3e6, maximum IT 50 ms, scan range 300-1500 m/z). For each SARS-COV-2 Spike protein construct, a tandem mass spectrometry experiment was performed (Full MS settings the same as above, dd-MS2 settings as follows: resolution 17500, AGC target 2e5, maximum IT 100 ms, loop count 10, isolation window 2.0 m/z, NCE 28, charge state 1 and ≥7 excluded, dynamic exclusion of 15 seconds) on undeuterated samples.

### F. Peptide Identification.

Byonic (Protein Metrics) was used to identify unmodified and glycosylated peptides in the tandem mass spectrometry data. The sequence of the expressed construct, including signal sequence and trimerization domain, was used as the search library. Sample digestion parameters were set to non-specific. Precursor mass tolerance and fragment mass tolerance was set to 6 and 10 ppm, respectively. Variable N-linked glycosylation was allowed, with a library of 132 human N-glycans used in the search. No non-glycosylated peptides spanning any of the 22 known glycosylation sites in SARS-COV-2 Spike sequence were ever observed, independent of the glycosylation search parameters. Peptide lists (sequence, charge state, and retention time) were exported from Byonic and imported into HDExaminer 3 (Sierra Analytics). When multiple peptide lists were obtained, all were imported and combined in HDExaminer 3.

### G. HDExaminer 3 Analysis.

Peptide isotope distributions at each exchange time point were fit in HDExaminer 3. For glycosylated peptides, only the highest confidence modification was included in the mass spectra search and analysis. For unimodal peptides, deuteration levels were determined by subtracting mass centroids of deuterated peptides from undeuterated peptides. For bimodal peaks, extracted peptide isotope spectra were exported from HDExaminer 3 and analyzed separately (see below for details)

### H. Bimodal Fitting and Conformation Quantification.

Peptide mass spectra for bimodal peptides were exported from HDExaminer 3.0. All quantitative analysis of the exported peptide mass spectra was performed using python scripts in Jupyter notebooks. After importing a peptide mass spectra, the m/z range containing all possible deuteration states of the selected peptide was isolated and the find peaks method from the SciPy.signal package, available, for example, from docs.scipy.org/doc/scipy/reference/signal.html. was used to identify each isotope in the mass envelope and the height of each peak was used as its intensity. The area of the total mass envelope was normalized to account for run-to-run differences in intensity.

The bimodal mass envelopes for all time points for the same version of SARS COV 2 Spike protein tested under the same conditions were globally fit to a sum of two Gaussians, keeping the center and width of each Gaussian constant across all incubation time points.

Global fitting here refers to fitting a parameter to a particular Gaussian distribution, while the parameter to be the same for all data sets. For example, instead of identifying the best Gaussian center and width for a particular distribution, Gaussian center and width are identified that best describe all the distributions. Fitting was done using the curve_fit function from the SciPy.optimize package (available, for example, from docs.scipy.org/doc/scipy/reference/optimize.html). After fitting, the area under each individual Gaussian was determined to approximate the relative population of each state.

### VIII. EXAMPLE 2: HDX-MS ON S-2P

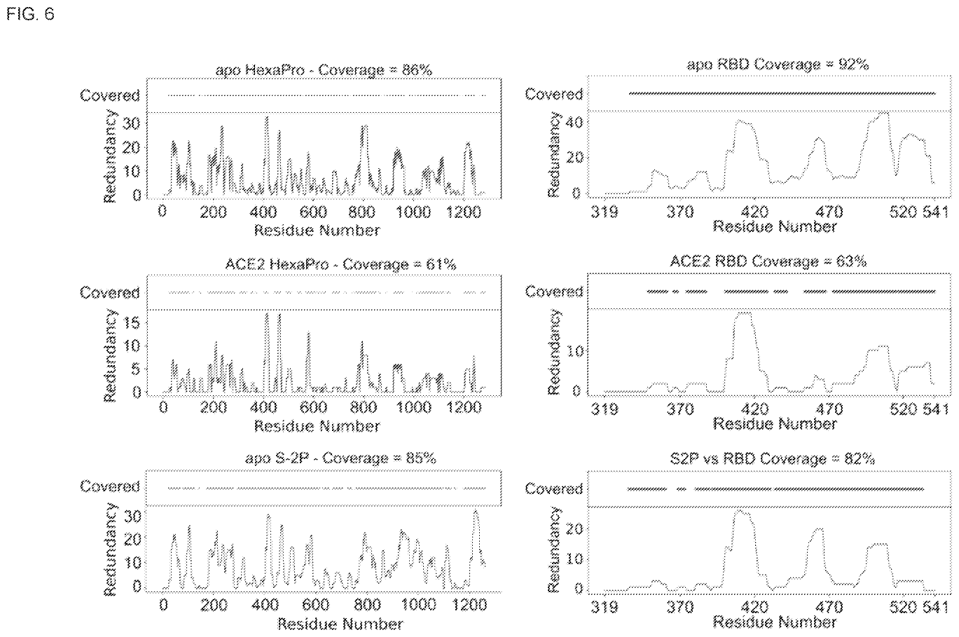

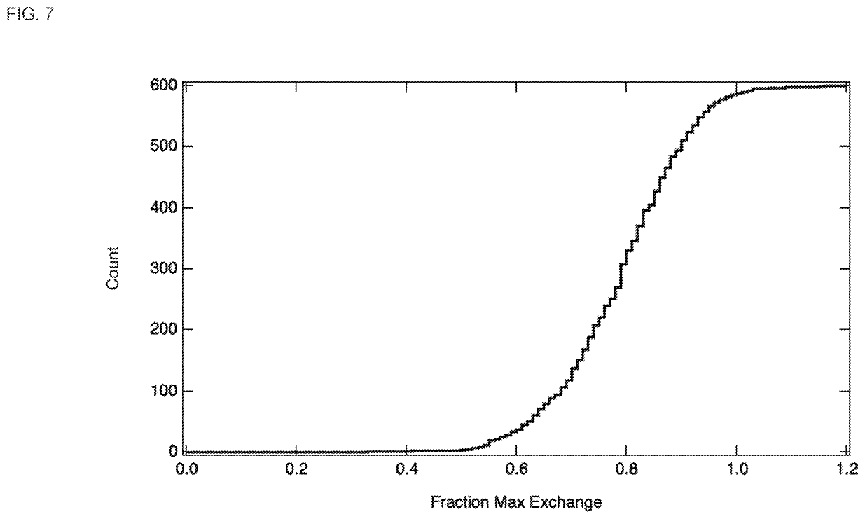

The inventors first followed the time course of hydrogen exchange on the entire S-2P ectodomain, over a period of 15 seconds to 4 hours (see Example 1). Using a combination of porcine pepsin and aspergillopepsin digestion, the inventors obtained 85% peptide coverage, allowing for interrogation the dynamics of the entire protein (800 peptides, which include 8 of the 22 glycosylation sites, average redundancy of 8.6) (FIG. 6). Notably, peptide coverage was provided coverage in areas not resolved in the cryoEM structure, including loops in the N-terminal domain (NTD) and RBD that have been found to be recognized by antibodies, loops in the S2 region that include the protease cleavage sites, and C-terminal amino acid residues after amino acid residue 1145 which includes the second heptad repeat (HR2). Based on control experiments using deuterated protein, the HDX protocol resulted in an average back exchange of 22%.

The vast majority of peptides showed a classic single isotopic envelope whose centroid increases in mass as deuterons were added over time. A fraction of the peptides, however, showed bimodal behavior—with two isotopic envelopes both increasing in mass over time as deuterons are added over time of incubation with D₂O: one less-exchanged distribution (with a centroid at lower m/z), and a second more-exchanged distribution, with a centroid at higher m/z, As illustrated in FIG. 1B, for a peptide exhibiting bimodal behavior, a unimodal m/z distribution is observed at the start of the incubation with D₂O, due to the fact that no deuterons were yet added. Eventually, at least theoretically, full deuterium exchange may occur in both conformation of a particular peptide, leading to a unimodal m/z distribution.

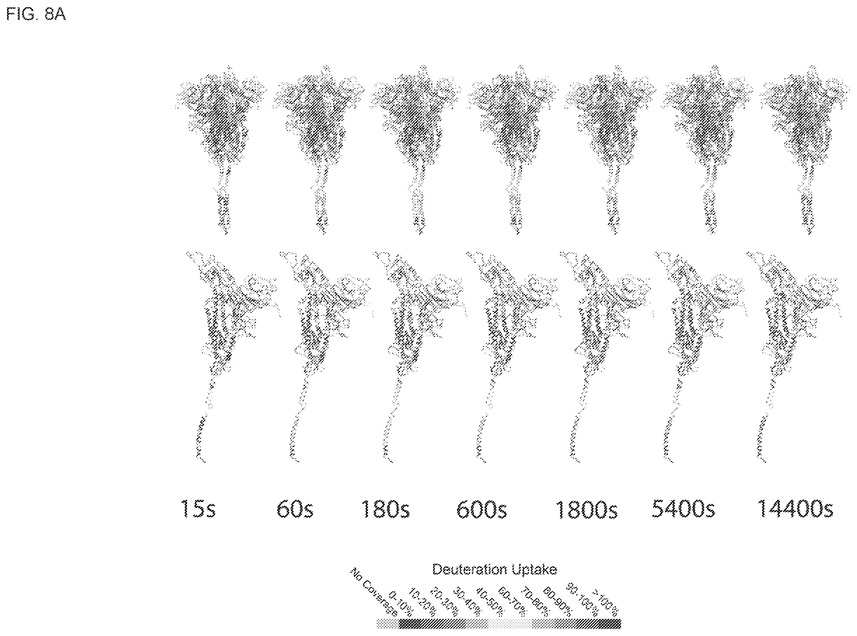

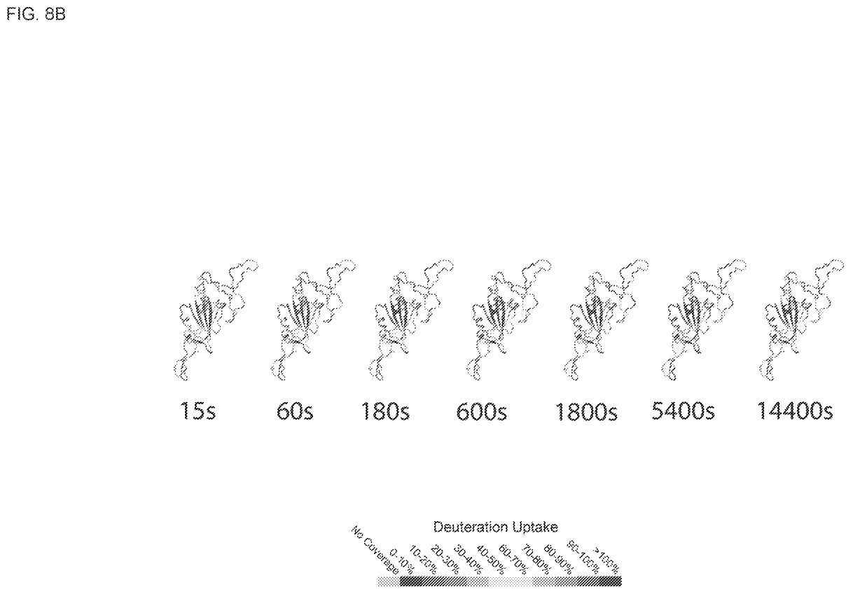

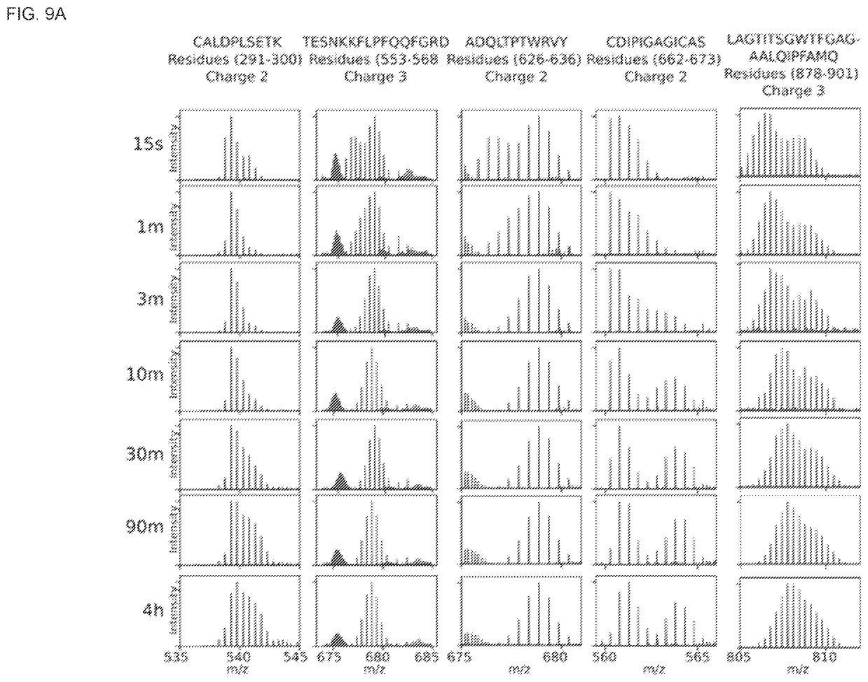

The peptides that exhibited bimodal behavior (“bimodal peptides”) in the HDX-MS experiments are described in detail below. The HDX profile of all the peptides, with the exception of the more-exchanged distributions in the bimodal peptides, was consistent with the known pre-fusion conformation (FIG. 1C; FIG. 8A; FIG. 8B): secondary structure and buried elements within the trimer exchange slower than exposed loops. The inventors also observed protection for amino acid residues 1140-1197, which includes HR2, a region not defined in single-particle cryoEM structures, supporting the predicted helical structure of this region (24) and the relative rigidity of the stalk observed by cryo-electron tomography (cryo-ET) (25).

### IX. EXAMPLE 3: IDENTIFICATION OF AN ALTERNATIVE CONFORMATION

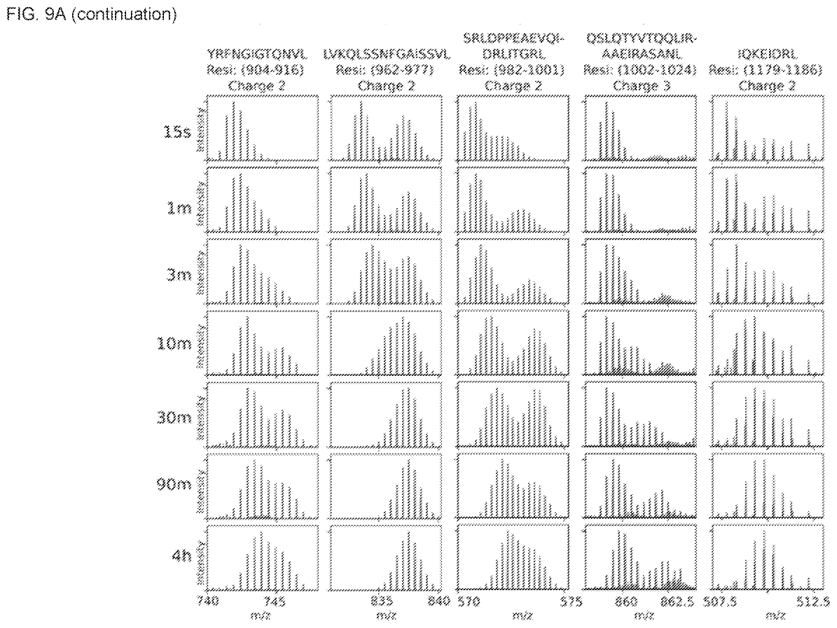

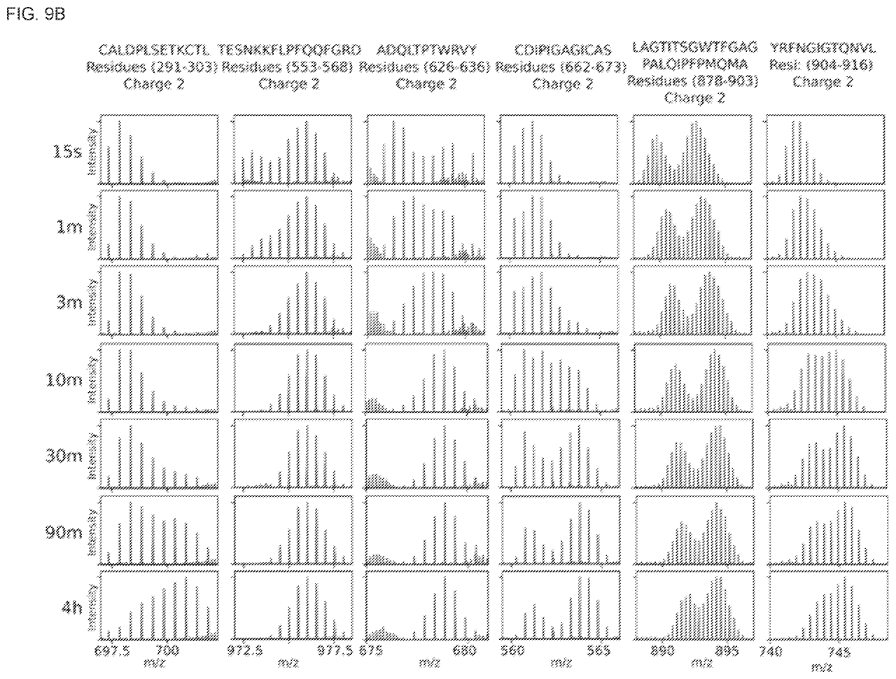

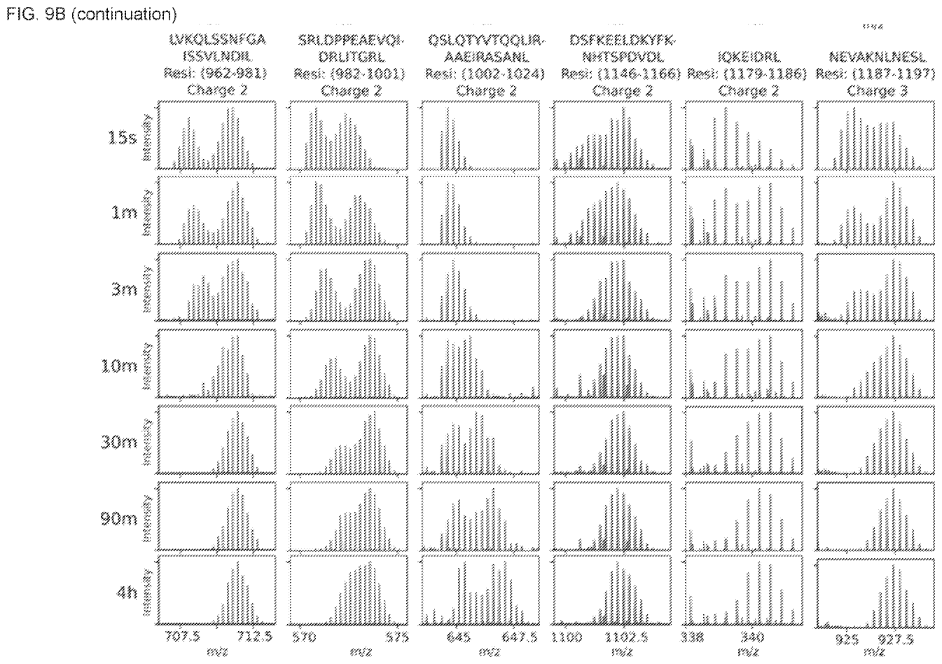

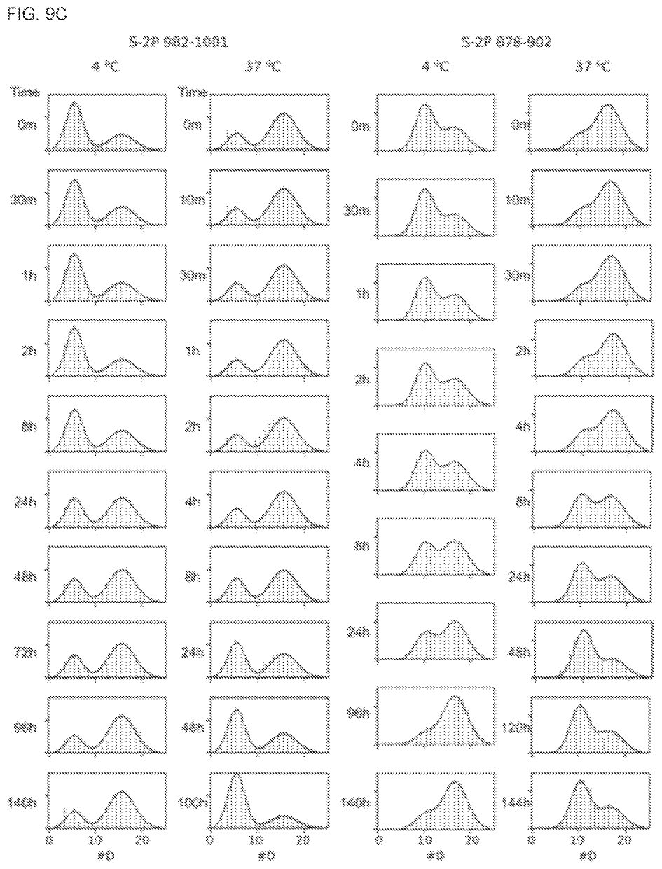

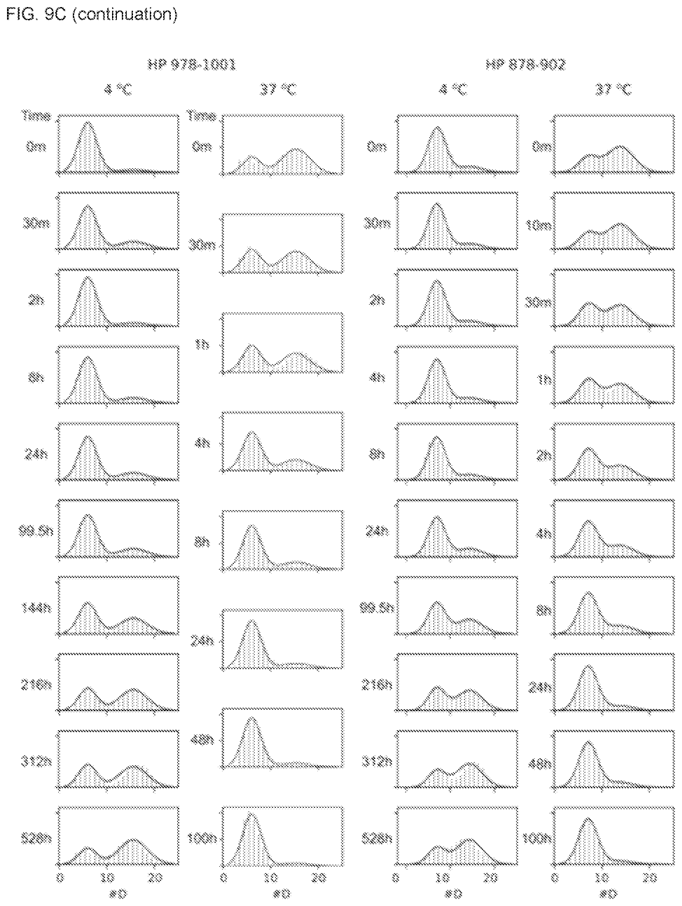

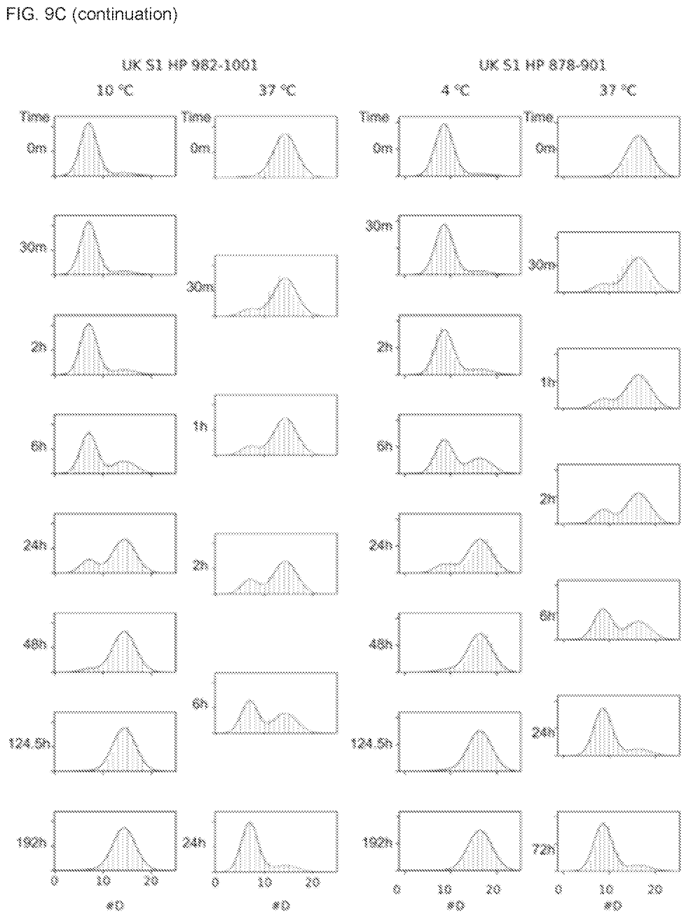

Bimodal mass envelopes can indicate the presence of two different conformations that interconvert slowly on the timescale of the hydrogen exchange experiment: one where the amides are more accessible to exchange compared to the other. However, it can also be a result of the kinetics of the hydrogen exchange process itself, so-called EX1 exchange (when the rates of hydrogen bond closing are much slower than the intrinsic chemistry of the exchange process). In this rare scenario, the heavier mass distribution will increase in intensity at the expense of the lighter one over the observed time period. This is not what the inventors observed for the SARS-COV-2 Spike protein: the bimodal mass distributions retained their relative intensities, increasing in average mass over time (FIGS. 9A, 9B and 9C). The relative population of each state was the same for every bimodal peptide under any given condition. Thus, these bimodal peptides reflected two different conformations; they reported on the regions of the protein that showed differences in hydrogen exchange in each conformation.

The bimodal peptides the inventors observed were predominantly in the most conserved region of SARS-COV-2 Spike protein—the S2 region (26) (FIG. 2A). When mapped onto the canonical pre-fusion conformation, many of the bimodal peptides were mapped to the helices at the trimer interface (regions that include amino acid residues 962-1024, 1146-1166, 1187-1196), suggesting that these helices are either less stable or more solvent exposed in the newly identified conformation. The inventors also observed bimodal peptides in other regions of the inter-protomer (trimeric) interface, such as the region including amino acid residues 870-916 in S2, region including amino acid residues 553-574 in S1, and region including amino acid residues 662-673 in S1, again suggesting a loss of trimer contacts in these regions. Finally, the inventors observed bimodal peptides in two regions that do not form interprotomer contacts (such as a region containing amino acid residues 291-305, and a region containing amino acid residues 626-636); instead, these regions form the interface between the NTD and second SI subdomain (SD2), suggesting that this subdomain interface is also lost in the newly identified second conformation. Besides the bimodal peptides, all the other peptides fit to a classic unimodal distribution in the mass spectrum, suggesting they behave similarly in both conformations. Previous HDX studies involving the SARS-COV-2 Spike protein did not describe this behavior (27, 28).

Previous studies of the SARS-COV-2 Spike protein by HDX-MS yet have not reported the presence of bimodal mass distributions for any peptides (27, 28). Many of the areas found by the inventors to be obvious peptides with bimodal mass distributions lacked peptide coverage in the study described in (28), and, more specifically, lacked peptides long enough to observe bimodal mass distributions. For example, one of the bimodal peptides observed by the inventors. amino acid residues 878-902 (a peptide of 24 amino acid residues with 21 exchangeable amides), is long enough to provide two very distinct mass distributions, one centered around approximately 9 added Daltons and the other centered around 16 Daltons. This 7 Dalton separation was well observed by the inventors. On the other hand, in (28), the longest peptide described in the same region was amino acid residues 878-893 (only 15 amino acid residues with 13 exchangeable sites).

The inventors also observed the above which appeared bimodal, but the difference was less distinct, with the lighter distribution centered around 6.5 Daltons and the heavier distribution is centered around 10.4 Daltons. The 4 Dalton difference is quite small and can make the bimodal mass distributions appear more like a skewed unimodal mass distribution. Furthermore, differences in back exchange and theoretical maximum deuteration may further obscure the two distributions. The back-exchange observed by the inventors was estimated to be an average of 25% among peptides. In contrast, the authors of (28) estimated their back-exchange to be an average of 34% back-exchange. If the authors of (28) analyzed the peptide described above with 34% back-exchange, they observed the lighter peak around 6 and the heavier peak around 9 Daltons, further reducing the difference in center of the peaks to only 3 Daltons. As a result, the peptide was probably observed as a single unimodal distribution. Finally, the construct in (28) is different from the ones the inventors used in this study, notably it appears to not be glycosylated. Similarly, in (27), where the authors reported their 3 A3 antibody, the authors also do not report bimodal mass distributions for any peptides, even though the binding epitope of the antibody is a peptide that the inventors found with a bimodal mass distribution. The inventors believe that the lack of coverage, lack of long peptides and differences in back-exchange is the primary reason for bimodal mass distribution not observed in (27).

### X. EXAMPLE 4: INTERCONVERSION BETWEEN THE TWO CONFORMATIONS

Based on the HDX-MS data discussed in the present disclosure, the inventors discovered that SARS-COV-2 Spike protein populates two conformations within the pre-fusion state: the classical prefusion structure seen in cryo-EM (herein referred to as state A); and an “alternative” conformation (herein referred to as state B), in which each domain has a similar protomer topology to state A, but more flexible and/or exposed open-trimer interface. The data obtained by the inventors suggest that any potential interconversion between the two states must be slower than the four-hour hydrogen-exchange experiment. Since the transition of the RBD between the “up” and “down” conformation, occurs on the order of seconds, this conformational heterogeneity is not the source of the bimodal distributions observed by the inventors. The hydrogen exchange observed by the inventors reports on the average of the RBD in each of the “up” and “down” states. There are several irreversible situations that may account for conformational heterogeneity, such as differences in glycosylation, proteolytic degradation, irreversible misfolding, or aggregation. To rule these out, the inventors tested whether the two conformations (A and B) interconverted reversibly. Utilizing a pulsed-labeling approach, the inventors monitored the bimodal peptides to quantify the population of each conformation under different conditions. The inventors used the bimodal peptides to quantify the population of each conformation under differing conditions (such as temperature, time, ligand, etc.). Under each condition, the inventors carried out a one-minute pulse of hydrogen exchange and integrated the area under the two mass envelopes for a single bimodal peptide to ascertain the fraction of each conformation under that condition or moment in time (see Example 1). For every condition tested, irrespective of the A:B ratio, all of the peptides examined resulted in the same fractional population for each conformation, indicating that all of these data can be best described as a variable mixture of just two conformations: the canonical pre-fusion conformation A and the newly observed unexpected alternative conformation B.

Long-term incubation (five days, 25° C., pH 7.4) demonstrated a slow shift in population from a majority in the canonical pre-fusion state (state A) to a majority in the alternative conformation (state B). This shift is illustrated in FIG. 2B, in which Gaussian with the higher m/z centroid (2) represents the population of state B, and Gaussian with the lower m/z centroid represents the population of state A (1). At 25° C., the population of state B (represented by the area under state B Gaussian (2)) increased over 4 days of incubation, while the population of state A (represented by the area under state A Gaussian (1)) decreased. Thus, the canonical pre-fusion state A can transform into the alternative state B, and the bimodal behavior cannot be due to sample heterogeneity, such as differential glycosylation. The observed conversion from A to B, however, does not rule out an irreversible process such as degradation or misfolding.

Postulating that the bimodal peaks represent a reversible structural transition, the inventors used temperature to perturb the system and investigate the ability of conformations A and B to interconvert. It was observed that conformations A and B interconverted reversibly, with a preference for B at 4° C. and A at 37° C. As illustrated in FIG. 2B, the population of state B (represented by the area under state B Gaussian (2)) increased over 6 days of incubation at 4° C., while the population of state A (represented by the area under state A Gaussian (1)) decreased. In contrast, the population of state B (represented by the area under state B Gaussian (2)) decreased over 6 days of incubation at 37° C., while the population of state A (represented by the area under state A Gaussian (1)) increased. The kinetics of interconversion were extremely slow: A→B t_{1/2} of ˜17 hours at 4° C. and, when that same sample is moved to ˜37° C., B→A t_{1/2} of ˜9 hours (FIG. 2C, Table 1). Notably, the final distribution at either temperature showed an observable population of both states, indicating a very small energetic difference between the two conformations.

The canonical pre-fusion conformation of SARS-COV-2 Spike protein was previously noted to be temperature dependent. Cryo-EM studies of SARS-COV-2 Spike protein incubated at 4° C. for 5 to 7 days showed less than 10% of the definable pre-fusion particles seen on grids of freshly prepared SARS-COV-2 Spike protein. Incubating SARS-COV-2 Spike protein at 37° C. for three hours after storage at 4° C. recovered particle density to the level seen using freshly prepared protein (29). Failure to detect particles also correlated with a loss in recognition by an antibody known to recognize quaternary structure. These studies are consistent with the findings described in the present disclosure - long-term incubation of Spike at 37° C. biases to the canonical pre-fusion conformation A, while long-term incubation at 4° C. prefers a newly discovered conformation B, which is apparently fails to be visualized on cryo-EM grids.

### XI. EXAMPLE 5: EFFECT OF SEQUENCE CHANGES

### A. HexaPro.

The small energy difference between states A and B of SARS-COV-2 Spike protein indicates that small changes in sequence may affect the relative populations and/or rates of interconversion between them. Indeed, the S-2P variant was designed to stabilize the pre-fusion conformation avoiding spontaneous conversion to the post-fusion form. S-2P is the basis for most currently employed vaccines. Recently, a new version of SARS-COV-2 Spike protein was constructed, termed HexaPro or S-6P, which contains four additional proline mutations designed to increase the apparent stability of the pre-fusion state and improve cellular expression (30).

Using the same HDX-MS process as used for S-2P, HexaPro showed the same bimodal behavior as S2-P, with the same regions reporting on the two conformations (see FIG. 2A, FIG. 9B). At 4° C., HexaPro, similarly to S-2P, converted to state B, but with slower kinetics (t_{1/2} of ˜6 days). At 37° C., HexaPro shifted back to state A with a t_{1/2} of ˜2 hours (FIG. 2C). In sum, HexaPro showed a bias towards the pre-fusion conformation, but also sowed both states A and B populated under all conditions, which his consistent with two low-energy conformations. Importantly, the differences observed between S-2P and HexaPro demonstrated how a small number of mutations can perturb and modulate the conformational landscape of SARS-COV-2 Spike protein, suggesting that the evolving sequence variants may show differences in this conformational exchange (see the discussion further in this disclosure).

### B. An Interprotomer Disulfide-Locked Variant.

To further probe the structural features of the B conformation, the inventors turned to a variant of HexaPro engineered to contain a disulfide bond. This variant trimer contains three disulfide bonds (S383C/D985C) that reach across protomers and lock the RBDs in the down state (31). The inventors found that, when probed by HDX-MS, this disulfide-locked variant remained completely in the A state and did not show any observable population of the B state, even after overnight incubation at 25° C. These observation are consistent with a model, in which formation of the B state requires opening of the inter-protomer (trimer) interface and exposure of the RBDs, which would be prohibited by the interprotomer crosslinks in the disulfide-locked variant.

UK Variant.

Increasingly infective SARS-COV-2 variants of concern are being discovered throughout the global population on a regular basis. Most of these variants of concern include mutations in the SARS-COV-2 Spike protein, primarily in the S1 domain. Some of these mutations reside in the ACE2-interaction surface, others do not. Therefore, the inventors asked if these mutations can influence the biases and kinetics of interconversion between the A and B conformations. The inventors monitored the A/B conversion for a variant of HexaPro that includes all but one of the S1 mutations in the B.1.1.7 variant, which originated in the United Kingdom (Δ69-70 (NTD), Δ145 (NTD), N501Y(RBD), A570D (SD1), P681H (SD2)), termed UK S1 HexaPro. In comparison to HexaPro, UK S1 HexaPro showed notable differences in both the relative preference for state B and the kinetics of interconversion. At 4° C., UK S1 HexaPro converted to state B nearly 20 times faster than HexaPro (FIG. 2C,

Table 1). Furthermore, UK S1 HexaPro showed no detectable pre-fusion conformer at 4° C., while HexaPro showed at least 30% even after several weeks at 4° C. At 37° C., the kinetics and equilibrium distribution appeared nearly identical between the two proteins. All of the mutations in B.1.1.7 (and, consequently, in UK S1 HexaPro) are at solvent-exposed amino acid residues, except amino acid residue 570, which contacts the S2 subunit and resides in a region with observed bimodal behavior. Thus, despite their location in the S1 subunit and not at the core trimer interface, these specific B.1.1.7 mutations allosterically affect the interconversion between states A and B.

### XII. EXAMPLE 6: EFFECTS OF ACE2 BINDING

The primary function of the RBD of SARS-COV-2 Spike protein is to recognize the host cell receptor ACE2. In the down conformation, the RBD is occluded from binding to ACE2, and in the up conformation it is accessible. The entire trimer can exist with zero, one, two, or all three RBDs in the up conformation (7, 15). In the isolated RBD, the Receptor Binding Motif (RBM) should always be accessible for ACE2 binding. The inventors used HDX to monitor the binding of ACE2 to isolated RBD and to RBD in full-length S-2P. In these experiments, used a soluble dimeric form of ACE2 (ACE2-Fc, herein referred to as ACE2). For isolated RBD (amino acid residues 319-541, see Example 1), the inventors obtained 141 peptides, including one glycosylated peptide spanning the N-glycosylation site at amino acid residue 343 (no peptides are observed for site 331), resulting in 82% sequence coverage with an average redundancy of 8 (FIG. 6).

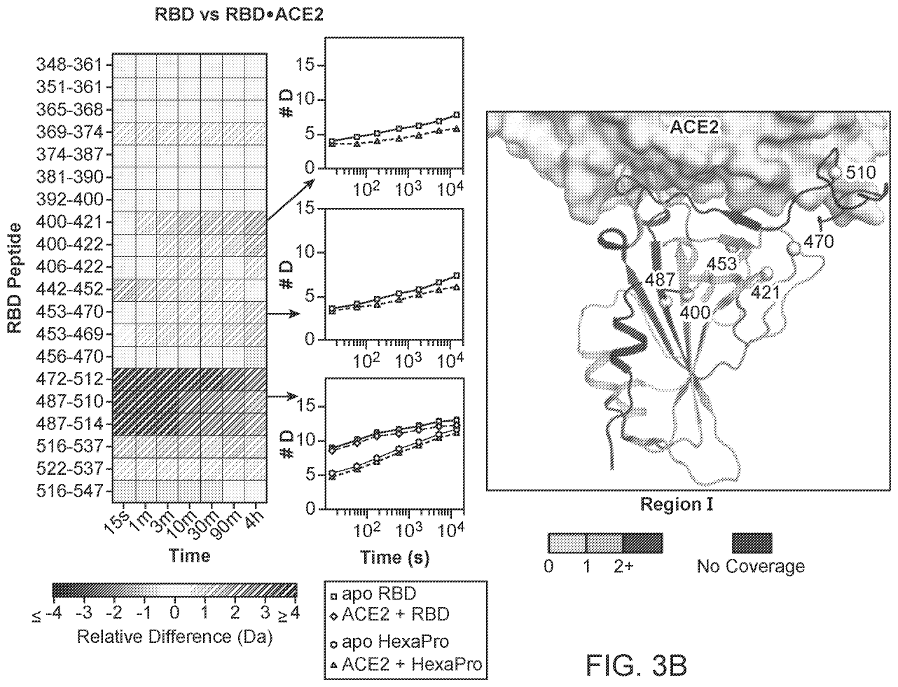

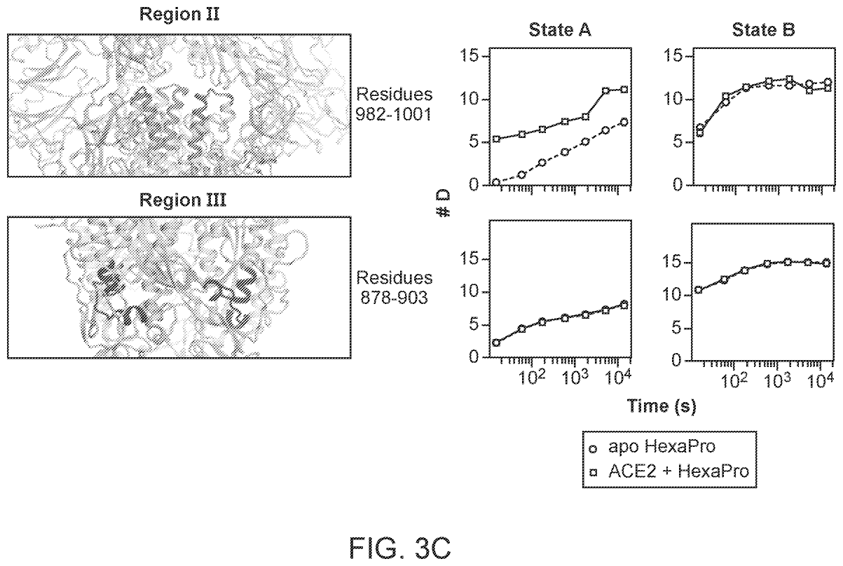

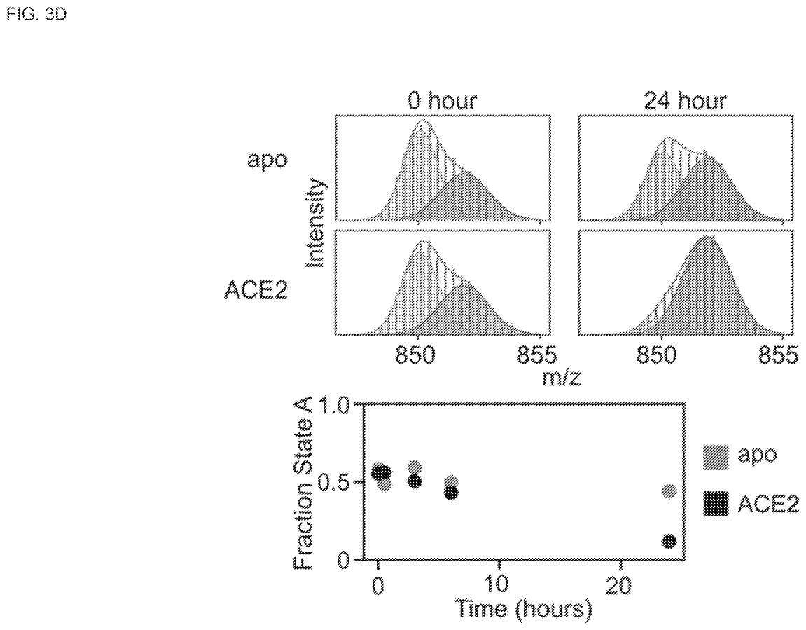

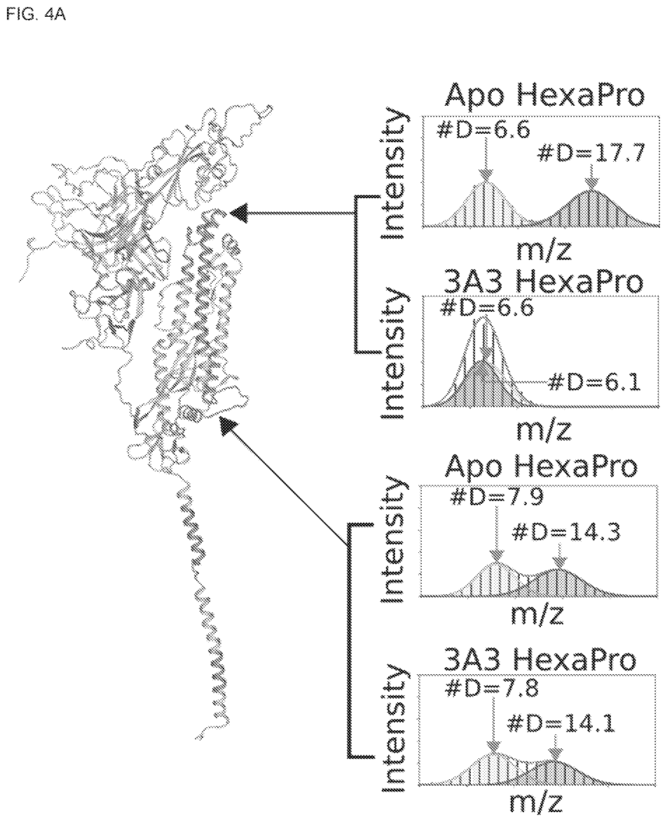

The effects of ACE2 binding are illustrated in FIG. 3A-3D. In the presence of ACE2, the latter half of the RBM (amino acid residues 472-513) showed a notable decrease in hydrogen exchange upon binding ACE2 (FIG. 3B), which is consistent with the known ACE2/RBD binding interface (32, 33). The inventors also observed small, but significant, changes for other regions near the binding interface. Importantly, the inventors observed very similar changes in HDX rates in RBD in both the isolated RBD and RBD in full-length SP-2 trimer, suggesting that all three RBDs in full-length SP-2 interacted with ACE2, and that both the A and the B state can productively bind ACE2, which for the pre-fusion (A) state requires the RBD transitioning to the up state.

In the context of full-length SP-2, the inventors also observed notable changes

outside of the RBD, particularly in state A, where a few peptides exchange more rapidly in the presence of ACE2 (in state B these peptides do not have any notable difference in the presence of ACE2) (FIG. 3C). These peptides are located on the top of S2 (amino acid residues 978-1001), a region known to become more exposed when RBD transitions from a down to an up conformation. Since ACE2 binding in the pre-fusion state requires the RBDs to be in the up conformation, this increased exchange reflects the known biases in the RBD conformation - a pre-fusion state with RBDs primarily in the down conformation, which must transition to an up conformation to bind ACE2. The inventors also observed changes in the interconversion between state A and state B in the presence of ACE2, such that state B is more preferred (FIG. 3D).

### XIII. EXAMPLE 7: RBD DYNAMICS

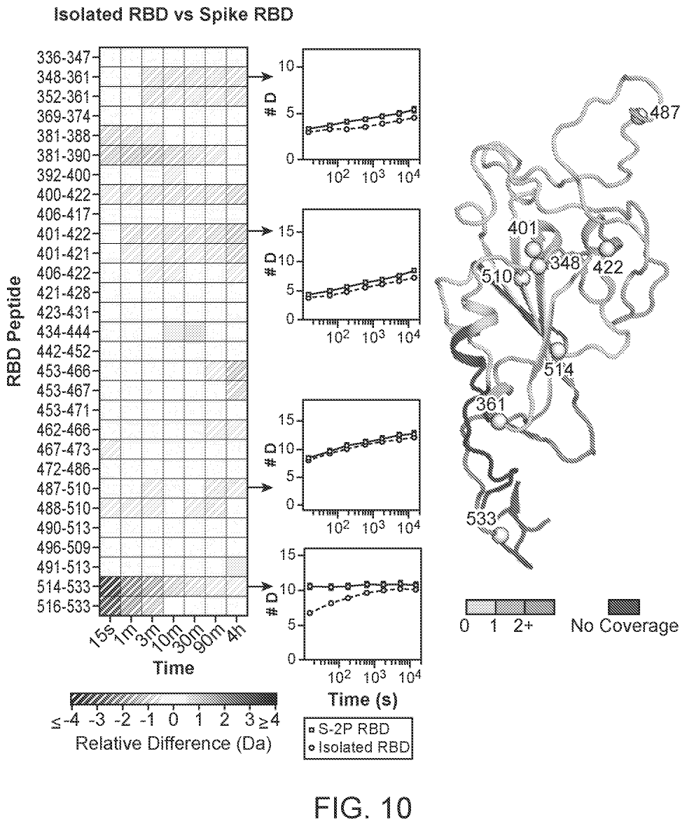

The isolated RBD of SARS-COV-2 Spike protein has been used for many biochemical studies and is the main component of many clinical diagnostic approaches. It is therefore important to ask whether there are large differences in the RBD when it is found in isolation, as compared to RBD found in SARS-COV-2 Spike protein trimer. The experiments conducted by the inventors allowed for the comparison between the isolated RBD and the RBD in SP-2 trimer. Very few peptides in the RBD showed substantial changes in HDX behavior between the two proteins (FIG. 10). These results support the use of approaches such as deep sequence mutagenesis on the isolated RBD to gain information on the potential effects of variants, such as escape mutations (34).

The inventors observed some key differences, however, mostly at the termini of the isolated RBD, in the expected interactions with the rest of SARS-COV-2 Spike protein , and across the protomer interface. The C-terminal region of the RBD (amino acid residues 516-537) was notably less protected in the isolated RBD than in SP-2. This region is not part of the RBD globular domain, and, in full-length SARS-COV-2 Spike protein, forms part of subdomain 1, which is consisted with an increase in flexibility of this region when RBD is isolated from the rest of the subdomain. Future studies with the isolated RBD may benefit from removal of both C-terminal and N-terminal regions, as they are likely disordered and may interfere with crystallization or lead to increases in aggregation.

### XIV. EXAMPLE 8: 3A3 ANTIBODY BINDS SPECIFICALLY TO THE B STATE

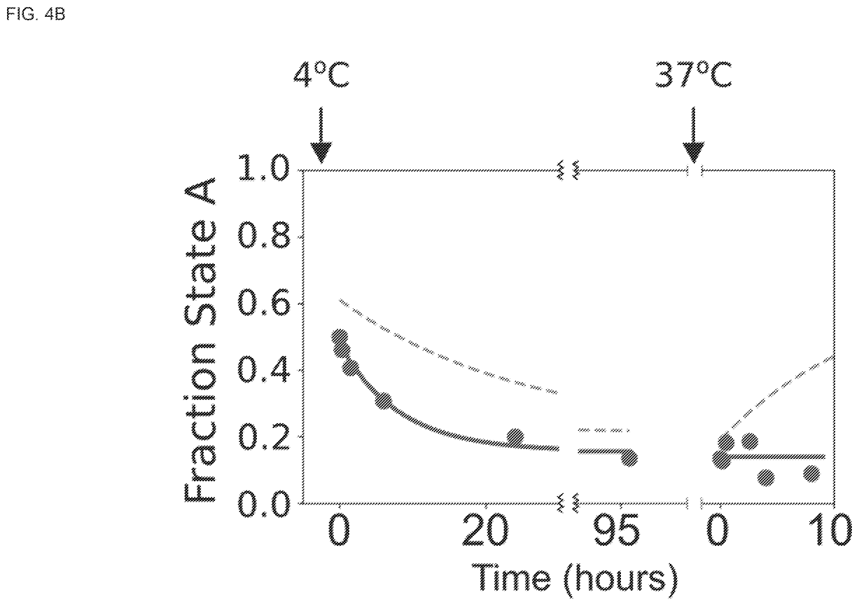

Recently, 3A3 antibody was developed that binds to MERS-COV, SARS-COV-1, and SARS-COV-2, with an apparent epitope in a region where the inventors observed bimodal peptide behavior (amino acid residues ˜980-1000) (27). This region, however, is inaccessible in the pre-fusion structure of SARS-COV-2 Spike protein . The region is buried in the pre-fusion structure, when all RBDs are down, and highly occluded when the RBDs are up. The HDX data obtained by the inventors indicated that this region was exposed in state B. To confirm the exposure of the 3A3 epitope in state B, the inventors conducted the HDX studies in the presence of 3A3 antibody and observed strong increased protection in the 978-1001 region. Moreover, this protection was directly associated with state B. In state B, 3A3 antibody epitope was occluded from solvent and showed similar exchange in both A and B states. These data (illustrated in FIG. 4A and FIG. 4B) suggested a model, in which 3A3 antibody binds uniquely to B state of SARS-COV-2 Spike protein.

To confirm this hypothesis, the inventors looked at the effect of 3A3 antibody binding on the temperature-induced conversion between A and B states. 3A3 antibody increased the rate of conversion from A to B at 4° C., decreasing t_{1/2} from ˜17 hours to ˜5 hours (FIG. 4). This increase in the observed rate implied that 3A3 antibody also affected the transition state for the conversion. Furthermore, returning the sample to 37° C. in the presence of 3A3 antibody (state B saturated with 3A3 antibody) prohibited any transition back to the pre-fusion state, indicating that the binding of 3A3 antibody prevented formation of the pre-fusion state, most likely due to steric hindrance of the antibody being bound to the trimer interface. Since 3A3 antibody binds both wild-type and D614G SARS-COV-2 Spike proteins, when expressed on the surface of cells, and neutralizes pseudovirus expressing these SARS-COV-2 Spike proteins (27), the data obtained by the inventors shows that state B exposes broadly neutralization-sensitive epitopes. This is information is useful for development of therapeutics and vaccines.

### XV. EXAMPLE 9: STRUCTURAL MODEL FOR STATE B

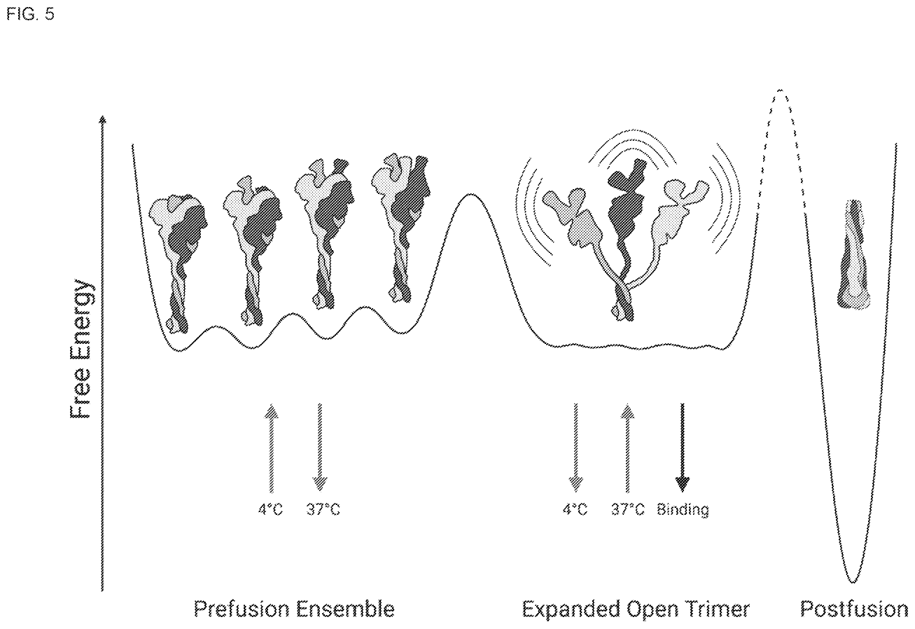

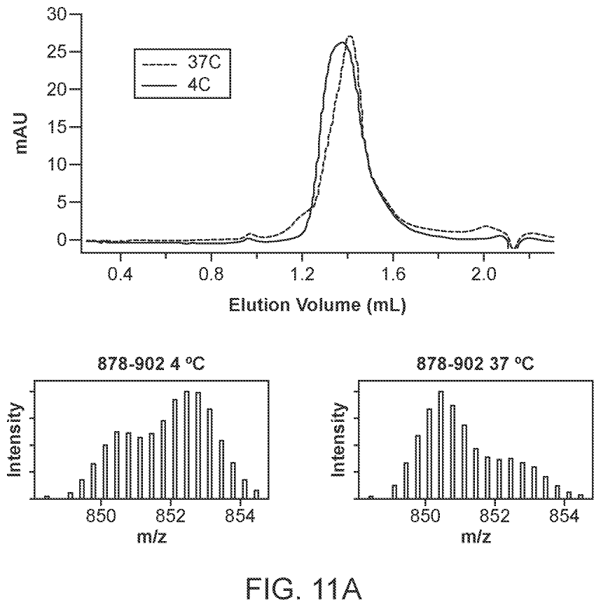

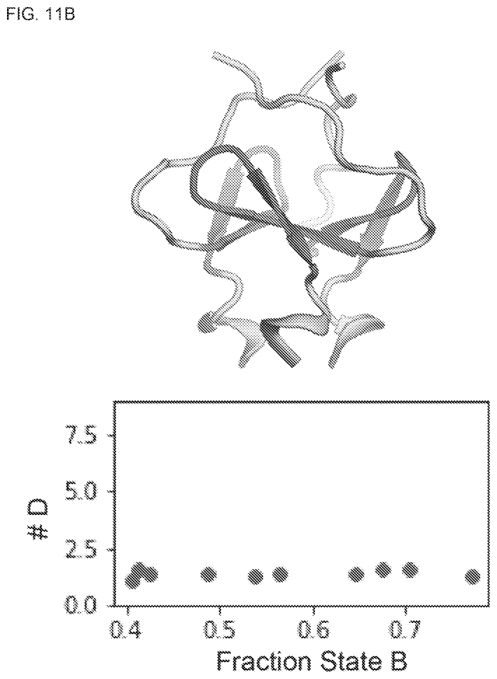

The above-discussed data allowed the inventors to create a structural model for state B (FIG. 5). The overall fold, or topology, of each domain in state B is likely similar to the pre-fusion structure as, with the exception of the bimodal peptides, their hydrogen exchange patterns are similar. The bimodal peptides, which report on the two different conformations, cluster in the trimer interface, suggesting that this interface is more accessible to solvent in state B than in state A. State B is not a monomer. Size-exclusion chromatography and the hydrogen exchange data at the trimerization motif, confirm that both conformations A and B are trimeric (FIG. 11A and FIG. 11B). In these soluble ectodomain constructs, the trimer is held together by the appended C-terminal trimerization domain, while in the full-length native SARS-COV-2 Spike protein trimer, the transmembrane helical segment likely serves this function. Therefore, state B is best modeled as an opened-up trimer with three protomers with domains that are structurally uncoupled. An ensemble of opened-up trimers with heterogeneous positioning of the protomers best explains the lack of cryo-EM data on state B. An opened-up class 1 viral fusion protein has been reported for respiratory syncytial virus (RSV) and visualized by a low resolution structure (35). This structural data from RSV and reports of an opening up of other viral fusion proteins (36, 37) support a model of an ensemble of open-trimers with various degrees of openness.

A loss of interprotomer contacts in state B implies that, in state B, RBDs no longer contact adjacent protomers, and thus do not have distinct “down” and “up” conformations. Rather, in state B, RBDs are likely always in a binding-competent state, perhaps even more accessible than om the canonical “up” state. This increased availability of the RBDs in state B may drive a preference for the B state in the presence of ACE2. Furthermore, in the canonical pre-fusion conformation (state A), having all three RBDs bound to ACE2 may lead to steric hindrances, but, in state B, all three RBDs should be able to bind ACE2 with high affinity. Interestingly, mutations found in variants of concern, such as in the UK HexaPro variant, greatly increase the rate of conversion to state B, which may play a role in the noted increased infectivity.

Molecular dynamics have shown a smaller opening of the SARS-COV-2 Spike protein where an RBD and adjacent NTD twist and peel away from the center of the SARS-COV-2 Spike protein, revealing a cryptic epitope at the top of the S2 domain (20). This rapidly sampled conformation is not state B, as it does not involve the S2 trimer interface, and the timescale of conversion to state B is unlikely to be sampled during a molecular dynamics simulation. This partial opening, however, may be on a pathway to state B.

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

TABLE 1
Rates of interconversion between the A (prefusion) and B (open
trimer) conformations of SARS-CoV-2 Spike protein ectodomains.

| Temperature | Protein | k_{observed} (hr⁻¹)* | t_{1/2} (hours) |
|---|---|---|---|
| 37° C.→4° C. | S-2P | 0.4 | 17 |
| | HexaPro | 0.005 | 143 |
| | UK S1 | 0.2 | 4 |
| | HexaPro | | |
| | S-2P + 3A3 | 0.1 | 5 |
| 37° C.→10° C. | S-2P | 0.5 | 14 |
| | HexaPro | 0.004 | 171 |
| | UK S1 | 0.1, 0.2** | 3, 5** |
| | HexaPro | | |
| 4° C.→37° C. | S-2P | 0.8 | 9 |
| | HexaPro | 0.3 | 2 |
| | UK S1 | 0.2 | 4 |
| | HexaPro | | |

*k_{observed} is the observed rate of change in the population of the A state after a temperature jump. This relaxation rate is the sum of the forward and reverse rates, which is dominated by the major conformational change (A→B at 4° C., 10° C. and B→A at 37° C.). t_{1/2} is the half time for that same rate, ln2/k_{observed}).
**Time course was monitored twice, and the results of each fit are reported.

### XVI. PUBLICATIONS CITED IN THIS DISCLOSURE

1. A. Baum, B. O. Fulton, E. Wloga, R. Copin, K. E. Pascal, V. Russo, S. Giordano, K. Lanza, N. Negron, M. Ni, Y. Wei, G. S. Atwal, A. J. Murphy, N. Stahl, G. D. Yancopoulos, C. A. Kyratsous, Antibody cocktail to SARS-COV-2 spike protein prevents rapid mutational escape seen with individual antibodies. Science. 369, 1014-1018 (2020).
2. P. C. Taylor, A. C. Adams, M. M. Hufford, I. de la Torre, K. Winthrop, R. L. Gottlieb, Neutralizing monoclonal antibodies for treatment of COVID-19. Nat. Rev. Immunol. 21, 382-393 (2021).
3. J. Hansen, A. Baum, K. E. Pascal, V. Russo, S. Giordano, E. Wloga, B. O. Fulton, Y. Yan, K. Koon, K. Patel, K. M. Chung, A. Hermann, E. Ullman, J. Cruz, A. Rafique, T. Huang, J. Fairhurst, C. Libertiny, M. Malbec, W .- Y. Lee, R. Welsh, G. Farr, S. Pennington, D. Deshpande, J. Cheng, A. Watty, P. Bouffard, R. Babb, N. Levenkova, C. Chen, B. Zhang, A. Romero Hernandez, K. Saotome, Y. Zhou, M. Franklin, S. Sivapalasingam, D. C. Lye, S. Weston, J. Logue, R. Haupt, M. Frieman, G. Chen, W. Olson, A. J. Murphy, N. Stahl, G. D. Yancopoulos, C. A. Kyratsous, Studies in humanized mice and convalescent humans yield a SARS-COV-2 antibody cocktail. Science. 369, 1010-1014 (2020).
4. R. Shi, C. Shan, X. Duan, Z. Chen, P. Liu, J. Song, T. Song, X. Bi, C. Han, L. Wu, G. Gao, X. Hu, Y. Zhang, Z. Tong, W. Huang, W. J. Liu, G. Wu, B. Zhang, L. Wang, J. Qi, H. Feng, F .- S. Wang, Q. Wang, G. F. Gao, Z. Yuan, J. Yan, A human neutralizing antibody targets the receptor-binding site of SARS-COV-2. Nature. 584, 120-124 (2020).
5. Y. Watanabe, J. D. Allen, D. Wrapp, J. S. Mclellan, M. Crispin, Site-specific glycan analysis of the SARS-COV-2 spike. Science. 91, eabb9983 (2020).
6. M. Hoffmann, H. Kleine-Weber, S. Schroeder, N. Krüger, T. Herrler, S. Erichsen, T. S. Schiergens, G. Herrler, N .- H. Wu, A. Nitsche, M. A. Müller, C. Drosten, S. Pohlmann, SARS-COV-2 Cell Entry Depends on ACE2 and TMPRSS2 and Is Blocked by a Clinically Proven Protease Inhibitor. Cell. 181, 271-280.e8 (2020).
7. D. J. Benton, A. G. Wrobel, P. Xu, C. Roustan, S. R. Martin, P. B. Rosenthal, J. J. Skehel, S. J. Gamblin, Receptor binding and priming of the spike protein of SARS-COV-2 for membrane fusion. Nature. 588, 327-330 (2020).
8 S. Jiang, C. Hillyer, L. Du, Neutralizing Antibodies against SARS-COV-2 and Other Human Coronaviruses. Trends Immunol. 41, 355-359 (2020).
9. D. Wrapp, N. Wang, K. S. Corbett, J. A. Goldsmith, C .- L. Hsieh, O. Abiona, B. S. Graham, J. S. Mclellan, Cryo-EM structure of the 2019-nCOV spike in the prefusion conformation. Science. 367, 1260-1263 (2020).
10. A. C. Walls, Y .- J. Park, M. A. Tortorici, A. Wall, A. T. McGuire, D. Veesler, Structure, Function, and Antigenicity of the SARS-COV-2 Spike Glycoprotein. Cell. 181, 281-292.e6 (2020).
11. H. M. Berman, J. Westbrook, Z. Feng, G. Gilliland, T. N. Bhat, H. Weissig, I. N. Shindyalov, P. E. Bourne, The Protein Data Bank. Nucleic Acids Res. 28, 235-242 (2000).
12. J. M. White, S. E. Delos, M. Brecher, K. Schornberg, Structures and mechanisms of viral membrane fusion proteins: multiple variations on a common theme. Crit. Rev. Biochem. Mol. Biol. 43, 189-219 (2008).
13. F. Li, Structure, Function, and Evolution of Coronavirus Spike Proteins. Annu Rev Virol. 3, 237-261 (2016).
14. T. Zhou, Y. Tsybovsky, J. Gorman, M. Rapp, G. Cerutti, G .- Y. Chuang, P. S. Katsamba, J. M. Sampson, A. Schön, J. Bimela, J. C. Boyington, A. Nazzari, A. S. Olia, W. Shi, M. Sastry, T. Stephens, J. Stuckey, I .- T. Teng, P. Wang, S. Wang, B. Zhang, R. A. Friesner, D. D. Ho, J. R. Mascola, L. Shapiro, P. D. Kwong, Cryo-EM Structures of SARS-COV-2 Spike without and with ACE2 Reveal a pH-Dependent Switch to Mediate Endosomal Positioning of Receptor-Binding Domains. Cell Host Microbe. 28, 867-879.e5 (2020).
15. T. Xiao, J. Lu, J. Zhang, R. I. Johnson, L. G. A. Mckay, N. Storm, C. L. Lavine, H. Peng, Y. Cai, S. Rits-Volloch, S. Lu, B. D. Quinlan, M. Farzan, M. S. Seaman, A. Griffiths, B. Chen, A trimeric human angiotensin-converting enzyme 2 as an anti-SARS-COV-2 agent. Nat. Struct. Mol. Biol. 28, 202-209 (2021).
16. S. Belouzard, V. C. Chu, G. R. Whittaker, Activation of the SARS coronavirus spike protein via sequential proteolytic cleavage at two distinct sites. Proc. Natl. Acad. Sci. U. S. A. 106, 5871-5876 (2009).
17. Y. Cai, J. Zhang, T. Xiao, H. Peng, S. M. Sterling, R. M. Walsh Jr, S. Rawson, S. Rits-Volloch, B. Chen, Distinct conformational states of SARS-COV-2 spike protein. Science. 369, 1586-1592 (2020).
18. A. C. Walls, M. A. Tortorici, J. Snijder, X. Xiong, B .- J. Bosch, F. A. Rey, D. Veesler, Tectonic conformational changes of a coronavirus spike glycoprotein promote membrane fusion. Proc. Natl. Acad. Sci. U. S. A. 114, 11157-11162 (2017).
19. M. Lu, P. D. Uchil, W. Li, D. Zheng, D. S. Terry, J. Gorman, W. Shi, B. Zhang, T. Zhou, S. Ding, R. Gasser, J. Prevost, G. Beaudoin-Bussières, S. P. Anand, A. Laumaea, J. R. Grover, L. Liu, D. D. Ho, J. R. Mascola, A. Finzi, P. D. Kwong, S. C. Blanchard, W. Mothes, Real-Time Conformational Dynamics of SARS-COV-2 Spikes on Virus Particles. Cell Host Microbe. 28, 880-891.e8 (2020).
20. M. I. Zimmerman, J. R. Porter, M. D. Ward, S. Singh, N. Vithani, A. Meller, U. L. Mallimadugula, C. E. Kuhn, J. H. Borowsky, R. P. Wiewiora, M. F. D. Hurley, A. M. Harbison, C. A. Fogarty, J. E. Coffland, E. Fadda, V. A. Voelz, J. D. Chodera, G. R. Bowman, SARS-COV-2 simulations go exascale to predict dramatic spike opening and cryptic pockets across the proteome. Nat. Chem. 13, 651-659 (2021).
21. T. E. Sztain, S .- H. Ahn, A. T. Bogetti, L. Casalino, J. A. Goldsmith, R. S. McCool, F. L. Kearns, J. Andrew McCammon, J. S. Mclellan, L. Chong, R. E. Amaro, A glycan gate controls opening of the SARS-COV-2 spike protein. Cold Spring Harbor Laboratory (2021), p. 2021.2.15.431212, , doi: 10.1101/2021.2.15.431212.
22. S. W. Englander, Hydrogen exchange and mass spectrometry: A historical perspective. J. Am. Soc. Mass Spectrom. 17, 1481-1489 (2006).
23. J. Zheng, T. Strutzenberg, B. D. Pascal, P. R. Griffin, Protein dynamics and conformational changes explored by hydrogen/deuterium exchange mass spectrometry. Curr. Opin. Struct. Biol. 58, 305-313 (2019).
24. L. Casalino, Z. Gaieb, J. A. Goldsmith, C. K. Hjorth, A. C. Dommer, A. M. Harbison, C. A. Fogarty, E. P. Barros, B. C. Taylor, J. S. Mclellan, E. Fadda, R. E. Amaro, Beyond Shielding: The Roles of Glycans in the SARS-COV-2 Spike Protein. ACS Cent. Sci. 6, 1722-1734 (2020).
25. B. Turoňová, M. Sikora, C. Schürmann, W. J. H. Hagen, S. Welsch, F. E. C. Blanc, S. von Bülow, M. Gecht, K. Bagola, C. Hörner, G. van Zandbergen, J. Landry, N.T.D de Azevedo, S. Mosalaganti, A. Schwarz, R. Covino, M. D. Mühlebach, G. Hummer, J. K. Locker, M. Beck, In situ structural analysis of SARS-COV-2 spike reveals flexibility mediated by three hinges. Science. 370, 203-208 (2020).
26. C. O. Barnes, A. P. West Jr, K. E. Huey-Tubman, M. A. G. Hoffmann, N. G. Sharaf, P. R. Hoffman, N. Koranda, H. B. Gristick, C. Gaebler, F. Muecksch, J. C. C. Lorenzi, S. Finkin, T. Hägglöf, A. Hurley, K. G. Millard, Y. Weisblum, F. Schmidt, T. Hatziioannou, P. D. Bieniasz, M. Caskey, D. F. Robbiani, M. C. Nussenzweig, P. J. Bjorkman, Structures of Human Antibodies Bound to SARS-COV-2 Spike Reveal Common Epitopes and Recurrent Features of Antibodies. Cell. 182, 828-842.e16 (2020).
27. Y. Huang, A. W. Nguyen, C .- L. Hsieh, R. Silva, O. S. Olaluwoye, R. E. Wilen, T. S. Kaoud, L. R. Azouz, A. N. Qerqez, K. C. Le, A. L. Bohanon, A. M. DiVenere, Y. Liu, A. G. Lee, D. Amengor, K. N. Dalby, S. D′Arcy, J. S. McLellan, J. A. Maynard, Identification of a conserved neutralizing epitope present on spike proteins from all highly pathogenic coronaviruses. bioRxiv (2021), p. 2021.01.31.428824, , doi: 10.1101/2021.01.31.428824.
28. P. V. Raghuvamsi, N. K. Tulsian, F. Samsudin, X. Qian, K. Purushotorman, G. Yue, M. M. Kozma, W. Y. Hwa, J. Lescar, P. J. Bond, P. A. MacAry, G. S. Anand, SARS-COV-2 S protein: ACE2 interaction reveals novel allosteric targets. Elife. 10 (2021), doi: 10.7554/eLife.63646.
1. A. Baum, B. O. Fulton, E. Wloga, R. Copin, K. E. Pascal, V. Russo, S. Giordano, K. Lanza, N. Negron, M. Ni, Y. Wei, G. S. Atwal, A. J. Murphy, N. Stahl, G. D. Yancopoulos, C. A. Kyratsous, Antibody cocktail to SARS-COV-2 spike protein prevents rapid mutational escape seen with individual antibodies. Science. 369, 1014-1018 (2020).
2. P. C. Taylor, A. C. Adams, M. M. Hufford, I. de la Torre, K. Winthrop, R. L. Gottlieb, Neutralizing monoclonal antibodies for treatment of COVID-19. Nat. Rev. Immunol. 21, 382-393 (2021).
3. J. Hansen, A. Baum, K. E. Pascal, V. Russo, S. Giordano, E. Wloga, B. O. Fulton, Y. Yan, K. Koon, K. Patel, K. M. Chung, A. Hermann, E. Ullman, J. Cruz, A. Rafique, T. Huang, J. Fairhurst, C. Libertiny, M. Malbec, W .- Y. Lee, R. Welsh, G. Farr, S. Pennington, D. Deshpande, J. Cheng, A. Watty, P. Bouffard, R. Babb, N. Levenkova, C. Chen, B. Zhang, A. Romero Hernandez, K. Saotome, Y. Zhou, M. Franklin, S. Sivapalasingam, D. C. Lye, S. Weston, J. Logue, R. Haupt, M. Frieman, G. Chen, W. Olson, A. J. Murphy, N. Stahl, G. D. Yancopoulos, C. A. Kyratsous, Studies in humanized mice and convalescent humans yield a SARS-COV-2 antibody cocktail. Science. 369, 1010-1014 (2020).
4. R. Shi, C. Shan, X. Duan, Z. Chen, P. Liu, J. Song, T. Song, X. Bi, C. Han, L. Wu, G. Gao, X. Hu, Y. Zhang, Z. Tong, W. Huang, W. J. Liu, G. Wu, B. Zhang, L. Wang, J. Qi, H. Feng, F .- S. Wang, Q. Wang, G. F. Gao, Z. Yuan, J. Yan, A human neutralizing antibody targets the receptor-binding site of SARS-COV-2. Nature. 584, 120-124 (2020).
5. Y. Watanabe, J. D. Allen, D. Wrapp, J. S. Mclellan, M. Crispin, Site-specific glycan analysis of the SARS-COV-2 spike. Science. 91, eabb9983 (2020).
6. M. Hoffmann, H. Kleine-Weber, S. Schroeder, N. Krüger, T. Herrler, S. Erichsen, T. S. Schiergens, G. Herrler, N .- H. Wu, A. Nitsche, M. A. Müller, C. Drosten, S. Pöhlmann, SARS-COV-2 Cell Entry Depends on ACE2 and TMPRSS2 and Is Blocked by a Clinically Proven Protease Inhibitor. Cell. 181, 271-280.e8 (2020).
7. D. J. Benton, A. G. Wrobel, P. Xu, C. Roustan, S. R. Martin, P. B. Rosenthal, J. J. Skehel, S. J. Gamblin, Receptor binding and priming of the spike protein of SARS-COV-2 for membrane fusion. Nature. 588, 327-330 (2020).
8. S. Jiang, C. Hillyer, L. Du, Neutralizing Antibodies against SARS-COV-2 and Other Human Coronaviruses. Trends Immunol. 41, 355-359 (2020).
9. D. Wrapp, N. Wang, K. S. Corbett, J. A. Goldsmith, C .- L. Hsieh, O. Abiona, B. S. Graham, J. S. Mclellan, Cryo-EM structure of the 2019-nCOV spike in the prefusion conformation. Science. 367, 1260-1263 (2020).
10. A. C. Walls, Y .- J. Park, M. A. Tortorici, A. Wall, A. T. McGuire, D. Veesler, Structure, Function, and Antigenicity of the SARS-COV-2 Spike Glycoprotein. Cell. 181, 281-292.e6 (2020).
11. H. M. Berman, J. Westbrook, Z. Feng, G. Gilliland, T. N. Bhat, H. Weissig, I. N. Shindyalov, P. E. Bourne, The Protein Data Bank. Nucleic Acids Res. 28, 235-242 (2000).
12. J. M. White, S. E. Delos, M. Brecher, K. Schornberg, Structures and mechanisms of viral membrane fusion proteins: multiple variations on a common theme. Crit. Rev. Biochem. Mol. Biol. 43, 189-219 (2008).
13. F. Li, Structure, Function, and Evolution of Coronavirus Spike Proteins. Annu Rev Virol. 3, 237-261 (2016).
14. T. Zhou, Y. Tsybovsky, J. Gorman, M. Rapp, G. Cerutti, G .- Y. Chuang, P. S. Katsamba, J. M. Sampson, A. Schön, J. Bimela, J. C. Boyington, A. Nazzari, A. S. Olia, W. Shi, M. Sastry, T. Stephens, J. Stuckey, I .- T. Teng, P. Wang, S. Wang, B. Zhang, R. A. Friesner, D. D. Ho, J. R. Mascola, L. Shapiro, P. D. Kwong, Cryo-EM Structures of SARS-COV-2 Spike without and with ACE2 Reveal a pH-Dependent Switch to Mediate Endosomal Positioning of Receptor-Binding Domains. Cell Host Microbe. 28, 867-879.e5 (2020).
15. T. Xiao, J. Lu, J. Zhang, R. I. Johnson, L. G. A. Mckay, N. Storm, C. L. Lavine, H. Peng, Y. Cai, S. Rits-Volloch, S. Lu, B. D. Quinlan, M. Farzan, M. S. Seaman, A. Griffiths, B. Chen, A trimeric human angiotensin-converting enzyme 2 as an anti-SARS-COV-2 agent. Nat. Struct. Mol. Biol. 28, 202-209 (2021).
16. S. Belouzard, V. C. Chu, G. R. Whittaker, Activation of the SARS coronavirus spike protein via sequential proteolytic cleavage at two distinct sites. Proc. Natl. Acad. Sci. U. S. A. 106, 5871-5876 (2009).
17. Y. Cai, J. Zhang, T. Xiao, H. Peng, S. M. Sterling, R. M. Walsh Jr, S. Rawson, S. Rits-Volloch, B. Chen, Distinct conformational states of SARS-COV-2 spike protein. Science. 369, 1586-1592 (2020).
18. A. C. Walls, M. A. Tortorici, J. Snijder, X. Xiong, B .- J. Bosch, F. A. Rey, D. Veesler, Tectonic conformational changes of a coronavirus spike glycoprotein promote membrane fusion. Proc. Natl. Acad. Sci. U. S. A. 114, 11157-11162 (2017).
19. M. Lu, P. D. Uchil, W. Li, D. Zheng, D. S. Terry, J. Gorman, W. Shi, B. Zhang, T. Zhou, S. Ding, R. Gasser, J. Prevost, G. Beaudoin-Bussières, S. P. Anand, A. Laumaea, J. R. Grover, L. Liu, D. D. Ho, J. R. Mascola, A. Finzi, P. D. Kwong, S. C. Blanchard, W. Mothes, Real-Time Conformational Dynamics of SARS-COV-2 Spikes on Virus Particles. Cell Host Microbe. 28, 880-891.e8 (2020).
20. M. I. Zimmerman, J. R. Porter, M. D. Ward, S. Singh, N. Vithani, A. Meller, U. L. Mallimadugula, C. E. Kuhn, J. H. Borowsky, R. P. Wiewiora, M. F. D. Hurley, A. M. Harbison, C. A. Fogarty, J. E. Coffland, E. Fadda, V. A. Voelz, J. D. Chodera, G. R. Bowman, SARS-COV-2 simulations go exascale to predict dramatic spike opening and cryptic pockets across the proteome. Nat. Chem. 13, 651-659 (2021).
21. T. E. Sztain, S .- H. Ahn, A. T. Bogetti, L. Casalino, J. A. Goldsmith, R. S. McCool, F. L. Kearns, J. Andrew McCammon, J. S. Mclellan, L. Chong, R. E. Amaro, A glycan gate controls opening of the SARS-COV-2 spike protein. Cold Spring Harbor Laboratory (2021), p. 2021.2.15.431212, , doi: 10.1101/2021.2.15.431212.
22. S. W. Englander, Hydrogen exchange and mass spectrometry: A historical perspective. J. Am. Soc. Mass Spectrom. 17, 1481-1489 (2006).
23. J. Zheng, T. Strutzenberg, B. D. Pascal, P. R. Griffin, Protein dynamics and conformational changes explored by hydrogen/deuterium exchange mass spectrometry. Curr. Opin. Struct. Biol. 58, 305-313 (2019).
24. L. Casalino, Z. Gaieb, J. A. Goldsmith, C. K. Hjorth, A. C. Dommer, A. M. Harbison, C. A. Fogarty, E. P. Barros, B. C. Taylor, J. S. Mclellan, E. Fadda, R. E. Amaro, Beyond Shielding: The Roles of Glycans in the SARS-COV-2 Spike Protein. ACS Cent. Sci. 6, 1722-1734 (2020).
25. B. Turoňová, M. Sikora, C. Schürmann, W. J. H. Hagen, S. Welsch, F. E. C. Blanc, S. von Bülow, M. Gecht, K. Bagola, C. Hörner, G. van Zandbergen, J. Landry, N.T.D de Azevedo, S. Mosalaganti, A. Schwarz, R. Covino, M. D. Mühlebach, G. Hummer, J. K. Locker, M. Beck, In situ structural analysis of SARS-COV-2 spike reveals flexibility mediated by three hinges. Science. 370, 203-208 (2020).
26. C. O. Barnes, A. P. West Jr, K. E. Huey-Tubman, M. A. G. Hoffmann, N. G. Sharaf, P. R. Hoffman, N. Koranda, H. B. Gristick, C. Gaebler, F. Muecksch, J. C. C. Lorenzi, S. Finkin, T. Hägglöf, A. Hurley, K. G. Millard, Y. Weisblum, F. Schmidt, T. Hatziioannou, P. D. Bieniasz, M. Caskey, D. F. Robbiani, M. C. Nussenzweig, P. J. Bjorkman, Structures of Human Antibodies Bound to SARS-COV-2 Spike Reveal Common Epitopes and Recurrent Features of Antibodies. Cell. 182, 828-842.e16 (2020).
27. Y. Huang, A. W. Nguyen, C .- L. Hsieh, R. Silva, O. S. Olaluwoye, R. E. Wilen, T. S. Kaoud, L. R. Azouz, A. N. Qerqez, K. C. Le, A. L. Bohanon, A. M. DiVenere, Y. Liu, A. G. Lee, D. Amengor, K. N. Dalby, S. D′Arcy, J. S. Mclellan, J. A. Maynard, Identification of a conserved neutralizing epitope present on spike proteins from all highly pathogenic coronaviruses. bioRxiv (2021), p. 2021.01.31.428824, , doi: 10.1101/2021.01.31.428824.
28. P. V. Raghuvamsi, N. K. Tulsian, F. Samsudin, X. Qian, K. Purushotorman, G. Yue, M. M. Kozma, W. Y. Hwa, J. Lescar, P. J. Bond, P. A. MacAry, G. S. Anand, SARS-COV-2 S protein: ACE2 interaction reveals novel allosteric targets. Elife. 10 (2021), doi: 10.7554/eLife.63646.
29. R. J. Edwards, K. Mansouri, V. Stalls, K. Manne, B. Watts, R. Parks, K. Janowska, S. M. C. Gobeil, M. Kopp, D. Li, X. Lu, Z. Mu, M. Deyton, T. H. Oguin 3rd, J. Sprenz, W. Williams, K. O. Saunders, D. Montefiori, G. D. Sempowski, R. Henderson, S. Munir Alam, B. F. Haynes, P. Acharya, Cold sensitivity of the SARS-COV-2 spike ectodomain. Nat. Struct. Mol. Biol. 28, 128-131 (2021).
30. C .- L. Hsieh, J. A. Goldsmith, J. M. Schaub, A. M. DiVenere, H .- C. Kuo, K. Javanmardi, K. C. Le, D. Wrapp, A. G. Lee, Y. Liu, C .- W. Chou, P. O. Byrne, C. K. Hjorth, N. V. Johnson, J. Ludes-Meyers, A. W. Nguyen, J. Park, N. Wang, D. Amengor, J. J. Lavinder, G. C. Ippolito, J. A. Maynard, I. J. Finkelstein, J. S. Mclellan, Structure-based design of prefusion-stabilized SARS-COV-2 spikes. Science. 369, 1501-1505 (2020).
31. R. Henderson, R. J. Edwards, K. Mansouri, K. Janowska, V. Stalls, S. M. C. Gobeil, M. Kopp, D. Li, R. Parks, A. L. Hsu, M. J. Borgnia, B. F. Haynes, P. Acharya, Controlling the SARS-COV-2 spike glycoprotein conformation. Nat. Struct. Mol. Biol. 27, 925-933 (2020).
32. R. Yan, Y. Zhang, Y. Li, L. Xia, Y. Guo, Q. Zhou, Structural basis for the recognition of SARS-COV-2 by full-length human ACE2. Science. 367, 1444-1448 (2020).
33. Q. Wang, Y. Zhang, L. Wu, S. Niu, C. Song, Z. Zhang, G. Lu, C. Qiao, Y. Hu, K .- Y. Yuen, Q. Wang, H. Zhou, J. Yan, J. Qi, Structural and Functional Basis of SARS-COV-2 Entry by Using Human ACE2. Cell. 181, 894-904.e9 (2020).
34. T. N. Starr, A. J. Greaney, A. Addetia, W. W. Hannon, M. C. Choudhary, A. S.

Dingens, J. Z. Li, J. D. Bloom, Prospective mapping of viral mutations that escape antibodies used to treat COVID-19. Science. 371, 850-854 (2021).
35. M. S. A. Gilman, P. Furmanova-Hollenstein, G. Pascual, A. B van 't Wout, J. P. M. Langedijk, J. S. Mclellan, Transient opening of trimeric prefusion RSV F proteins. Nat. Commun. 10, 2105 (2019).
36. A. A. Albertini, C. Mérigoux, S. Libersou, K. Madiona, S. Bressanelli, S. Roche, J. Lepault, R. Melki, P. Vachette, Y. Gaudin, Characterization of monomeric intermediates during VSV glycoprotein structural transition. PLOS Pathog. 8, e1002556 (2012).
37. I. S. Kim, S. Jenni, M. L. Stanifer, E. Roth, S. P. J. Whelan, A. M. van Oijen, S. C. Harrison, Mechanism of membrane fusion induced by vesicular stomatitis virus G protein. Proc. Natl. Acad. Sci. U. S. A. 114, E28-E36 (2017).
38. D. M. Eckert, P. S. Kim, Mechanisms of Viral Membrane Fusion and Its Inhibition. Annu. Rev. Biochem. 70, 777-810 (2001).
39. J. R. Byrum, E. Waltari, O. Janson, S .- M. Guo, J. Folkesson, B. B. Chhun, J. Vinden, I. E. Ivanov, M. L. Forst, H. Li, A. G. Larson, W. Wu, C. M. Tato, K. M. Mccutcheon, M. J. Peluso, T. J. Henrich, S. G. Deeks, M. Prakash, B. Greenhouse, J. E. Pak, S. B. Mehta, multiSero: open multiplex-ELISA platform for analyzing antibody responses to SARS-COV-2 infection. medRxiv (2021), doi: 10.1101/2021.5.7.21249238.
40. A. Glasgow, J. Glasgow, D. Limonta, P. Solomon, I. Lui, Y. Zhang, M. A. Nix, N. J. Rettko, S. Zha, R. Yamin, K. Kao, O. S. Rosenberg, J. V. Ravetch, A. P. Wiita, K. K. Leung, S. A. Lim, X. X. Zhou, T. C. Hobman, T. Kortemme, J. A. Wells, Engineered ACE2 receptor traps potently neutralize SARS-COV-2. Proc. Natl. Acad. Sci. U. S. A. (2020), doi: 10.1073/pnas.2016093117.
41. Erlanson D.A. (2011) Introduction to Fragment-Based Drug Discovery. In: Davies T., Hyvönen M. (eds) Fragment-Based Drug Discovery and X-Ray Crystallography. Topics in Current Chemistry, vol 317. Springer, Berlin, Heidelberg
42. Murray, C., Rees, D. The rise of fragment-based drug discovery. Nature Chem. 1:187-192 (2009)
43. J. Lyu et al., Ultra-large library docking for discovering new chemotypes. Nature 566, 224-229 (2019)
44. R. Abagyan and M. Totrov, High-throughput docking for lead generation, Current Opinion in Chemical Biology, Vol. 5, 375-382 (2001).
45. M. L. Lamb et al., Design, docking, and evaluation of multiple libraries against multiple targets, Proteins, Vol. 42, 296-318 (2001).
46. B. Waszkowycz et al. Large-scale virtual screening for discovering leads in the postgenomic era, IBM Systems Journal, Vol. 40, No. 2 (2001).

47. Amanat et al., 2020, “A serological assay to detect SARS-COV-2 seroconversion in humans.” Nature Medicine 26:1033-1036.

1. A method of determining (a) a distribution of a SARS-COV-2 Spike protein an aqueous solution between a first conformation and a second conformation, or (b) if a ligand is capable of stabilizing the first conformation and/or the second conformation of the SARS-COV-2 Spike protein, the method comprising:
providing the aqueous solution of the SARS-COV-2 Spike protein and, optionally, the ligand;
performing hydrogen/deuterium exchange mass spectrometry (HDX-MS) analysis of the aqueous solution of the SARS-COV-2 Spike protein, thereby generating HDX-MS analysis data;
using a computer, calculating deuterium incorporation data from the HDX-MS analysis data; and,
using a computer, determining, from the deuterium incorporation data, the distribution of the SARS-COV-2 Spike protein in the aqueous solution between the first conformation and the second conformation, wherein the SARS-COV-2 Spike protein comprises one or more peptides having first deuterium incorporation data in the first conformation of the SARS-COV-2 Spike protein and second deuterium incorporation data in the second conformation of the SARS-COV-2 Spike protein,
wherein the ligand is capable of stabilizing the first conformation of the SARS-COV-2 Spike protein when a proportion of the SARS-COV-2 Spike protein found in the first conformation is increased in presence of the ligand as compared to absence of the ligand,
and wherein the ligand is capable of stabilizing the second conformation of the SARS-COV-2 Spike protein when a proportion of the SARS-COV-2 Spike protein found in the second conformation is increased in the presence of the ligand as compared to the absence of the ligand.

2. (canceled)

3. The method of claim 1, wherein the ligand is a small molecule, an antibody or a fragment thereof, or a polypeptide.

4. The method of claim 1,
wherein the HDX-MS analysis comprises:
incubating the aqueous solution of the SARS-COV-2 Spike protein with D₂O, thereby generating a sample of partially deuterated SARS-COV-2 Spike protein;
quenching the sample of the partially deuterated SARS-COV-2 Spike protein with a cold acid solution;
subjecting the quenched sample of the partially deuterated SARS-COV-2 Spike protein to protease digestion, thereby generating a mixture of partially deuterated proteolytic peptides of the SARS-COV-2 Spike protein; and,
analyzing the mixture of partially deuterated proteolytic peptides of the SARS-COV-2 Spike protein by mass-spectrometry (MS) to generate the HDX-MS data.

5. The method of claim 1, wherein the deuterium incorporation data comprises a spectrum of mass-to-charge (m/z) ratios detected by the MS for each of the partially deuterated proteolytic peptides of the SARS-COV-2 Spike protein.

6. The method of claim 5, wherein the spectrum of mass-to-charge (m/z) ratios for one or more peptides having the first deuterium incorporation data in the first conformation of the SARS-COV-2 Spike protein and the second deuterium incorporation data in the second conformation of the SARS-COV-2 Spike protein is a bimodal spectrum comprising a sum of the first deuterium incorporation data and the second deuterium incorporation data.

7. The method of claim 6, wherein the determining the distribution of the SARS-COV-2 Spike protein in the aqueous solution between the first conformation and the second conformation comprises:
fitting a sum of two distribution functions to the bimodal spectrum; and,
calculating an area under each of the two distribution functions to determine proportions of the first conformation and the second conformation.

8. The method of claim 7, wherein the two distribution functions are Gaussian.

9. The method of claim 1, wherein the one or more peptides having the first deuterium incorporation data in the first conformation of the SARS-COV-2 Spike protein and the second deuterium incorporation data in the second conformation of the SARS-COV-2 Spike protein comprise an amino acid sequence homologous to amino acid residues 291-300 of SEQ ID NO:1, 291-303 of SEQ ID NO:1, 553-568 of SEQ ID NO:1, 626-636 of SEQ ID NO:1, 662-673 of SEQ ID NO:1, 878-901 of SEQ ID NO:1, 878-902 of SEQ ID NO: 1, 904-916 of SEQ ID NO: 1, 962-967 of SEQ ID NO: 1, C, 982-1001 of SEQ ID NO:1, 978-1001 of SEQ ID NO:1, 982-1001 of SEQ ID NO:1, 1002-1024 of SEQ ID NO:1, 1146-1166 of SEQ ID NO:1, 1179-1186 of SEQ ID NO: 1, or 1187-1197 of SEQ ID NO:1, and/or wherein the second conformation of the SARS-COV-2 Spike protein comprises solvent-exposed amino acid residues in SARS-COV-2 Spike protein acid sequence in regions homologous to one or more of amino acid residues 291-305 of SEQ ID NO:1, 870-916 of SEQ ID NO: 1, 553-574 of SEQ ID NO:1, 626-636 of SEQ ID NO:1, 662-673 of SEQ ID NO: 1, 962-1024 of SEQ ID NO:1, 1146-1166 of SEQ ID NO:1, 1187-1196 of SEQ ID NO: 1, 962-1024 of SEQ ID NO:1, 1146-1166 of SEQ ID NO:1, or 1187-1196 of SEQ ID NO:1.

10-11. (canceled)

12. The method of claim 1, wherein the second conformation of the SARS-COV-2 Spike protein comprises a binding site for 3A3 antibody in a region homologous to amino acid residues 978-1001 of SEQ ID NO:1.

13. The method of claim 12, wherein the binding site for 3A3 antibody is occluded from solvent in a complex of the second conformation of the SARS-COV-2 Spike protein and 3A3 antibody.

14. A method of (a) detecting binding of a ligand to a second conformation of a SARS-COV-2 Spike protein or (b) identifying a ligand capable of binding to the second conformation of the SARS-COV-2 Spike protein, the method comprising:
providing an aqueous solution comprising the SARS-COV-2 Spike in the second conformation;
contacting the ligand with the aqueous solution comprising the SARS-COV-2 Spike protein in the second conformation; and
in (a), after the contacting, performing an in vitro analytical method to detect binding of the ligand to SARS-COV-2 Spike protein, wherein the SARS-COV-2 Spike protein comprises one or more peptides having second deuterium incorporation data obtained by hydrogen/deuterium exchange mass spectrometry (HDX-MS) analysis in the second conformation of the SARS-COV-2 Spike protein, and first deuterium incorporation data obtained by HDX-MS analysis in a first conformation of the SARS-COV-2 Spike protein, wherein the first deuterium incorporation data is different from the second deuterium incorporation data
or, in (b), after the contacting, performing HDX-MS analysis to detect if one or more peptides of the SARS-COV-2 Spike protein that are more solvent exposed in the second conformation of the SARS-COV-2 Spike protein than in a first conformation of the SARS-COV-2 Spike protein become less solvent after the contacting with the ligand, thereby identifying the ligand as capable of binding to the second conformation of the SARS-COV-2 Spike protein.

15. The method of claim 14, wherein the ligand is a small molecule, an antibody or a fragment thereof, or a polypeptide.

16. The method of claim 14, wherein the in vitro analytical method comprises one or more bio-layer interferometry, surface-plasmon resonance, gel-shift assay, fluorescence polarization assay, fluorescence anisotropy assay, or isothermal calorimetry.

17. The method of claim 14 wherein, in (a), the one or more peptides having the first deuterium incorporation data in the first conformation of the SARS-COV-2 Spike protein and the second deuterium incorporation data in the second conformation of the SARS-COV-2 Spike protein, or, in (b), the one or more peptides comprise an amino acid sequence homologous to amino acid residues 291-300 of SEQ ID NO:1, 291-303 of SEQ ID NO:1, 553-568 of SEQ ID NO:1, 626-636 of SEQ ID NO:1, 662-673 of SEQ ID NO:1, 878-901 of SEQ ID NO:1, 878-902 of SEQ ID NO:1, 904-916 of SEQ ID NO:1, 962-967 of SEQ ID NO:1, C, 982-1001 of SEQ ID NO:1, 978-1001 of SEQ ID NO:1, 982-1001 of SEQ ID NO:1, 1002-1024 of SEQ ID NO: 1, 1146-1166 of SEQ ID NO: 1, 1179-1186 of SEQ ID NO:1, or 1187-1197 of SEQ ID NO:1.

18. The method of claim 14, wherein the second conformation of the SARS-COV-2 Spike protein comprises solvent-exposed amino acid residues in SARS-COV-2 Spike protein acid sequence in regions homologous to one or more of amino acid residues 291-305 of SEQ ID NO:1, 870-916 of SEQ ID NO:1, 553-574 of SEQ ID NO: 1, 626-636 of SEQ ID NO:1, 662-673 of SEQ ID NO: 1, 962-1024 of SEQ ID NO:1, 1146-1166 of SEQ ID NO:1, 1187-1196 of SEQ ID NO:1, 962-1024 of SEQ ID NO:1, 1146-1166 of SEQ ID NO:1, or 1187-1196 of SEQ ID NO:1.

19-22. (canceled)

23. A method of identifying a ligand capable of binding to SARS-COV-2 Spike protein, comprising:
I) screening in silico a ligand library for candidate ligands capable of binding to a first conformation of the SARS-COV-2 Spike protein, a second conformation of the SARS-COV-2 Spike protein, or both to the first and the second conformation of the SARS-COV-2 Spike protein wherein three-dimensional models of the first conformation and the second conformation of the SARS-COV-2 Spike protein are computationally derived and incorporate solvent accessibility information based on deuterium incorporation data obtained by hydrogen/deuterium exchange mass spectrometry (HDX-MS) analysis; and,
II) evaluating the candidate ligands identified in step (I) through one or more in vitro analytical method for their ability to bind to the SARS-COV-2 Spike protein.

24. The method of claim 23, wherein in step (I) the ligand library is screened using computational docking for the candidate ligands, and wherein the screening of the ligand library to identify a candidate ligand includes:
determining a docking score between the candidate ligand and the first conformation of the SARS-COV-2 Spike protein, the second conformation of the SARS-COV-2 Spike protein, or both the first and the second conformation of the SARS-COV-2 Spike protein;
comparing the docking score to a threshold for binding to the SARS-COV-2 Spike protein; and, identifying the candidate ligand based on the docking score being above the threshold. 25 (Original) A method of identifying a ligand capable of binding to a first conformation and/or a second conformation of a SARS-COV-2 Spike protein, the method comprising:
I) identifying in silico a test ligand capable of interacting with the first conformation of the SARS-COV-2 Spike protein, the second conformation of the SARS-COV-2 Spike protein, or both to the first and the second conformation of the SARS-COV-2 Spike protein wherein three-dimensional models of the first conformation and the second conformation of the SARS-COV-2 Spike protein are computationally derived and incorporate solvent accessibility information based on deuterium incorporation obtained by hydrogen/deuterium exchange mass spectrometry (HDX-MS) analysis; and,
II) evaluating the test ligand identified in step (I) through one or more in vitro analytical method for its ability to bind to the SARS-COV-2 Spike protein.

26. The method of claim 25, wherein in step (I) the test ligand is identified using computational docking, comprising:
determining a docking score between the test ligand and the first conformation of the SARS-COV-2 Spike protein, the second conformation of the SARS-COV-2 Spike protein, or both the first and the second conformation of the SARS-COV-2 Spike protein;
comparing the docking score to a threshold for binding to the SARS-COV-2 Spike protein; and,
identifying the test ligand based on the docking score being above the threshold. 27 (Currently Amended) The method of claim 23, wherein the solvent accessibility information comprises hydrogen exchange rates calculated based on the deuterium incorporation data obtained by the HDX-MS analysis.

28-39. (canceled)
